(19) United States
(12) Patent Application Publication
(10) Pub. No.: US 2017/0088545 A1
(43) Pub. Date: Mar. 30, 2017

(54) CARBOXAMIDE INHIBITORS

(71) Applicant: Norvartis AG, Basel (CH)

(72) Inventors: Luca ARISTA, Riehen (CH); Simona COTESTA, Basel (CH); Pier D'ALESSANDRO, Basel (CH); Anne-Marie EDWARDS, Godalming (GB); Rene LATTMANN, Oberwil (CH); Dimitrios LIZOS, Basel (CH); Robert Alexander PULZ, Basel (CH); Lisa Ann ROONEY, Barnstead (GB); Nichola SMITH, Cambridge, MA (US); Jessica Louise TAYLOR, Dorking (GB); Thomas J. TROXLER, Laufen (CH)

(73) Assignee: Novartis AG, Basel (CH)

(21) Appl. No.: 15/310,948

(22) PCT Filed: May 14, 2015
(86) PCT No.: PCT/IB2015/001498

§ 371 Date: Nov. 14, 2016

Related U.S. Application Data
(60) Provisional application No. 61/993,046, filed on May 14, 2014.

(51) Int. Cl. C07D 413/12; C07D 413/14; A61K 31/422

(52) U.S. Cl. CPC C07D 413/12; A61K 31/422; C07D 413/14

(57) ABSTRACT

The present invention provides a compound of formula I

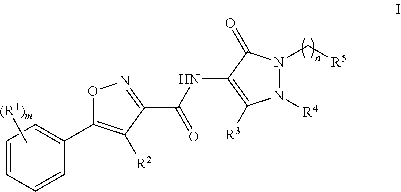

The compounds of formula I demonstrate properties as Smurf-1 inhibitors and are thus useful in the treatment of a range of disorders, particularly pulmonary arterial hypertension.

The present invention describes organic compounds and/or salts thereof that are useful in therapy. The compounds demonstrate properties as selective Smurf-1 inhibitors and may thus be useful in the treatment of a range of disorders, such as for example, pulmonary arterial hypertension, glaucoma, hereditary hemorrhagic telangiectasia (HHT), proteinuria, wound healing, as well as COPD and asthma.

Smurf-1 (Smad ubiquitination regulatory factor 1) is a member of the HECT family of E3 ubiquitin ligase marking specific substrates for proteolytic degradation via the ubiquitin-dependent proteolytic pathway. Major substrates of Smurf-1 include RhoA, bone morphogenetic protein (BMP) receptor (BMPR) 1 and 2, smad1 and 5, TNFα receptor associated factor (TRAF) 6 and myD88 (Andrews, P. S. et al. Assay Drug Dev. Technol. 2010). Given the list of substrates, Smurf-1 has established roles in regulating BMP signaling (Chen, D et al. Growth Factors, 2004), neuronal cell polarity (Stiess, M. and Bradke, F. Neuron, 2011), cell migration (Huang, C. Cell Adh. Migr. 2010), tumor cell invasion (Sahai, E. et al. JCB, 2007), mitochondrial autophagy (Orvedahl, A. Nature, 2011) mesenchymal stem cell proliferation (Zhao, L. et al. J. Bone Miner. Res. 2010) and epithelial-mesenchymal transition (EMT) (Ozdamar, B et al. Science 2005).

Pulmonary arterial hypertension (PAH) is a life-threatening aggressive and complex disease of multiple etiologies, characterized by a progressive pulmonary vasculopathy leading to right ventricular hypertrophy/failure and in most cases premature death. Current pharmacological therapies are palliative. Whilst improvements in life expectancy have been observed, current therapies, which focus on altering the vasoconstrictive elements of the disease, do not halt or reverse progression of the disease, and transplantation (double lung or heart-lung) remains the only curative treatment. Given the limited effect of current treatment classes, novel therapies targeting the underlying progressive pulmonary vascular remodeling of PAH are needed.

Germline mutations in the transforming growth factor β (TGF-β) superfamily receptor bone morphogenetic protein receptor II (BMPR-II) gene are prevalent in seventy percent of heritable and some sporadic forms of idiopathic PAH (IPAH). Bone morphogenetic proteins are signaling molecules that belong to the TGF-β superfamily. Bone morphogenetic proteins were originally identified by their ability to induce formation of cartilage and bone, and subsequently identified to be multifunctional proteins that regulate a wide spectrum of function such as proliferation, differentiation, and apoptosis in a large variety of cell types, including osteoblasts, epithelial cells, neurons, immune cells, and smooth muscle cells. So far, >20 mammalian BMPs have been identified, but only three type I and three type II receptors (BMPR-I and BMPR-II, respectively) that are capable of binding with BMPs have been cloned in mammals. Bone morphogenetic proteins are synthesized and secreted from a variety of cell types, including pulmonary vascular smooth muscle cells and endothelial cells. In addition to mutations in BMPR-I and -II, lungs from patients with non-familial PAH display markedly reduced levels of vascular BMPR-1 and -II implying a central role for disrupted BMP signaling in many forms of PAH (Du, L et al. N. Eng. J. Med, 2003). Restoration of BMP signaling in the pulmonary vasculature of PAH patients is therefore of considerable interest in the development of novel anti-remodeling therapeutics for the treatment of PAH.

Smurf-1 has been shown to mediate degradation of BMPR-I, -II and smad1 and 5 in a variety of cell types including osteoblasts (Zhao, M et al. JBC, 2003), myoblasts (Ying, S X et al. JBC, 2003), lung epithelium (Shi W, et al. Am. J. Physiol. Cell. Mol. Physiol, 2004), neuronal tissue (Kallan, T et al. Mol. Cell. Biol, 2009) and endocardial cells (Towsend, T A, et al. Cells Tissues Organs, 2011). Recently, the first evidence has emerged supporting a role for Smurf-1 in PAH where enhanced levels of Smurf-1 were observed in the chronic hypoxia and monocrotaline pre-clinical in-vivo models of PAH and associated with down-regulation of BMPR1 and 2 (Murakami, K, et al. Exp. Biol. Med, 2010 and Yang, J. et al. Circ. Res, 2010).

The following compounds are known:
N-(2,3-dihydro-1,5-dimethyl-3-oxo-2-phenyl-1H-pyrazol-4-yl)-5-(2-fluorophenyl)isoxazole-3-carboxamide (registry number 1302184-45-7; Library: FCH Group);
N-(2,3-dihydro-1,5-dimethyl-3-oxo-2-phenyl-2,3-dihydro-1H-pyrazol-4-yl)-5-(4-fluorophenyl)isoxazole-3-carboxamide (registry number 688050-41-1; Library: ChemDiv. Inc);
N-(2,3-dihydro-1,5-dimethyl-3-oxo-2-phenyl-2,3-dihydro-1H-pyrazol-4-yl)-5-(4-ethoxyphenyl)isoxazole-3-carboxamide (registry number 898468-25-2; Library: Princeton Biomolec. Res.);
N-(2,3-dihydro-1,5-dimethyl-3-oxo-2-phenyl-2,3-dihydro-1H-pyrazol-4-yl)-5-(3-chlorophenyl)isoxazole-3-carboxamide (registry number 912760-79-3; Library: ChemDiv. Inc.);
N-(2,3-dihydro-1,5-dimethyl-3-oxo-2-phenyl-2,3-dihydro-1H-pyrazol-4-yl)-5-(4-bromophenyl)isoxazole-3-carboxamide (registry number 912791-65-2; Library: Aurora Fine Chemicals);
N-(2,3-dihydro-1,5-dimethyl-3-oxo-2-phenyl-2,3-dihydro-1H-pyrazol-4-yl)-5-(3,4-dimethoxyphenyl)isoxazole-3-carboxamide (registry number 912797-84-3; Library: Aurora Fine Chemicals);
N-(2,3-dihydro-1,5-dimethyl-3-oxo-2-phenyl-2,3-dihydro-1H-pyrazol-4-yl)-5-(3,4-dimethylphenyl)isoxazole-3-carboxamide (registry number 898498-23-2; Library: Princeton Biomolec. Res.);
N-(2,3-dihydro-1,5-dimethyl-3-oxo-2-phenyl-2,3-dihydro-1H-pyrazol-4-yl)-5-(4-methylphenyl)isoxazole-3-carboxamide (registry number 717878-93-8; Library: ChemBridge);
N-(2,3-dihydro-1,5-dimethyl-3-oxo-2-phenyl-2,3-dihydro-1H-pyrazol-4-yl)-5-(3,4-dichlorophenyl)isoxazole-3-carboxamide (registry number 912775-67-8; Library: Aurora Fine Chemicals);
N-(2,3-dihydro-1,5-dimethyl-3-oxo-2-phenyl-2,3-dihydro-1H-pyrazol-4-yl)-5-(4-chlorophenyl)isoxazole-3-carboxamide (registry number 912787-86-1; Library: Aurora Fine Chemicals);
N-(2,3-dihydro-1,5-dimethyl-3-oxo-2-phenyl-2,3-dihydro-1H-pyrazol-4-yl)-5-(4-methoxyphenyl)isoxazole-3-carboxamide (registry number 912795-89-2; Library: Aurora Fine Chemicals); and
N-(2,3-dihydro-1,5-dimethyl-3-oxo-2-phenyl-2,3-dihydro-1H-pyrazol-4-yl)-5-(3,4-difluorophenyl)isoxazole-3-carboxamide (registry number 1206037-29-7; Library: Princeton Biomolec. Res.).

US Patent Application Number US 20090163545 A1 discloses the following compounds: N-(2,3-dihydro-1,5-dimethyl-3-oxo-2-phenyl-1H-pyrazol-4-yl)-5-phenyl-isoxazole-3-carboxamide (Registry Number 712347-96-1), N-(2,3-dihydro-1,5-dimethyl-3-oxo-2-phenyl-1H-pyrazol-4-yl)-5-(2,5-dimethoxyphenyl)-isoxazole-3-carboxamide (Registry Number 907986-88-3), N-(2,3-dihydro-1,5-dimethyl-3-oxo-2-phenyl-1H-pyrazol-4-yl)-5-(3-methoxyphenyl)-isoxazole-3-carboxamide (Registry Number 912782-55-9) and N-(2,3-dihydro-1,5-dimethyl-3-oxo-2-phenyl-1H-pyrazol-4-yl)-5-(2-methoxyphenyl)-isoxazole-3-carboxamide (Registry Number 907986-85-0),

Hence, it is the object of the invention to provide novel Smurf-1 inhibitors.

The invention pertains to the compounds, methods for using them, and uses thereof as described herein. Examples of compounds of the invention include the compounds according to Formula I or a pharmaceutically acceptable salt or co-crystal thereof, and the compounds of the examples.

The invention, therefore, provides as Embodiment 1 a compound of formula I,

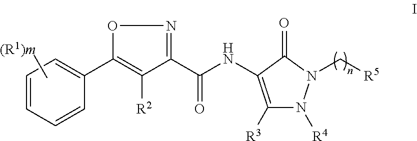

or a pharmaceutically acceptable salt or co-crystal thereof,

wherein:
each R¹ independently represents H, halo, C₁-C₆ alkyl, C₁-C₆ haloalkyl, C₁-C₆ alkoxy, (C₁-C₆ alkoxy) C₁-C₆ alkyl-, C₁-C₆ alkylthio, C₁-C₆ haloalkoxy, di(C₁-C₆ alkyl)amino-, C₁-C₆ alkylamino-, amino, C₃-C₇ cycloalkyl, (C₃-C₇ Cycloalkyl)C₁-C₄ alkyl- or (C₃-C₇ cycloalkyl)C₁-C₄ alkoxy; or two adjacent R¹ substituents may be taken together with the carbon atoms to which they are attached to form C₃-C₇ cycloalkyl, Het¹, or Het²;
R² represents H, halo, cyano, C₁-C₆ alkyl, C₁-C₆ haloalkyl, C₁-C₆ alkoxy, (C₁-C₆ alkoxy)C₁-C₆ alkyl-, C₁-C₆ haloalkoxy, C₃-C₇ cycloalkyl, (C₃-C₇ cycloalkyl)C₁-C₆ alkyl- or (C₃-C₇ cycloalkyl)C₁-C₆ alkoxy-;
R³ represents H, C₁-C₆ alkyl, C₁-C₆ haloalkyl, C₁-C₆ alkoxy, C₁-C₆ haloalkoxy, (C₁-C₆ alkoxy)C₁-C₆ alkyl-, (C₁-C₆ haloalkoxy)C₁-C₆ alkyl-;
R⁴ represents C₁-C₆ alkyl, which C₁-C₆ alkyl is unsubstituted or substituted by one or two halo, C₁-C₆ alkoxy or C₁-C₆ haloalkoxy groups;
R⁵ represents C₃-C₇ cycloalkyl, or phenyl, which C₃-C₇ cycloalkyl or phenyl is unsubstituted or substituted by one or two halo, C₁-C₆ alkyl, C₁-C₆ haloalkyl, C₁-C₆ alkoxy or C₁-C₆ haloalkoxy groups;
n represents an integer selected from 0 and 1;
m represents an integer selected from 0, 1 and 2;
Het¹ represents a 3 to 7 membered heterocyclic ring containing 1 or 2 heteroatoms selected from N, O and S; and
Het² represents a 5 or 6 membered heteroaryl ring containing a) 0 to 2 nitrogen atoms and 1 oxygen or sulphur atom, or b) 1 to 4 nitrogen atoms;
with the proviso that the compound of formula (I) is not one of the following compounds:
N-(2,3-dihydro-1,5-dimethyl-3-oxo-2-phenyl-2,3-dihydro-1H-pyrazol-4-yl)-5-(2-fluorophenyl)isoxazole-3-carboxamide;
N-(2,3-dihydro-1,5-dimethyl-3-oxo-2-phenyl-2,3-dihydro-1H-pyrazol-4-yl)-5-(4-fluorophenyl)isoxazole-3-carboxamide;
N-(2,3-dihydro-1,5-dimethyl-3-oxo-2-phenyl-2,3-dihydro-1H-pyrazol-4-yl)-5-(4-ethoxyphenyl)isoxazole-3-carboxamide;
N-(2,3-dihydro-1,5-dimethyl-3-oxo-2-phenyl-2,3-dihydro-1H-pyrazol-4-yl)-5-(3-chlorophenyl)isoxazole-3-carboxamide;
N-(2,3-dihydro-1,5-dimethyl-3-oxo-2-phenyl-2,3-dihydro-1H-pyrazol-4-yl)-5-(4-bromophenyl)isoxazole-3-carboxamide;
N-(2,3-dihydro-1,5-dimethyl-3-oxo-2-phenyl-2,3-dihydro-1H-pyrazol-4-yl)-5-(3,4-dimethoxyphenyl)isoxazole-3-carboxamide;
N-(2,3-dihydro-1,5-dimethyl-3-oxo-2-phenyl-2,3-dihydro-1H-pyrazol-4-yl)-5-(3,4-dimethylphenyl)isoxazole-3-carboxamide;
N-(2,3-dihydro-1,5-dimethyl-3-oxo-2-phenyl-2,3-dihydro-1H-pyrazol-4-yl)-5-(4-methylphenyl)isoxazole-3-carboxamide;
N-(2,3-dihydro-1,5-dimethyl-3-oxo-2-phenyl-2,3-dihydro-1H-pyrazol-4-yl)-5-(3,4-dichlorophenyl)isoxazole-3-carboxamide;
N-(2,3-dihydro-1,5-dimethyl-3-oxo-2-phenyl-2,3-dihydro-1H-pyrazol-4-yl)-5-(4-chlorophenyl)isoxazole-3-carboxamide;
N-(2,3-dihydro-1,5-dimethyl-3-oxo-2-phenyl-2,3-dihydro-1H-pyrazol-4-yl)-5-(4-methoxyphenyl)isoxazole-3-carboxamide;
N-(2,3-dihydro-1,5-dimethyl-3-oxo-2-phenyl-2,3-dihydro-1H-pyrazol-4-yl)-5-(3,4-difluorophenyl)isoxazole-3-carboxamide;
N-(2,3-dihydro-1,5-dimethyl-3-oxo-2-phenyl-1H-pyrazol-4-yl)-5-phenyl-isoxazole-3-carboxamide;
N-(2,3-dihydro-1,5-dimethyl-3-oxo-2-phenyl-1H-pyrazol-4-yl)-5-(2,5-dimethoxyphenyl)-isoxazole-3-carboxamide;
N-(2,3-dihydro-1,5-dimethyl-3-oxo-2-phenyl-1H-pyrazol-4-yl)-5-(3-methoxyphenyl)-isoxazole-3-carboxamide; and
N-(2,3-dihydro-1,5-dimethyl-3-oxo-2-phenyl-1H-pyrazol-4-yl)-5-(2-methoxyphenyl)-isoxazole-3-carboxamide.

In another embodiment, the invention pertains to compounds of Formula I and/or pharmaceutically acceptable salts thereof.

### DEFINITIONS

Terms used in the specification have the following meanings:
“Smurf-1”, as used herein, also refers to Smurf1 and SMURF1.
“Substituted” means the group referred to can be substituted at one or more positions by any one or any combination of the radicals listed thereafter.
“Halo” or “Halogen”, as used herein, may be fluorine, chlorine, bromine or iodine.
“Alkyl”, as used herein, denotes straight chain or branched alkyl having the enumerated carbon atoms, e.g. methyl, ethyl, propyl, isopropyl, butyl, isobutyl, sec-butyl and tert-butyl.
“Alkoxy”, as used herein, denotes straight chain or branched alkoxy having the enumerated carbon atoms, e.g. methoxy, ethoxy, propoxy, isopropoxy, butoxy, isobutoxy, sec-butoxy and tert-butoxy.
“Haloalkyl” or “Haloalkoxy”, as used herein, denotes straight chain or branched alkyl or alkoxy having the enumerated carbon atoms with at least one hydrogen replaced by a halogen, e.g. where the halogen is fluorine, CF₃CF₂—, (CF₃)₂CH—, CH₃—CF₂—, CF₃CF₂—, CF₃, CF₂H—, CF₃CF₂CHCF₃ or CF₃CF₂CF₂CF₂—.
“C₃-C₇Cycloalkyl”, as used herein, denotes a fully saturated carbocyclic ring selected from cyclopropyl, cyclobutyl, cyclopentyl, cyclohexyl and cycloheptyl.

Various embodiments of the invention are described herein. It is understood that any and all embodiments of the present invention may be taken in conjunction with any other embodiment to describe additional embodiments of the present invention. Furthermore, any elements of an embodiment are meant to be combined with any and all other elements from any of the embodiments to describe additional embodiments. It is understood by those skilled in the art that combinations of substituents where not possible are not an aspect of the present invention.

### Embodiment 2

A compound or pharmaceutically acceptable salt thereof according to Embodiment 1, wherein each R¹ independently represents halo, C₁-C₄ alkyl, C₁-C₄ haloalkyl, C₁-C₄ alkoxy, (C₁-C₄ alkoxy)C₁-C₄ alkyl-C₁-C₄ alkylthio, C₁-C₄ haloalkoxy, di(C₁-C₄ alkyl)amino-, C₁-C₄ alkylamino-, amino, C₃-C₇ cycloalkyl, (C₃-C₇ cycloalkyl)C₁-C₄ alkyl- or (C₃-C₇ cycloalkyl)C₁-C₄ alkoxy-; or

two adjacent R¹ substituents may be taken together with the carbon atoms to which they are attached to form Het¹, or Het²;

### Embodiment 3

A compound or pharmaceutically acceptable salt thereof according to Embodiment 1 or 2, wherein each R¹ independently represents Br, Cl, F, methyl, ethyl, trifluoromethyl, methoxy, ethoxy, methoxymethyl, methylthio, difluoromethoxy, trifluoromethoxy, di(methyl)amino, methylamino, amino, cyclopropyl, (cyclopropyl)C₁-C₂ alkyl- or (cyclopropyl)C₁-C₂ alkoxy-; or

two adjacent R¹ substituents may be taken together with the carbon atoms to which they are attached to form a furan, tetrahydrofuran, pyran or tetrahydropyran ring.

### Embodiment 4

A compound or pharmaceutically acceptable salt thereof according to any preceding Embodiment, wherein, R² represents halo (e.g. Br, Cl, F); cyano, C₁-C₆ alkyl (e.g. methyl, ethyl, n-propyl or isobutyl); C₁-C₆ haloalkyl (e.g. trifluoromethyl); C₁-C₆ alkoxy (e.g. methoxy or isopropoxy); C₁-C₆ haloalkoxy (e.g. trifluoromethoxy); or C₃-C cycloalkyl (e.g cyclopropyl).

### Embodiment 5

A compound or pharmaceutically acceptable salt thereof according to any preceding Embodiment, wherein, R² represents Br, Cl, F cyano, methyl, ethyl, trifluoromethyl, methoxy, trifluoromethoxy or cyclopropyl.

### Embodiment 6

A compound or pharmaceutically acceptable salt thereof according to any preceding Embodiment, wherein R³ represents C₁-C₆ alkyl.

### Embodiment 7

A compound or pharmaceutically acceptable salt thereof according to any preceding Embodiment, wherein R³ represents methyl or ethyl.

### Embodiment 8

A compound or pharmaceutically acceptable salt thereof according to any preceding Embodiment, wherein R⁴ represents C₁-C₆ alkyl.

### Embodiment 9

A compound or pharmaceutically acceptable salt thereof according to any preceding Embodiment, wherein R⁴ represents methyl or ethyl.

### Embodiment 10

A compound or pharmaceutically acceptable salt thereof according to any preceding Embodiment, wherein R³ represents methyl and R⁴ represents methyl.

### Embodiment 11

A compound or pharmaceutically acceptable salt thereof according to any preceding Embodiment, wherein R⁵ represents C₃-C₆ cycloalkyl or phenyl, which C₃-C₆ cycloalkyl or phenyl is unsubstituted or substituted by one or two halo, C₁-C₆ alkyl, C₁-C₆ haloalkyl, C₁-C₆ alkoxy or C₁-C₆ haloalkoxy groups.

### Embodiment 12

A compound or pharmaceutically acceptable salt thereof according to any preceding Embodiment, wherein R⁵ represents cyclohexyl, or phenyl, which cyclohexyl or phenyl is unsubstituted or substituted by one or two halo, C₁-C₆ alkyl, C₁-C₆ haloalkyl, C₁-C₆ alkoxy or C₁-C₆ haloalkoxy groups.

### Embodiment 13

A compound or pharmaceutically acceptable salt thereof according to any preceding Embodiment, wherein R⁵ represents cyclohexyl, or phenyl, which cyclohexyl or phenyl is unsubstituted or substituted by one or two halo, C₁-C₆ alkyl or C₁-C₆ haloalkyl groups.

### Embodiment 14

A compound or pharmaceutically acceptable salt thereof according to any preceding Embodiment, wherein when R⁵ is phenyl, the phenyl is substituted by one or two groups as defined above.

### Embodiment 15

A compound or pharmaceutically acceptable salt thereof according to any preceding Embodiment, wherein m represents 1 or 2.

### Embodiment 16

A compound or pharmaceutically acceptable salt thereof according to any preceding Embodiment, wherein when m is 1, the R¹ substituent is positioned in the 2- or 4-position of the phenyl ring; and when m is 2, the R¹ substituents are positioned in the 2- and 4-positions of the phenyl ring.

### Embodiment 17

A compound or pharmaceutically acceptable salt thereof according to any of the preceding Embodiments, wherein n represents 0.

### Embodiment 18

A compound of formula I according to any preceding Embodiment, wherein the compound is selected from:
5-(2,4-Dichlorophenyl)-N-(2-(2-fluorophenyl)-1,5-dimethyl-3-oxo-2,3-dihydro-1H-pyrazol-4-yl)-4-methylisoxazole-3-carboxamide;
5-(2-Chloro-4-cyclopropylphenyl)-N-(2-(2-fluorophenyl)-1,5-dimethyl-3-oxo-2,3-dihydro-1H-pyrazol-4-yl)-4-methylisoxazole-3-carboxamide;
4-Chloro-5-(2-chloro-4-(difluoromethoxy)phenyl)-N-(2-(2-fluorophenyl)-1,5-dimethyl-3-oxo-2,3-dihydro-1H-pyrazol-4-yl)isoxazole-3-carboxamide;
N-(2-(2-Fluorophenyl)-1,5-dimethyl-3-oxo-2,3-dihydro-1H-pyrazol-4-yl)-5-(4-methoxyphenyl)-4-methylisoxazole-3-carboxamide;
5-(2-Chloro-4-cyclopropylphenyl)-N-(2-(2-fluorophenyl)-1 trideuteromethyl-5-methyl-3-oxo-2,3-dihydro-1H-pyrazol-4-yl)-4-methylisoxazole-3-carboxamide;
5-(2-Fluoro-4-methoxyphenyl)-N-(2-(2-fluorophenyl)-1,5-dimethyl-3-oxo-2,3-dihydro-1H-pyrazol-4-yl)-4-methylisoxazole-3-carboxamide;
5-(2-Chloro-4-methoxyphenyl)-N-(2-(2-fluorophenyl)-1,5-dimethyl-3-oxo-2,3-dihydro-1H-pyrazol-4-yl)-4-methylisoxazole-3-carboxamide;
N-(2-(2-Fluorophenyl)-1,5-dimethyl-3-oxo-2,3-dihydro-1H-pyrazol-4-yl)-4-methyl-5-(4-(trifluoromethoxy)phenyl)isoxazole-3-carboxamide;
N-(2-(2-Fluorophenyl)-1,5-dimethyl-3-oxo-2,3-dihydro-1H-pyrazol-4-yl)-4-methyl-5-(4-(trifluoromethyl)phenyl)isoxazole-3-carboxamide;
5-(2-Chloro-4-(trifluoromethoxy)phenyl)-N-(2-(2-fluorophenyl)-1,5-dimethyl-3-oxo-2,3-dihydro-1H-pyrazol-4-yl)-4-methylisoxazole-3-carboxamide;
5-(2-Chloro-4-(trifluoromethoxy)phenyl)-N-(2-cyclohexyl-1,5-dimethyl-3-oxo-2,3-dihydro-1H-pyrazol-4-yl)-4-methylisoxazole-3-carboxamide;
N-(2-(2-Fluorophenyl)-1,5-dimethyl-3-oxo-2,3-dihydro-1H-pyrazol-4-yl)-4-methyl-5-(4-(methylthio)phenyl)isoxazole-3-carboxamide;
5-(4-(Dimethylamino)phenyl)-N-(2-(2-fluorophenyl)-1,5-dimethyl-3-oxo-2,3-dihydro-1H-pyrazol-4-yl)-4-methylisoxazole-3-carboxamide;
N-(2-(2-Fluorophenyl)-1,5-dimethyl-3-oxo-2,3-dihydro-1H-pyrazol-4-yl)-5-(4-methoxy-2-(trifluoro methyl)phenyl)-4-methylisoxazole-3-carboxamide;
N-(2-Cyclohexyl-1,5-dimethyl-3-oxo-2,3-dihydro-1H-pyrazol-4-yl)-5-(2,4-dichloro phenyl)-4-methylisoxazole-3-carboxamide;
5-(2-Chloro-4-methoxyphenyl)-N-(2-cyclohexyl-1,5-dimethyl-3-oxo-2,3-dihydro-1H-pyrazol-4-yl)-4-methylisoxazole-3-carboxamide;
N-(2-Cyclohexyl-1,5-dimethyl-3-oxo-2,3-dihydro-1H-pyrazol-4-yl)-5-(4-methoxy-2-(trifluoromethyl)phenyl)-4-methylisoxazole-3-carboxamide;
N-(2-Cyclohexyl-1,5-dimethyl-3-oxo-2,3-dihydro-1H-pyrazol-4-yl)-5-(4-cyclopropyl-2-fluorophenyl)-4-methylisoxazole-3-carboxamide;
5-(4-Cyclopropyl-2-fluorophenyl)-N-(2-(2-fluorophenyl)-1,5-dimethyl-3-oxo-2,3-dihydro-1H-pyrazol-4-yl)-4-methylisoxazole-3-carboxamide;
5-(2-Chloro-4-cyclopropylphenyl)-N-(2-cyclohexyl-1,5-dimethyl-3-oxo-2,3-dihydro-1H-pyrazol-4-yl)-4-methylisoxazole-3-carboxamide;
4-Chloro-5-(2,4-dichlorophenyl)-N-(2-(2-fluorophenyl)-1,5-dimethyl-3-oxo-2,3-dihydro-1H-pyrazol-4-yl)isoxazole-3-carboxamide;
4-Chloro-5-(3-chloro-2-fluoro-4-methoxy phenyl)-N-(2-(2-fluorophenyl)-1,5-dimethyl-3-oxo-2,3-dihydro-1H-pyrazol-4-yl)isoxazole-3-carboxamide;
4-Chloro-5-(2-fluoro-4-methoxyphenyl)-N-(2-(2-fluorophenyl)-1,5-dimethyl-3-oxo-2,3-dihydro-1H-pyrazol-4-yl)isoxazole-3-carboxamide;
4-Chloro-N-(2-cyclohexyl-1,5-dimethyl-3-oxo-2,3-dihydro-1H-pyrazol-4-yl)-5-(2,4-dichlorophenyl)isoxazole-3-carboxamide;
4-Chloro-5-(2-chloro-4-(trifluoromethoxy)phenyl)-N-(2-cyclohexyl-1,5-dimethyl-3-oxo-2,3-dihydro-1H-pyrazol-4-yl)isoxazole-3-carboxamide;
4-Chloro-5-(2-chloro-4-methoxyphenyl)-N-(2-(2-fluorophenyl)-1,5-dimethyl-3-oxo-2,3-dihydro-1H-pyrazol-4-yl)isoxazole-3-carboxamide;
4-Chloro-5-(2-chloro-4-methoxyphenyl)-N-(2-cyclohexyl-1,5-dimethyl-3-oxo-2,3-dihydro-1H-pyrazol-4-yl)isoxazole-3-carboxamide;
4-Chloro-5-(2-chloro-4-(difluoromethoxy)phenyl)-N-(2-cyclohexyl-1,5-dimethyl-3-oxo-2,3-dihydro-1H-pyrazol-4-yl)isoxazole-3-carboxamide;
4-Chloro-5-(2-chloro-4-cyclopropylphenyl)-N-(2-(2-fluorophenyl)-1,5-dimethyl-3-oxo-2,3-dihydro-1H-pyrazol-4-yl)isoxazole-3-carboxamide;
4-Chloro-5-(2-chloro-4-cyclopropylphenyl)-N-(2-cyclohexyl-1,5-dimethyl-3-oxo-2,3-dihydro-1H-pyrazol-4-yl)isoxazole-3-carboxamide;
5-(2-Chloro-4-(trifluoromethoxy)phenyl)-N-(2-cyclohexyl-1,5-dimethyl-3-oxo-2,3-dihydro-1H-pyrazol-4-yl)isoxazole-3-carboxamide;
5-(2-Chloro-4-methoxyphenyl)-4-fluoro-N-(2-(2-fluorophenyl)-1,5-dimethyl-3-oxo-2,3-dihydro-1H-pyrazol-4-yl)isoxazole-3-carboxamide;
4-Bromo-5-(2-chloro-4-methoxyphenyl)-N-(2-cyclohexyl-1,5-dimethyl-3-oxo-2,3-dihydro-1H-pyrazol-4-yl)isoxazole-3-carboxamide;
4-Bromo-5-(2-chloro-4-methoxyphenyl)-N-(2-(2-fluorophenyl)-1,5-dimethyl-3-oxo-2,3-dihydro-1H-pyrazol-4-yl)isoxazole-3-carboxamide;
4-Bromo-N-(2-cyclohexyl-1,5-dimethyl-3-oxo-2,3-dihydro-1H-pyrazol-4-yl)-5-(2,4-dichlorophenyl)isoxazole-3-carboxamide;
4-Bromo-5-(2,4-dichlorophenyl)-N-(2-(2-fluorophenyl)-1,5-dimethyl-3-oxo-2,3-dihydro-1H-pyrazol-4-yl)isoxazole-3-carboxamide;
5-(2-Chloro-4-methoxyphenyl)-N-(2-cyclohexyl-1,5-dimethyl-3-oxo-2,3-dihydro-1H-pyrazol-4-yl)-4-cyclopropy lisoxazole-3-carboxamide;
N-(2-Cyclohexyl-1,5-dimethyl-3-oxo-2,3-dihydro-1H-pyrazol-4-yl)-4-cyclopropyl-5-(2,4-dichlorophenyl) isoxazole-3-carboxamide;
4-Cyclopropyl-5-(2,4-dichlorophenyl)-N-(2-(2-fluorophenyl)-1,5-dimethyl-3-oxo-2,3-dihydro-1H-pyrazol-4-yl)isoxazole-3-carboxamide;
4-Cyano-N-(2-cyclohexyl-1,5-dimethyl-3-oxo-2,3-dihydro-1H-pyrazol-4-yl)-5-(2,4-dichlorophenyl)isoxazole-3-carboxamide;
4-Cyano-5-(2,4-dichlorophenyl)-N-(2-(2-fluorophenyl)-1,5-dimethyl-3-oxo-2,3-dihydro-1H-pyrazol-4-yl)isoxazole-3-carboxamide;
N-(2-Cyclohexyl-1,5-dimethyl-3-oxo-2,3-dihydro-1H-pyrazol-4-yl)-5-(2,4-dichloro phenyl)-4-(trifluoro methyl)isoxazole-3-carboxamide;
5-(2,4-Dichlorophenyl)-N-(2-(2-fluorophenyl)-1,5-dimethyl-3-oxo-2,3-dihydro-1H-pyrazol-4-yl)-4-(trifluoromethyl)isoxazole-3-carboxamide;
5-(2-Chloro-4-(methoxymethyl)phenyl)-N-(2-cyclohexyl-1,5-dimethyl-3-oxo-2,3-dihydro-1H-pyrazol-4-yl)-4-methylisoxazole-3-carboxamide;
N-(2-Cyclohexyl-1,5-dimethyl-3-oxo-2,3-dihydro-1H-pyrazol-4-yl)-5-(4-methoxy-3-methylphenyl)-4-methylisoxazole-3-carboxamide;
5-(4-Chlorophenyl)-N-(2-cyclohexyl-1,5-dimethyl-3-oxo-2,3-dihydro-1H-pyrazol-4-yl)-4-methylisoxazole-3-carboxamide;
5-(2-Chloro-4-(trifluoromethyl)phenyl)-N-(2-cyclohexyl-1,5-dimethyl-3-oxo-2,3-dihydro-1H-pyrazol-4-yl)-4-methylisoxazole-3-carboxamide;
N-(2-Cyclohexyl-1,5-dimethyl-3-oxo-2,3-dihydro-1H-pyrazol-4-yl)-5-(2-methoxyphenyl)-4-methylisoxazole-3-carboxamide;
N-(2-Cyclohexyl-1,5-dimethyl-3-oxo-2,3-dihydro-1H-pyrazol-4-yl)-5-(2,4-difluorophenyl)-4-methylisoxazole-3-carboxamide;
5-(3-Chlorophenyl)-N-(2-cyclohexyl-1,5-dimethyl-3-oxo-2,3-dihydro-1H-pyrazol-4-yl)-4-methylisoxazole-3-carboxamide;
5-(Benzofuran-5-yl)-N-(2-cyclohexyl-1,5-dimethyl-3-oxo-2,3-dihydro-1H-pyrazol-4-yl)-4-methylisoxazole-3-carboxamide;
N-(2-Cyclohexyl-1,5-dimethyl-3-oxo-2,3-dihydro-1H-pyrazol-4-yl)-5-(2,3-difluorophenyl)-4-methylisoxazole-3-carboxamide;
N-(2-Cyclohexyl-1,5-dimethyl-3-oxo-2,3-dihydro-1H-pyrazol-4-yl)-5-(2,3-dihydrobenzofuran-7-yl)-4-methylisoxazole-3-carboxamide;
5-(4-Chloro-2-fluorophenyl)-N-(2-cyclohexyl-1,5-dimethyl-3-oxo-2,3-dihydro-1H-pyrazol-4-yl)-4-methylisoxazole-3-carboxamide;
5-(2-Chloro-4-fluorophenyl)-N-(2-cyclohexyl-1,5-dimethyl-3-oxo-2,3-dihydro-1H-pyrazol-4-yl)-4-methylisoxazole-3-carboxamide;
N-(2-Cyclohexyl-1,5-dimethyl-3-oxo-2,3-dihydro-1H-pyrazol-4-yl)-5-(2-fluorophenyl)-4-methylisoxazole-3-carboxamide;
N-(2-Cyclohexyl-1,5-dimethyl-3-oxo-2,3-dihydro-1H-pyrazol-4-yl)-5-(4-fluorophenyl)-4-methylisoxazole-3-carboxamide;
5-(4-Chloro-2-ethoxyphenyl)-N-(2-(2-fluorophenyl)-1,5-dimethyl-3-oxo-2,3-dihydro-1H-pyrazol-4-yl)-4-methylisoxazole-3-carboxamide;
5-(2,3-Dihydrobenzofuran-5-yl)-N-(2-(2-fluorophenyl)-1,5-dimethyl-3-oxo-2,3-dihydro-1H-pyrazol-4-yl)-4-methylisoxazole-3-carboxamide;
N-(2-Cyclohexyl-1,5-dimethyl-3-oxo-2,3-dihydro-1H-pyrazol-4-yl)-5-(2,5-dichlorophenyl)-4-methylisoxazole-3-carboxamide;
Ethyl 2-(3-((2-cyclohexyl-1,5-dimethyl-3-oxo-2,3-dihydro-1H-pyrazol-4-yl)carbamoyl)-4-methylisoxazol-5-yl)benzoate;
N-(2-Cyclohexyl-1,5-dimethyl-3-oxo-2,3-dihydro-1H-pyrazol-4-yl)-5-(2-fluoro-5-methoxyphenyl)-4-methylisoxazole-3-carboxamide;
5-(4-Chloro-2-methoxyphenyl)-N-(2-cyclohexyl-1,5-dimethyl-3-oxo-2,3-dihydro-1H-pyrazol-4-yl)-4-methylisoxazole-3-carboxamide;
N-(2-Cyclohexyl-1,5-dimethyl-3-oxo-2,3-dihydro-1H-pyrazol-4-yl)-5-(2,3-dichlorophenyl)-4-methylisoxazole-3-carboxamide;
5-(4-Chloro-2-ethoxyphenyl)-N-(2-cyclohexyl-1,5-dimethyl-3-oxo-2,3-dihydro-1H-pyrazol-4-yl)-4-methylisoxazole-3-carboxamide;
5-(4-Chloro-2-cyclopropylphenyl)-N-(2-cyclohexyl-1,5-dimethyl-3-oxo-2,3-dihydro-1H-pyrazol-4-yl)-4-methylisoxazole-3-carboxamide;
N-(2-Cyclohexyl-1,5-dimethyl-3-oxo-2,3-dihydro-1H-pyrazol-4-yl)-5-(2-methoxy-3-methylphenyl)-4-methylisoxazole-3-carboxamide;
N-(2-Cyclohexyl-1,5-dimethyl-3-oxo-2,3-dihydro-1H-pyrazol-4-yl)-5-(2-cyclopropyl-4-fluorophenyl)-4-methylisoxazole-3-carboxamide;
5-(2-Chloro-4-(cyclopropylmethoxy)phenyl)-N-(2-cyclohexyl-1,5-dimethyl-3-oxo-2,3-dihydro-1H-pyrazol-4-yl)-4-methylisoxazole-3-carboxamide;
5-(Benzofuran-7-yl)-N-(2-cyclohexyl-1,5-dimethyl-3-oxo-2,3-dihydro-1H-pyrazol-4-yl)-4-methylisoxazole-3-carboxamide;
N-(2-Cyclohexyl-1,5-dimethyl-3-oxo-2,3-dihydro-1H-pyrazol-4-yl)-5-(2,3-dihydrobenzo furan-5-yl)-4-methyl isoxazole-3-carboxamide;
5-(Chroman-8-yl)-N-(2-cyclohexyl-1,5-dimethyl-3-oxo-2,3-dihydro-1H-pyrazol-4-yl)-4-methyl isoxazole-3-carboxamide;
N-(2-Cyclohexyl-1,5-dimethyl-3-oxo-2,3-dihydro-1H-pyrazol-4-yl)-4-cyclopropyl-5-(4-methoxy-2-(trifluoro methyl)phenyl)isoxazole-3-carboxamide;
N-(2-Cyclohexyl-1,5-dimethyl-3-oxo-2,3-dihydro-1H-pyrazol-4-yl)-4-cyclopropyl-5-(2-fluoro-4-methoxyphenyl) isoxazole-3-carboxamide;
4-cyclopropyl-N-(2-(2-fluorophenyl)-1,5-dimethyl-3-oxo-2,3-dihydro-1H-pyrazol-4-yl)-5-(4-methoxy-2-(trifluoromethyl)phenyl)isoxazole-3-carboxamide;
5-(2-chloro-4-(trifluoromethyl)phenyl)-N-(2-(2-fluorophenyl)-1,5-dimethyl-3-oxo-2,3-dihydro-1H-pyrazol-4-yl)-4-methylisoxazole-3-carboxamide;
N-(2-(2-fluorophenyl)-1,5-dimethyl-3-oxo-2,3-dihydro-1H-pyrazol-4-yl)-5-(4-methoxy-3-methylphenyl)-4-methylisoxazole-3-carboxamide;
N-(2-(2-fluorophenyl)-1,5-dimethyl-3-oxo-2,3-dihydro-1H-pyrazol-4-yl)-5-(4-methoxy-2-methylphenyl)-4-methylisoxazole-3-carboxamide;
5-(4-chloro-2-methylphenyl)-N-(2-(2-fluorophenyl)-1,5-dimethyl-3-oxo-2,3-dihydro-1H-pyrazol-4-yl)-4-methylisoxazole-3-carboxamide;
5-(4-chlorophenyl)-N-(2-(2-fluorophenyl)-1,5-dimethyl-3-oxo-2,3-dihydro-1H-pyrazol-4-yl)-4-methylisoxazole-3-carboxamide;
5-(2,5-dichlorophenyl)-N-(2-(2-fluorophenyl)-1,5-dimethyl-3-oxo-2,3-dihydro-1H-pyrazol-4-yl)-4-methylisoxazole-3-carboxamide;
N-(2-(2-fluorophenyl)-1,5-dimethyl-3-oxo-2,3-dihydro-1H-pyrazol-4-yl)-5-(2-methoxy-3-methylphenyl)-4-methylisoxazole-3-carboxamide; and
5-(2-chloro-4-cyclopropylphenyl)-N-(2-(2,5-dimethylphenyl)-1,5-dimethyl-3-oxo-2,3-dihydro-1H-pyrazol-4-yl)-4-methylisoxazole-3-carboxamide;
   or a pharmaceutically acceptable salt thereof.

### Embodiment 19

A compound of formula I according to any preceding Embodiment, wherein the compound is selected from:
5-(2,4-Dichlorophenyl)-N-(2-(2-fluorophenyl)-1,5-dimethyl-3-oxo-2,3-dihydro-1H-pyrazol-4-yl)-4-methylisoxazole-3-carboxamide;
5-(2-Chloro-4-cyclopropylphenyl)-N-(2-(2-fluorophenyl)-1,5-dimethyl-3-oxo-2,3-dihydro-1H-pyrazol-4-yl)-4-methylisoxazole-3-carboxamide;
4-Chloro-5-(2-chloro-4-(difluoromethoxy)phenyl)-N-(2-(2-fluorophenyl)-1,5-dimethyl-3-oxo-2,3-dihydro-1H-pyrazol-4-yl)isoxazole-3-carboxamide;
N-(2-(2-fluorophenyl)-1,5-dimethyl-3-oxo-2,3-dihydro-1H-pyrazol-4-yl)-5-(4-methoxy-2-methylphenyl)-4-methylisoxazole-3-carboxamide;
5-(2-Chloro-4-cyclopropylphenyl)-N-(2-cyclohexyl-1,5-dimethyl-3-oxo-2,3-dihydro-1H-pyrazol-4-yl)-4-methylisoxazole-3-carboxamide; 5-(2-Chloro-4-(methoxymethyl)phenyl)-N-(2-cyclohexyl-1,5-dimethyl-3-oxo-2,3-dihydro-1H-pyrazol-4-yl)-4-methylisoxazole-3-carboxamide; 5-(2-Chloro-4-methoxy-phenyl)-4-methyl-isoxazole-3-carboxylic acid;
4-Bromo-5-(2-chloro-4-methoxyphenyl)-N-(2-(2-fluorophenyl)-1,5-dimethyl-3-oxo-2,3-dihydro-1H-pyrazol-4-yl)isoxazole-3-carboxamide;
5-(2-Chloro-4-cyclopropylphenyl)-N-(2-(2-fluorophenyl)-1-trideuteromethyl-5-methyl-3-oxo-2,3-dihydro-1H-pyrazol-4-yl)-4-methylisoxazole-3-carboxamide or a pharmaceutically acceptable salt or co-crystal thereof.

In another embodiment individual compounds according to the invention are those listed in the Examples section below.

As used herein, the term “isomers” refers to different compounds that have the same molecular formula but differ in arrangement and configuration of the atoms. Also as used herein, the term “an optical isomer” or “a stereoisomer” refers to any of the various stereo isomeric configurations which may exist for a given compound of the present invention and includes geometric isomers. It is understood that a substituent may be attached at a chiral center of a carbon atom. The term “chiral” refers to molecules which have the property of non-superimposability on their mirror image partner, while the term “achiral” refers to molecules which are superimposable on their mirror image partner. Therefore, the invention includes enantiomers, diastereomers or racemates of the compound. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain compounds described herein contain one or more asymmetric centers or axes and may thus give rise to enantiomers, diastereomers, and other stereoisomeric forms that may be defined, in terms of absolute stereochemistry, as (R)- or (S)-.

Depending on the choice of the starting materials and procedures, the compounds can be present in the form of one of the possible isomers or as mixtures thereof, for example as pure optical isomers, or as isomer mixtures, such as racemates and diastereoisomer mixtures, depending on the number of asymmetric carbon atoms. The present invention is meant to include all such possible isomers, including racemic mixtures, diasteriomeric mixtures and optically pure forms. Optically active (R)- and (S)-isomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

As used herein, the terms “salt” or “salts” refers to an acid addition or base addition salt of a compound of the invention. “Salts” include in particular “pharmaceutical acceptable salts”. The term “pharmaceutically acceptable salts” refers to salts that retain the biological effectiveness and properties of the compounds of this invention and, which typically are not biologically or otherwise undesirable.

Compounds of the invention, i.e. compounds of formula (I) or (Ia) that contain groups capable of acting as donors and/or acceptors for hydrogen bonds may be capable of forming co-crystals with suitable co-crystal formers. These co-crystals may be prepared from compounds of formula (I) or (Ia) by known co-crystal forming procedures. Such procedures include grinding, heating, co-subliming, co-melting, or contacting in solution compounds of formula (I) or (Ia) with the co-crystal former under crystallization conditions and isolating co-crystals thereby formed. Suitable co-crystal formers include those described in WO 2004/078163. Hence the invention further provides co-crystals comprising a compound of formula (I) or (Ia).

In many cases, the compounds of the present invention are capable of forming acid and/or base salts and or co-crystals by virtue of the presence of the carboxamide group or groups similar thereto.

Pharmaceutically acceptable acid addition salts or co-crystals can be formed with inorganic acids and organic acids.

Inorganic acids from which salts or co-crystals can be derived include, for example, hydrochloric acid, hydrobromic acid, sulfuric acid, nitric acid, phosphoric acid, and the like.

Organic acids from which salts or co-crystals can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, and the like.

Pharmaceutically acceptable base addition salts or co-crystals can be formed with inorganic and organic bases.

Inorganic bases from which salts or co-crystals can be derived include, for example, ammonium salts and metals from columns I to XII of the periodic table. In certain embodiments, the salts are derived from sodium, potassium, ammonium, calcium, magnesium, silver, and zinc; particularly suitable salts include ammonium, potassium, sodium, calcium and magnesium salts.

Organic bases from which salts or co-crystals can be derived include, for example, primary, secondary, and tertiary amines, substituted amines including naturally occurring substituted amines, cyclic amines, basic ion exchange resins, and the like. Certain organic amines include cholinate, lysine, meglumine, piperazine and tromethamine.

In another aspect, the present invention provides compounds of formula (I) or (Ia) in acetate, ascorbate, adipate, aspartate, benzoate, besylate, bromide/hydrobromide, bicarbonate/carbonate, bisulfate/sulfate, caprate, chloride/hydrochloride, citrate, ethandisulfonate, fumarate, gluceptate, gluconate, glucuronate, glutamate, glutarate, glycolate, hippurate, hydroiodide/iodide, isethionate, lactate, lactobionate, malate, maleate, malonate, mandelate, mesylate, methylsulphate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfate, tartrate, tosylate trifenatate, or xinafoate salt or co-crystal form.

In another aspect, the present invention provides compounds of formula (I) or (Ia) in sodium, potassium, ammonium, calcium, magnesium, silver, zinc, cholinate, lysine, meglumine, piperazine or tromethamine salt or co-crystal form.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F ³¹P, ³²P, ³⁵S, ³⁶Cl, ¹²⁵I respectively. The invention includes various isotopically labeled compounds as defined herein, for example those into which radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically-labeled compounds of formula I can generally be prepared by conventional techniques known to those skilled in the art or by processes analogous to those described in the accompanying Examples and Preparations using an appropriate isotopically-labeled reagents in place of the non-labeled reagent previously employed.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the formula (I). The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). For example, in another embodiment of the invention as described anywhere herein there is provided a compound of formula I wherein, R⁴ is deuterated C₁-C₆ alkyl, or deuterated C₁-C₆ alkoxy. In another Embodiment, R⁴ is deuteromethyl (—CD₃) or deuteromethoxy (—OCD₃); and R¹, R², R³, R⁵, n and m are as defined in any preceding Embodiment.

Pharmaceutically acceptable solvates in accordance with the invention include those wherein the solvent of crystallization may be isotopically substituted, e.g. D₂O, d₆-acetone, d₆-DMSO.

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The term “a therapeutically effective amount” of a compound of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc.

In one non-limiting embodiment, the term “a therapeutically effective amount” refers to the amount of the compound of the present invention that, when administered to a subject, is effective to (1) at least partially alleviating, inhibiting, preventing and/or ameliorating a condition, or a disorder or a disease (i) mediated by Smurf-1, or (ii) associated with Smurf-1 activity, or (iii) characterized by activity (normal or abnormal) of Smurf-1; or (2) reducing or inhibiting the activity of Smurf-1; or (3) reducing or inhibiting the expression of Smurf-1 or increasing Smurf-1 protein levels. In another non-limiting embodiment, the term “a therapeutically effective amount” refers to the amount of the compound of the present invention that, when administered to a cell, or a tissue, or a non-cellular biological material, or a medium, is effective to at least partially reducing or inhibiting the activity of Smurf-1; or at least partially reducing or inhibiting the expression of Smurf-1 or increasing Smurf-1 protein levels.

As used herein, the term “subject” refers to an animal. Typically the animal is a mammal. A subject also refers to for example, primates (e.g., humans, male or female), cows, sheep, goats, horses, dogs, cats, rabbits, rats, mice, fish, birds and the like. In certain embodiments, the subject is a primate. In yet other embodiments, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder.

As used herein, a subject is “in need of” a treatment if such subject would benefit biologically, medically or in quality of life from such treatment.

As used herein, the term “a,” “an,” “the” and similar terms used in the context of the present invention (especially in the context of the claims) are to be construed to cover both the singular and plural unless otherwise indicated herein or clearly contradicted by the context.

All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

Any asymmetric atom (e.g., carbon or the like) of the compound(s) of the present invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated double bonds may, if possible, be present in cis-(Z)- or trans-(E)-form.

Accordingly, as used herein a compound of the present invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound.

In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

Furthermore, the compounds of the present invention, including their salts, can also be obtained in the form of their hydrates, or include other solvents used for their crystallization. The compounds of the present invention may inherently or by design form solvates with pharmaceutically acceptable solvents (including water); therefore, it is intended that the invention embrace both solvated and unsolvated forms. The term “solvate” refers to a molecular complex of a compound of the present invention (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water. The compounds of the present invention, including salts, hydrates and solvates thereof, may inherently or by design form polymorphs.

The compounds of the invention may be synthesized by the following general process, specific examples of which are described in more detail in the Examples.

Thus, as a further aspect of the present invention, a compound of formula I may be prepared by reacting a compound of formula II

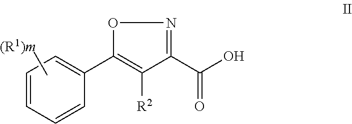

wherein m and the R groups are previously herein defined, with a compound of formula III

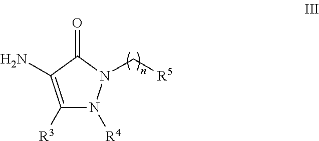

wherein n and the R groups are previously herein defined, by a standard amide coupling reaction well-known to those skilled in the art and including those conditions provided in the Examples.

A compound of formula II may be prepared by deprotection of a compound of formula IV

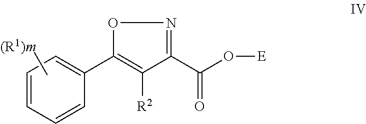

where R² represents H, halo, cyano, C₁-C₆ alkyl, C₁-C₆ haloalkyl, C₁-C₆ alkoxy, (C₁-C₆ alkoxy)C₁-C₆ alkyl-, C₁-C₆ haloalkoxy, C₃-C₇ cycloalkyl, (C₃-C₇ cycloalkyl)C₁-C₆ alkyl- or (C₃-C₇ cycloalkyl)C₁-C₆ alkoxy-, E is a suitable ester group, e.g. methyl; and the other groups are as defined above. Deprotection of the ester group may be carried out under standard conditions, e.g. sodium hydroxide in methanol at elevated temperature.

A compound of formula II, where R2 represents halo may be prepared by reaction of a compound of formula IV where R2 represents H, with a suitable electrophilic reagent, eg N-bromosuccinimide. Deprotection of the ester group may be carried out under standard conditions, e.g. sodium hydroxide in methanol at elevated temperature.

A compound of formula II, where R² represents C₁-C₆ alkyl, C₁-C₆ haloalkyl, C₁-C₆ alkoxy, (C₁-C₆ alkoxy)C₁-C₆ alkyl-, C₁-C₆ haloalkoxy, C₃-C₇ cycloalkyl, (C₃-C₇ cycloalkyl)C₁-C₆ alkyl- or (C₃-C₇ cycloalkyl)C₁-C₆ alkoxy-, may be prepared by a metal facilitated insertion reaction of a compound of formula IV, where R² represents halo, where the facilitating metal may be copper or palladium eg Copper iodide or Palladium acetate. Deprotection of the ester group may be carried out under standard conditions, e.g. sodium hydroxide in methanol at elevated temperature.

A compound of formula IV may be prepared by reaction of a compound of formula V

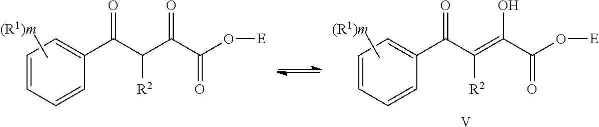

where R² represents H or methyl, E is a suitable ester group, e.g. methyl or ethyl, and the other groups are as defined above, with a suitable salt of hydroxylamine, e.g. the hydrochloride salt, in a suitable solvent such as methanol at elevated temperature

A compound of formula V may be prepared by reaction of a compound of formula VI

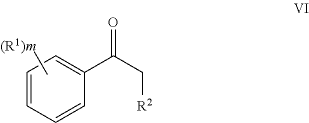

where R² is H, methyl or cyano, and the other substituents are defined as above, with dimethyl oxalate or diethyl oxalate and a suitable base, such as sodium methoxide, in a suspension in a suitable solvent, e.g. methanol. Alternatively, the compound of formula VI may be reacted with ethyl chlorooximidoacetate and a suitable base such as triethylamine in a suitable solvent eg ethanol.

Alternatively a compound of formula II may be prepared by reaction of a compound of formula VII

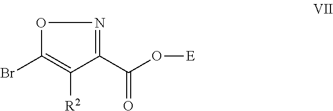

where R² is methyl and E is a suitable ester group, e.g. methyl or ethyl, with a suitable boronic acid/ester in a suitable solvent such as acetonitrile/water in a Suzuki type reaction under suitable conditions well-known to those skilled in the art, including those described in the Examples. Deprotection of the ester group may be carried out under standard conditions, e.g. sodium hydroxide in methanol at elevated temperature.

A compound of formula III may be prepared from a compound of formula VIII

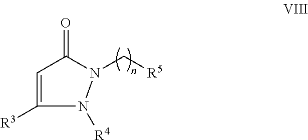

where the substituents are as previously defined, by nitration, e.g. by nitric acid in a suitable solvent such as trifluoroacetic acid followed by reduction, optionally in a one-pot reaction using a suitable reagent, such as iron powder.

A compound of formula VIII may be prepared from a compound of formula IX

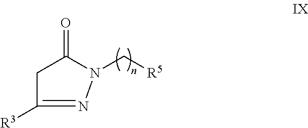

where the substituents are as previously defined, by alklyation with a compound of formula R⁴—Y, where Y is suitable halide, e.g. iodide, in a suitable solvent, e.g. dimethylformamide.

A compound of formula IX may be prepared by reaction of a compound of formula X

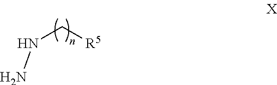

where R⁵ is as previously defined, with a compound of formula XI

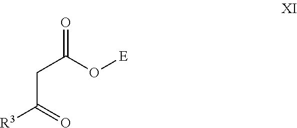

where R³ is as previously defined and E is a suitable ester group, e.g. ethyl, under suitable conditions, e.g. aqueous acetic acid at elevated temperature.

All starting materials, building blocks, reagents, acids, bases, dehydrating agents, solvents and catalysts utilized to synthesize the compounds of the present invention, including compounds of formula VII, X and XI, are either commercially available or can be produced by organic synthesis methods known to one of ordinary skill in the art (Houben-Weyl 4^{th} Ed. 1952, Methods of Organic Synthesis, Thieme, Volume 21).

The invention further includes any variant of the present processes, in which an intermediate product obtainable at any stage thereof is used as starting material and the remaining steps are carried out, or in which the starting materials are formed in situ under the reaction conditions, or in which the reaction components are used in the form of their salts or optically pure material.

Compounds of the invention and intermediates can also be converted into each other according to methods generally known to those skilled in the art.

Within the scope of this text, only a readily removable group that is not a constituent of the particular desired end product of the compounds of the present invention is designated a “protecting group”, unless the context indicates otherwise. The protection of functional groups by such protecting groups, the protecting groups themselves, and their cleavage reactions are described for example in standard reference works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/I, Georg Thieme Verlag, Stuttgart 1974, in H.-D. Jakubke and H. Jeschkeit, “Aminosauren, Peptide, Proteine” (Amino acids, Peptides, Proteins), Verlag Chemie, Weinheim, Deerfield Beach, and Basel 1982, and in Jochen Lehmann, “Chemie der Kohlenhydrate: Monosaccharide und Derivate” (Chemistry of Carbohydrates: Monosaccharides and Derivatives), Georg Thieme Verlag, Stuttgart 1974. A characteristic of protecting groups is that they can be removed readily (i.e. without the occurrence of undesired secondary reactions) for example by solvolysis, reduction, photolysis or alternatively under physiological conditions (e.g. by enzymatic cleavage).

Salts of compounds of the present invention having at least one salt-forming group may be prepared in a manner known to those skilled in the art. For example, salts of compounds of the present invention having acid groups may be formed, for example, by treating the compounds with metal compounds, such as alkali metal salts of suitable organic carboxylic acids, e.g. the sodium salt of 2-ethylhexanoic acid, with organic alkali metal or alkaline earth metal compounds, such as the corresponding hydroxides, carbonates or hydrogen carbonates, such as sodium or potassium hydroxide, carbonate or hydrogen carbonate, with corresponding calcium compounds or with ammonia or a suitable organic amine, stoichiometric amounts or only a small excess of the salt-forming agent preferably being used. Acid addition salts of compounds of the present invention are obtained in customary manner, e.g. by treating the compounds with an acid or a suitable anion exchange reagent. Internal salts of compounds of the present invention containing acid and basic salt-forming groups, e.g. a free carboxy group and a free amino group, may be formed, e.g. by the neutralisation of salts, such as acid addition salts, to the isoelectric point, e.g. with weak bases, or by treatment with ion exchangers.

Salts can be converted into the free compounds in accordance with methods known to those skilled in the art. Metal and ammonium salts can be converted, for example, by treatment with suitable acids, and acid addition salts, for example, by treatment with a suitable basic agent.

Mixtures of isomers obtainable according to the invention can be separated in a manner known to those skilled in the art into the individual isomers; diastereoisomers can be separated, for example, by partitioning between polyphasic solvent mixtures, recrystallisation and/or chromatographic separation, for example over silica gel or by e.g. medium pressure liquid chromatography over a reversed phase column, and racemates can be separated, for example, by the formation of salts with optically pure salt-forming reagents and separation of the mixture of diastereoisomers so obtainable, for example by means of fractional crystallisation, or by chromatography over optically active column materials.

Intermediates and final products can be worked up and/or purified according to standard methods, e.g. using chromatographic methods, distribution methods, (re-) crystallization, and the like.

The following applies in general to all processes mentioned herein before and hereinafter.

All the above-mentioned process steps can be carried out under reaction conditions that are known to those skilled in the art, including those mentioned specifically, in the absence or, customarily, in the presence of solvents or diluents, including, for example, solvents or diluents that are inert towards the reagents used and dissolve them, in the absence or presence of catalysts, condensation or neutralizing agents, for example ion exchangers, such as cation exchangers, e.g. in the H+ form, depending on the nature of the reaction and/or of the reactants at reduced, normal or elevated temperature, for example in a temperature range of from about −100° C. to about 190° C., including, for example, from approximately −80° C. to approximately 150° C., for example at from −80 to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

At all stages of the reactions, mixtures of isomers that are formed can be separated into the individual isomers, for example diastereoisomers or enantiomers, or into any desired mixtures of isomers, for example racemates or mixtures of diastereoisomers, for example analogously to the methods described under “Additional process steps”.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofuran or dioxane, liquid aromatic hydrocarbons, such as benzene or toluene, alcohols, such as methanol, ethanol or 1- or 2-propanol, nitriles, such as acetonitrile, halogenated hydrocarbons, such as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, methycyclohexane, or mixtures of those solvents, for example aqueous solutions, unless otherwise indicated in the description of the processes. Such solvent mixtures may also be used in working up, for example by chromatography or partitioning.

The compounds, including their salts, may also be obtained in the form of hydrates, or their crystals may, for example, include the solvent used for crystallization. Different crystalline forms may be present.

The invention relates also to those forms of the process in which a compound obtainable as an intermediate at any stage of the process is used as starting material and the remaining process steps are carried out, or in which a starting material is formed under the reaction conditions or is used in the form of a derivative, for example in a protected form or in the form of a salt, or a compound obtainable by the process according to the invention is produced under the process conditions and processed further in situ.

In another aspect, the present invention provides a pharmaceutical composition comprising a compound of the present invention and a pharmaceutically acceptable carrier. The pharmaceutical composition can be formulated for particular routes of administration such as oral administration, parenteral administration, and rectal administration, etc. In addition, the pharmaceutical compositions of the present invention can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifers and buffers, etc.

Typically, the pharmaceutical compositions are tablets or gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

Suitable compositions for oral administration include an effective amount of a compound of the invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in a mixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include an effective amount of a compound of the invention with a suitable carrier. Carriers suitable for transdermal delivery include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein a topical application may also pertain to an inhalation or to an intranasal application. They may be conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomizer or nebuliser, with or without the use of a suitable propellant.

Where the inhalable form of the active ingredient is an aerosol composition, the inhalation device may be an aerosol vial provided with a valve adapted to deliver a metered dose, such as 10 to 100 μl, e.g. 25 to 50 μl, of the composition, i.e. a device known as a metered dose inhaler.

Suitable such aerosol vials and procedures for containing within them aerosol compositions under pressure are well known to those skilled in the art of inhalation therapy. For example, an aerosol composition may be administered from a coated can, for example as described in EP-A-0642992. Where the inhalable form of the active ingredient is a nebulizable aqueous, organic or aqueous/organic dispersion, the inhalation device may be a known nebulizer, for example a conventional pneumatic nebulizer such as an airjet nebulizer, or an ultrasonic nebulizer, which may contain, for example, from 1 to 50 ml, commonly 1 to 10 ml, of the dispersion; or a hand-held nebulizer, sometimes referred to as a soft mist or soft spray inhaler, for example an electronically controlled device such as an AERx (Aradigm, US) or Aerodose (Aerogen), or a mechanical device such as a RESPIMAT (Boehringer Ingelheim) nebulizer which allows much smaller nebulized volumes, e.g. 10 to 100 μl, than conventional nebulizers. Where the inhalable form of the active ingredient is the finely divided particulate form, the inhalation device may be, for example, a dry powder inhalation device adapted to deliver dry powder from a capsule or blister containing a dry powder comprising a dosage unit of (A) and/or (B) or a multidose dry powder inhalation (MDPI) device adapted to deliver, for example, 3-25 mg of dry powder comprising a dosage unit of (A) and/or (B) per actuation. The dry powder composition preferably contains a diluent or carrier, such as lactose, and a compound that helps to protect against product performance deterioration due to moisture e.g. magnesium stearate. Suitable such dry powder inhalation devices include devices disclosed in U.S. Pat. No. 3,991,761 (including the AEROLIZER™ device), WO 05/113042, WO 97/20589 (including the CERTIHALER™ device), WO 97/30743 (including the TWISTHALER™ device) and WO 05/37353 (including the GYROHALER™ device).

When the composition comprises an aerosol formulation, it preferably contains, e.g., a hydro-fluoro-alkane (HFA) propellant, such as HFA134a or HFA227 or a mixture of these, and may contain one or more co-solvents known in the art, such as ethanol (up to 20% by weight), and/or one or more surfactants, such as oleic acid or sorbitan trioleate, and/or one or more bulking agents, such as lactose. When the composition comprises a dry powder formulation, it preferably contains, e.g., the compound of Formula I or pharmaceutical salts thereof having a particle diameter up to 10 microns, optionally together with a diluent or carrier, such as lactose, of the desired particle size distribution and a compound that helps to protect against product performance deterioration due to moisture, e.g., magnesium stearate. When the composition comprises a nebulised formulation, it preferably contains, e.g., the compound of Formula I or pharmaceutical salts thereof either dissolved, or suspended, in a vehicle containing water, a co-solvent, such as ethanol or propylene glycol and a stabilizer, which may be a surfactant.

The invention also includes (A) an agent of the invention in free form, or a pharmaceutically acceptable salt or solvate thereof, in inhalable form; (B) an inhalable medicament comprising such a compound in inhalable form together with a pharmaceutically acceptable carrier in inhalable form; (C) a pharmaceutical product comprising such a compound in inhalable form in association with an inhalation device; and (D) an inhalation device containing such a compound in inhalable form.

Dosages of agents of the invention employed in practising the present invention will of course vary depending, for example, on the particular condition to be treated, the effect desired and the mode of administration. In general, suitable daily dosages for administration by inhalation are of the order of 0.0001 to 30 mg/kg, typically 0.01 to 10 mg per patient, while for oral administration suitable daily doses are of the order of 0.01 to 100 mg/kg.

The present invention further provides anhydrous pharmaceutical compositions and dosage forms comprising the compounds of the present invention as active ingredients, since water may facilitate the degradation of certain compounds.

Anhydrous pharmaceutical compositions and dosage forms of the invention can be prepared using anhydrous or low moisture containing ingredients and low moisture or low humidity conditions. An anhydrous pharmaceutical composition may be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further provides pharmaceutical compositions and dosage forms that comprise one or more agents that reduce the rate by which the compound of the present invention as an active ingredient will decompose. Such agents, which are referred to herein as “stabilizers,” include, but are not limited to, antioxidants such as ascorbic acid, pH buffers, or salt buffers, etc.

The compounds of formula I and their pharmaceutically acceptable salts and solvates have the advantage that they are more selective, have a more rapid onset of action, are more potent, are better absorbed, are more stable, are more resistant to metabolism, have a reduced ‘food effect’, have an improved safety profile or have other more desirable properties (e.g. with respect to solubility or hygroscopicity) than the compounds of the prior art.

The compounds according to the invention in free form or in salt form, inhibit Smurf-1 selectively as indicated in in vitro and in vivo tests as provided in the next sections. The compounds according to the invention in free form or in salt form are useful in the treatment of conditions which respond to the inhibition of Smurf-1, such as for example, pulmonary arterial hypertension, fibrosis, rheumatoid arthritis, fracture healing, glaucoma, hereditary hemorrhagic telangiectasia (HHT), proteinuria, wound healing, COPD, and/or asthma.

Compounds of the invention are useful in the treatment of indications including:

Pulmonary Hypertension, including Pulmonary arterial hypertension (PAH)

Fibrosis

Rheumatoid Arthritis

Fracture healingGlaucoma

hereditary hemorrhagic telangiectasia (HHT)

proteinuria

wound healing

COPD

asthma

### Pulmonary Arterial Hypertension (PAH)

Pulmonary arterial hypertension has a multifactorial pathobiology. Vasoconstriction, remodeling of the pulmonary vessel wall and thrombosis contribute to increased pulmonary vascular resistance in PAH (Humbert et al, J. Am. Coll. Cardiol., 2004.). The compounds of the present invention disclosed herein are useful in the treatment of PAH and symptoms thereof. Pulmonary arterial hypertension shall be understood to encompass the following forms of pulmonary hypertension: idiopathic PAH (IPAH); heritable PAH (HPAH); PAH induced by drugs or toxins, PAH associated with other conditions (APAH), such as PAH associated with connective tissue diseases, PAH associated with HIV infection, PAH associated with portal hypertension, PAH associated with congenital heart diseases, PAH associated with schistosomiasis, PAH associated chronic haemolytic anaemia, or peristent pulmonary hypertension of the newborn (Galié et al, ERJ, 2009; Simonneau et al, JACC, 2009).

Idiopathic PAH refers to PAH of undetermined cause. Heritable PAH refers to PAH for which hereditary transmission is suspected or documented including those harboring mutations in the BMP receptor, BMPR2 or those with mutations in ALK1 or endoglin (with or without hereditary hemorrhagic talangiectasia).

PAH associated with drugs or toxins shall be understood to encompass PAH associated with ingestion of aminorex, a fenfluramine compound (e.g. fenfluramine or dexfenfluramine), certain toxic oils (e.g. rapeseed oil), pyrrolizidine alkaloids (e.g. bush tea), monocrotaline, amphetamines, L-tryptophan, methamphetamines, cocaine, phenylpropanolamine, St John's Wort, chemotherapeutic agents or SSRI's.

PAH associated with connective tissue diseases shall be understood to encompass PAH associated with systemic sclerosis, lung fibrosis, polymyositis, rheumatoid arthritis, Sjogren syndrome or PAH associated with systemic lupus erythematosis.

PAH associated with congenital heart diseases shall be understood to encompass patients with systemic to pulmonary shunts, PAH associated with Eisenmenger syndrome, small ventricular-septal or atrial-septal defects or PAH associated with corrective cardiac surgery.

PAH associated with chronic hemolytic anemia shall be understood to encompass patients with chronic hereditary and acquired anemias including patients with sickle cell disease, thalassemia, hereditary spherocytosis, stomatocytosis and microangiopathic hemolytic anemia.

Symptoms of PAH include dyspnea, angina, syncope and edema (McLaughlin et al., Circulation, 2006, 114:1417-1431). The compounds of the present invention disclosed herein are useful in the treatment of symptoms of PAH.

### Pulmonary Hypertension (PH)

Pulmonary hypertension (PH) shall be understood to be associated with the following conditions grouped according to the Dana Point clinical classification (Simonneau, G et al. JACCC, 2009):

Group 1′—PH shall be understood to be associated with patients harboring pulmonary veno-occlusive disease (PVOD) and pulmonary capillary hemangiomatosis (PCH).

Group 2—PH associated with left heart disease include those patients with left-sided ventricular or valvular diseases.

Group 3—PH as a result of lung diseases and/or hypoxia. Lung diseases resulting in PH shall be understood to encompass patients with pulmonary fibrosis, emphysema, combined pulmonary fibrosis and emphysema, bronchiectasis, cystic fibrosis and chronic obstructive lung disease (COPD).

Group 4—PH associated with chronic thromboembolism (CTEPH).

Group 5—PH associated with unclear or multifactoral etiologies. This category of PH patients shall be understood to encompass patients in one of the following groups: 1) chronic myeloproliferative disorders including polycythemia vera, essential thrombocythemia or chronic myeloid leukemia; 2) Systemic disorders including sarcoidosis, conditions resulting in destruction of the pulmonary capillary bed such as fibrosis, extrinsic compression of large pulmonary arteries, patients with Pulmonary Langerhan's cell histocytosis, lymphangioleiomyomatosis, neurofibromatosis type 1 and antineutrophil cytoplasmic antibodies-associated vasculitis; 3) Metabolic disorders including type Ia glycogen storage disease, deficiency of glucose-6-phosphatase, Gaucher disease and thyroid diseases (hypothyroidism and hyperthyroidism); 4) Encompassing patients with tumors that expand into the lumen of the pulmonary artery, occlusion of pulmonary microvasculature by metastatic tumor emboli, mediastinal fibrosis or patients with end-stage renal disease receiving long-term hemodialysis.

### Fibrosis

Dysregulation of the TGFβ/BMP signaling pathways have been shown to have a causative role in fibrosis of various organs including kidney, heart, lung, skin, pancreas and liver, as well as in systemic sclerosis and associated pathologies (as reviewed by Leask and Abraham, FASEB, 2004). It has been shown that BMP7 counteracts TGFβ1-induced epithelial-mesenchymal transition (EMT) (Zeisberg, M et al. Nat. Med, 2003) and collagen induction (Izumi, N et al. AJP. Lung, Cell, Mol., Physiol. 2005) both key mechanisms in the development of fibrosis. Direct evidence for a role of Smurf-1 in fibrotic pathologies was demonstrated in the unilateral ureteral obstruction (UUO) mouse model of progressive tubulointerstitial fibrosis of the kidney where enhanced levels of Smurf-1 were present in the diseased kidneys associated with decreased levels of the protective Smurf-1 substrate, Smad7 (Fukasawa, H et al. PNAS, 2004). More recently, a role for Smurf-1 in pulmonary fibrosis was suggested in data generated in pulmonary epithelial cells identifying a crucial role for the Smurf-1 substrate Smad7 in limiting EMT (Shukla, M A, et al. Am. J. Resp. Cell. Mol. Biol. 2009). The compounds of the present invention disclosed herein are useful in the treatment of fibrosis and symptoms thereof. Fibrosis shall be understood to encompass the following: patients with pulmonary fibrosis, idiopathic pulmonary fibrosis, cystic fibrosis, cirrhosis, endomyocardial fibrosis, mediastinal fibrosis, myelofibrosis, retroperitoneal fibrosis, progressive massive fibrosis, nephrogenic systemic fibrosis, Crohn's Disease, keloid, old myocardial infarction, scleroderma (systemic sclerosis), arthrofibrosis or adhesive capsulitis.

### Rheumatoid Arthritis

Pro-inflammatory cytokines such as tumor necrosis factor alpha (TNFα) play a key role in the onset and maintenance of chronic inflammatory conditions such as rheumatoid arthritis (RA). A reduction in bone density is commonly associated with RA and Smurf-1 has been shown to play a key role in mediating RA-induced bone loss. It was shown that TNFα triggered proteolytic degradation of the Smurf-1 substrates Smad1 and Runx2 both of which are essential for bone-forming osteoblast activity. Direct evidence in support of this link was demonstrated in smurf-1 KO mice where TNFα failed to impact osteoclast activity in bones from Smurf-1 KO mice but not those of corresponding wild-type mice (Guo, R et al. JBC, 2008). The compounds of the present invention disclosed herein are useful in the treatment of rheumatoid arthritis and symptoms thereof. RA shall be understood to encompass patients with chronic inflammation of the synovium secondary to swelling of synovial cells, excess synovial fluid and formation of fibrous tissue within joints. In addition, RA shall also encompass patients with RA due to a necrotizing granuloma, vasculitis, pyoderma gangrenosum, Sweet's syndrome, erythema nodosum, lobular panniculitis, atrophy of digital skin, palmar erythema or diffuse thinning of the skin. RA also extends to other organs and herein will encompass patients with fibrosis of the lungs, renal amyloidosis, atherosclerosis as a result of RA, pericarditis, endocarditis, left ventricular failure, valvulitis and fibrosis. RA will also encompass patients with ocular conditions of episcleritis and keratoconjunctivitis sicca, hematological disorders of warm autoimmune hemolytic anemia, neutropenia and thrmobocytosis, neurological conditions of peripheral neuropathy, mononeuritis multiplex and carpal tunnel syndrome, osteoporosis and lymphoma.

### Fracture Healing

The BMP pathway plays a role here and Smurf-1 inhibitors increase BMP signaling. The compounds of the present invention disclosed herein are useful in the treatment of fracture healing and symptoms thereof. Fracture healing shall be understood to encompass the technique of bone fracture repair whereby an endosteal impant containing pores into which osteoblasts and supporting connective tissue can migrate is surgically implanted at the site of bone fracture. The administration of inhibitors of Smurf-1 following insertion of the above described implant may aid integration of the implant and expedite recovery by enhancing proliferation of mesenchymal stem cells which differentate into osteoblasts (Zhao, M et al. JBC, 2004).

### Glaucoma

Elevated intraocular pressure (IOP) is one of the major risk factor for primary open angle glaucoma (POAG). IOP is maintained in anterior chamber by aqueous humor produced in ciliary body and outflowed through trabecular meshwork region. Increase aqueous humor outflow resistance associated with accumulation of extracellular matrix (ECM) deposition in trabecular meshwork region has been observed in glaucoma patients. This ECM pathology in POAG patients resembles fibrosis induced by TGFb proteins in many non-ocular systems. TGFb2 induced IOP increase was demonstrated in pre-clinical in vivo and ex vivo models. In several small scale clinical studies, the level of TGFb2 protein in aqueous humor has also been reported to be elevated in POAG patients. Modulating the TGFb activity in glaucoma patients could potentially lower IOP and lead to novel glaucoma therapies (Wordinger R J JOURNAL OF OCULAR PHARMACOLOGY AND THERAPEUTICS Volume 30, Number 2, 2014). In view of the role of Smurf1 in the regulation of TGFb signaling through its substrates BMP9 and SMAD 7 the compounds of the present invention (or their pharmaceutically acceptable salts) described herein would be useful in the treatment of Glaucoma.

### Hereditary Hemorrhagic Telangiectasia (HHT)

Hereditary Hemorrhagic Telangiectasia (HHT), also known as Osler-Weber-Rendu Syndrome, is a genetic disorder of the blood vessels affecting from 1:5000 to 1:40,000. A person with HHT has a tendency to form blood vessels that lack normal capillaries between an artery and vein, causing arterial blood under high pressure to flow directly into a vein, which may rupture and bleed. Symptoms of HHT may manifest as mild to severe, with 90-95% of patients experiencing nosebleeds by adulthood, 90-95% developing telangiectasias on the face or hands by middle age, and 40% developing lung arteriovenous malformations (AVM), which can pose significant risk. AVMs may also occur in the brain, liver, and intestine, with varying severity of health implications. HHT can be treated, most often with coagulation therapy, embolization, or surgical removal of affected tissue. HHT mutations cause haploinsufficiency in BMP signaling (Ricard et al. Blood, 2010) resulting in a vessel maturation defect and excessive branching of the vasculature which is in part, attributed to impaired BMP9 signaling (Choi, et al. PlosOne, 2013). Smurf1 down-regulates BMP signaling (Murakami Exp. Biol. Res. 2010 and Cao, et al. Sci. Rep. 2014) and has been reported to be expressed in the endothelial cells (Crose, et al. JBC, 2009 and Human Protein Atlas and GeneCards) and therefore, Smurf1 inhibitors may serve to restore BMP signaling and correct the angiogenesis abnormality. As such the compounds of the present invention (or their pharmaceutically acceptable salts) described herein would be useful in the treatment of HHT.

### Proteinuria

Abnormal amounts of protein in the urine are one of the earliest signs of chronic kidney disease which can result from hypertension, diabetes or diseases associated with inflammation in the kidneys. If left untreated, chronic kidney disease may progress to end-stage renal disease and kidney failure. Smurf1 is involved in multiple mechanisms associated with kidney function and proteinuria. The Smurf1 substrate Ras homolog gene family, member A (RhoA), plays a critical role in regulating the migration of kidney podocytes. Synaptopodin enables stress fiber formation within kidney podocytes by blocking the ability of Smurf1 to bind to and ubiquitinate RhoA thus promoting podocyte motility and modulation of sieving properties of the podocyte filtration barrier of the kidney (Asanuma, et al. Nat. Cell Biol. 2006). Additionally, the intracellular antagonist of transforming growth factor (TGF) β, Smad7 plays a key protective role in the kidney. Smurf1 activity has been shown to ubiquitinate and degrade Smad7 leading to tubulointerstitial fibrosis and kidney dysfunction (Fukasawa, et al. PNAS 2004). Together, these reports suggest that a Smurf1 inhibitor may enable podocyte migration and maintenance of the podocyte filtration barrier in addition to blocking propagation of pro-fibrotic signaling with the kidney ultimately providing therapeutic benefit for proteinuria. Accordingly the compounds of the invention (or their pharmaceutically acceptable salts) would be useful in the treatment of proteinuria.

### Wound Healing

Chronic non-healing wounds are most common in people over the age of 60 resulting in a significant amount of physical pain and are broadly classified into three groups: venous ulcers, diabetic and pressure ulcers. The precise timing of activity of the transforming growth factor (TGF) 1 and bone morphogenic protein (BMP) signaling pathways is essential in normal wound healing regulating key pro-healing processes of fibroblast migration and extracellular matrix deposition, inflammatory cell influx, angiogenesis and re-epithelialization (Pakyari, M et al. Adv. Wound Care 2013). Prolonged activation of TGF β may result in delayed wound healing and therapeutic intervention of established non-healing wounds with anti-TGF β antibodies results in improved healing and reduced scar hypertrophy (Lu et al. J. Am. Coll. Surg. 2005). Smurf1 regulates the extent of TGF β and BMP signaling (Murakami Exp. Biol. Res. 2010 and Cao, et al. Sci. Rep. 2014, Wang et al. J. Cell. Mol. Med. 2012) and therefore, it is anticipated that a Smurf1 inhibitor would normalized excessive of TGF β signaling enabling healing of chronic wounds. Accordingly the compounds of the invention (or their pharmaceutically acceptable salts) would be useful in the treatment of chronic non-healing wounds and/or wound healing generally.

### COPD and Asthma

Airway remodeling is evident in patients with chronic obstructive pulmonary disease (COPD) or asthma. The predominant features of airway remodeling in asthma are fibrosis, thickening of basement membrane, increased goblet cell numbers and enhanced smooth muscle cell mass with enhanced contractile response which are thought to be induced by chronic inflammation responsible for airway hyper-responsiveness and reversible airway obstruction (Carroll et al. Am. Rev Resp. Dis. 1993, Metcalfe, et al. Physiol. Rev. 1997 and Roche, et al. Lancet 1989). In COPD lung remodeling is characterized by disorganization of the epithelium in the large airways with squamous metaplasia, goblet cell hyperplasia and mucus hypersecretion, and small airway remodeling with expansion of smooth muscle, fibrosis and alveolar destruction in the development of emphysema ultimately resulting in restriction of airflow (De, Decramer, et al. Lancet, 2012, Pain et al. Eur. Respir. Rev. 2014 and Chung, Proc. Am. Thorac. Soc. 2005). In both diseases, there is evidence of down-regulated BMP signaling (Kariyawasam, et al. Am. J Resp. Crit. Care Med. 2008) and elevated TGF β (Mak. Et al. Respir. Med. 2009 and Chakir et al. J. All. Clin. Immunol. 2003) linked to pro-remodelling mechanism such as fibroblast-mesenchymal transition (Araya, et al. J. Clin. Invest. 2007), extracellular matrix deposition (Baarsma, et al. Am. J. Physiol. Lung Cell Mol. PHysiol. 2011) and inflammation (Chakir et al. J. All. Clin. Immunol. 2003). Smurf1 inhibitors may normalize TGF β signaling in critical pro-remodeling cells such as smooth muscle and fibroblasts and block progression of remodeling resulting in therapeutic benefit to COPD or asthma patients. Accordingly, the compounds of the invention (or their pharmaceutically acceptable salts) would be useful in the treatment of COPD and/or asthma.

Hence, the invention relates in a further aspect to compounds of Formula I and/or pharmaceutically acceptable salts thereof as defined in the first aspect for use in medicine.

Particularly, the compounds of the first aspect have valuable pharmacological properties, as described hereinbefore and hereinafter. The invention thus provides:
a compound of the first aspect as defined herein, as a pharmaceutical/for use in medicine;
a compound of the first aspect as defined herein, as a medicament/for use as a medicament;
a compound of the first aspect as defined herein, for the treatment of/for use in the treatment of disorders/diseases where Smurf-1 inhibitors have a beneficial effect; a compound of the first aspect as defined herein, for the treatment of/for use in the treatment of a disorder or disease selected from pulmonary hypertension, such as pulmonary arterial hypertension (PAH), fibrosis, rheumatoid arthritis, fracture healing, glaucoma, hereditary hemorrhagic telangiectasia (HHT), proteinuria, wound healing, COPD, and/or asthma;
a compound of the first aspect as defined herein, for the treatment of/for use in the treatment of pulmonary arterial hypertension (PAH), glaucoma, hereditary hemorrhagic telangiectasia (HHT), proteinuria, wound healing, COPD, and/or asthma;
the use of a compound of the first aspect as defined herein, for the manufacture of a medicament in the treatment of disorders/diseases where Smurf-1 inhibitors have a beneficial effect;
the use of a compound of the first aspect as defined herein, for the manufacture of a medicament for the treatment of a disorder or disease selected from pulmonary hypertension, such as pulmonary arterial hypertension (PAH), fibrosis, rheumatoid arthritis, fracture healing, glaucoma, hereditary hemorrhagic telangiectasia (HHT), proteinuria, wound healing, COPD, and/or asthma;
the use of a compound of the first aspect as defined herein, for the treatment of pulmonary hypertension, such as pulmonary arterial hypertension (PAH), fibrosis, rheumatoid arthritis, fracture healing, glaucoma, hereditary hemorrhagic telangiectasia (HHT), proteinuria, wound healing, COPD, and/or asthma;
a method for the treatment of disorders/diseases where Smurf-1 inhibitors have a beneficial effect comprising the step of administering to a subject a therapeutically effective amount of a compound of the first aspect as defined herein;
a method for the treatment of a disorder or disease selected from pulmonary hypertension, such as pulmonary arterial hypertension (PAH), fibrosis, rheumatoid arthritis, fracture healing, glaucoma, hereditary hemorrhagic telangiectasia (HHT), proteinuria, wound healing, COPD, and/or asthma comprising the step of administering to a subject a therapeutically effective amount of a compound of the first aspect as defined herein;
a method of modulating Smurf-1 receptor activity in a subject, comprising the step of administering to a subject a therapeutically effective amount of a compound of the first aspect as defined herein.

The invention also provides the known compounds N-(2,3-dihydro-1,5-dimethyl-3-oxo-2-phenyl-2,3-dihydro-1H-pyrazol-4-yl)-5-(2-fluorophenyl)isoxazole-3-carboxamide, N-(2,3-dihydro-1,5-dimethyl-3-oxo-2-phenyl-2,3-dihydro-1H-pyrazol-4-yl)-5-(4-fluorophenyl)isoxazole-3-carboxamide, N-(2,3-dihydro-1,5-dimethyl-3-oxo-2-phenyl-2,3-dihydro-1H-pyrazol-4-yl)-5-(4-ethoxyphenyl)isoxazole-3-carboxamide, N-(2,3-dihydro-1,5-dimethyl-3-oxo-2-phenyl-2,3-dihydro-1H-pyrazol-4-yl)-5-(3-chlorophenyl)isoxazole-3-carboxamide, N-(2,3-dihydro-1,5-dimethyl-3-oxo-2-phenyl-2,3-dihydro-1H-pyrazol-4-yl)-5-(4-bromophenyl)isoxazole-3-carboxamide, N-(2,3-dihydro-1,5-dimethyl-3-oxo-2-phenyl-2,3-dihydro-1H-pyrazol-4-yl)-5-(3,4-dimethoxyphenyl)isoxazole-3-carboxamide, N-(2,3-dihydro-1,5-dimethyl-3-oxo-2-phenyl-2,3-dihydro-1H-pyrazol-4-yl)-5-(3,4-dimethylphenyl)isoxazole-3-carboxamide, N-(2,3-dihydro-1,5-dimethyl-3-oxo-2-phenyl-2,3-dihydro-1H-pyrazol-4-yl)-5-(4-methylphenyl)isoxazole-3-carboxamide, N-(2,3-dihydro-1,5-dimethyl-3-oxo-2-phenyl-2,3-dihydro-1H-pyrazol-4-yl)-5-(3,4-dichlorophenyl)isoxazole-3-carboxamide, N-(2,3-dihydro-1,5-dimethyl-3-oxo-2-phenyl-2,3-dihydro-1H-pyrazol-4-yl)-5-(4-chlorophenyl)isoxazole-3-carboxamide, N-(2,3-dihydro-1,5-dimethyl-3-oxo-2-phenyl-2,3-dihydro-1H-pyrazol-4-yl)-5-(4-methoxyphenyl)isoxazole-3-carboxamide, N-(2,3-dihydro-1,5-dimethyl-3-oxo-2-phenyl-2,3-dihydro-1H-pyrazol-4-yl)-5-(3,4-difluorophenyl)isoxazole-3-carboxamide N-(2,3-dihydro-1,5-dimethyl-3-oxo-2-phenyl-1H-pyrazol-4-yl)-5-phenyl-isoxazole-3-carboxamide, N-(2,3-dihydro-1,5-dimethyl-3-oxo-2-phenyl-1H-pyrazol-4-yl)-5-(2,5-dimethoxyphenyl)-isoxazole-3-carboxamide, N-(2,3-dihydro-1,5-dimethyl-3-oxo-2-phenyl-1H-pyrazol-4-yl)-5-(3-methoxyphenyl)-isoxazole-3-carboxamide and N-(2,3-dihydro-1,5-dimethyl-3-oxo-2-phenyl-1H-pyrazol-4-yl)-5-(2-methoxyphenyl)-isoxazole-3-carboxamide, as a pharmaceutical/for use in medicine; as a medicament/for use as a medicament; for the treatment of/for use in the treatment of disorders/diseases where Smurf-1 inhibitors have a beneficial effect; for the treatment of/for use in the treatment of a disorder or disease selected from pulmonary hypertension, such as pulmonary arterial hypertension (PAH), fibrosis, rheumatoid arthritis, and fracture healing; for the treatment of/for use in the treatment of pulmonary hypertension, such as pulmonary arterial hypertension (PAH); for the manufacture of a medicament in the treatment of disorders/diseases where Smurf-1 inhibitors have a beneficial effect; for the manufacture of a medicament for the treatment of a disorder or disease selected from pulmonary hypertension, such as pulmonary arterial hypertension (PAH), fibrosis, rheumatoid arthritis, and fracture healing; for the treatment of pulmonary hypertension, such as pulmonary arterial hypertension (PAH), fibrosis, rheumatoid arthritis, and fracture healing.

The Smurf-1 inhibitors of formula I and/or their pharmaceutically acceptable salt(s) are also useful as co-therapeutic agents for use in combination with second agents.

The compounds of the present invention and/or their pharmaceutically acceptable salt(s) may be administered either simultaneously with, or before or after, one or more other therapeutic agents. The compound of the present invention may be administered separately, by the same or different route of administration, or together in the same pharmaceutical composition as the other agents.

In one embodiment, the invention provides a product comprising a compound of formula I and at least one other therapeutic agent as a combined preparation for simultaneous, separate or sequential use in therapy. In one embodiment, the therapy is the treatment of a disease which may be treated by inhibition of Smurf-1. Products provided as a combined preparation include a composition comprising the compound of formula I and the other therapeutic agent(s) together in the same pharmaceutical composition, or the compound of formula I and the other therapeutic agent(s) in separate form, e.g. in the form of a kit.

In one embodiment, the invention provides a pharmaceutical composition comprising a compound of formula I and another therapeutic agent(s). Optionally, the pharmaceutical composition may comprise a pharmaceutically acceptable excipient, as described above.

In one embodiment, the invention provides a kit comprising two or more separate pharmaceutical compositions, at least one of which contains a compound of formula (I). In one embodiment, the kit comprises means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

The pharmaceutical composition or combination of the present invention can be in unit dosage of about 1-1000 mg of active ingredient(s) for a subject of about 50-70 kg, or about 1-500 mg or about 1-250 mg or about 1-150 mg or about 0.5-100 mg, or about 1-50 mg of active ingredients. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present invention can be applied in vitro in the form of solutions, e.g., aqueous solutions, and in vivo either enterally, parenterally, advantageously intravenously, e.g., as a suspension or in aqueous solution. The dosage in vitro may range between about 10⁻³ molar and 10⁻⁹ molar concentrations. A therapeutically effective amount in vivo may range depending on the route of administration, between about 0.1-500 mg/kg, or between about 1-100 mg/kg.

### Pharmaceutical Assay

Compounds of the invention and their pharmaceutically acceptable salts, hereinafter referred to alternatively as “agents of the invention”, are useful as pharmaceuticals. In particular, the compounds are selective Smurf-1 inhibitors, and may be tested in the following assays.

To determine the HECT E3 ligase selectivity of the compounds, a panel of biochemical HECT E3 ligase autoubiquitinylation assays was employed (Smurf-1, Smurf-2, WWP1, WWP2, ITCH, Nedd4, Nedd4L and E6AP). The conjugation of ubiquitin to a protein substrate is a multistep process. In an initial ATP-requiring step, a thioester bond is formed between the carboxyl terminus of ubiquitin and an internal cystein residue of the ubiquitin-activating enzyme (E1). Activated ubiquitin is then transferred to a specific cystein residue of an ubiquitin-conjugating enzyme (E2). E2s donate ubiquitin to a HECT E3 ligase (E3) from which it is transferred to the substrate protein. HECT E3 ligases can auto-ubiquitinylate. This event is detected in the TR-FRET (Time-Resolved Fluorescence Resonance Energy Transfer) assay used in this panel. The reaction mix contains E1, E2, tagged-E3, biotin-conjugated ubiquitin, the compound and ATP in a suitable buffer and is incubated for 45 minutes to allow auto-ubiquitinylation of the E3 ligase. To measure the extent of ubiquitinylated E3 ligase by TR-FRET, the donor fluorophore Europium cryptate (Eu3+ cryptate), conjugated to streptavidin which subsequently binds to biotinylated ubiquitin, and the modified allophycocyanin XL665 (HTRF® primary acceptor fluorophore) coupled to a tag-specific antibody (HA, His or GST), which recognizes the respective E3 ligase fusion proteins, are added after the reaction is complete. When these two fluorophores are brought together by a biomolecular interaction (in this case ubiquitinylation of the E3 ligase), a portion of the energy captured by the Cryptate during excitation is released through fluorescence emission at 620 nm, while the remaining energy is transferred to XL665. This energy is then released by XL665 as specific fluorescence at 665 nm. Light at 665 nm is emitted only through FRET with Europium. Because Europium Cryptate is present in the assay, light at 620 nm is detected even when the biomolecular interaction does not bring XL665 within close proximity.

Autoubiquitinylation of Smurf-1 in cells leads to the proteasomal degradation of Smurf-1. Therefore, inhibition of the Smurf-1 catalytic domain abolishes Smurf-1 autoubiquitinylation and degradation, leading to accumulation of inhibited Smurf-1 protein in the cell.

Cellular activity of compounds at the Smurf-1 HECT domain is assessed by measuring the accumulation of Smurf-1 protein in HEK293 cells stably expressing Prolabel-tagged Smurf-1 under the control of a tetracycline-inducible promoter, using the DiscoverX PathHunter ProLabel Detection Kit. This technology measures the amount of Prolabel-tagged Smurf-1 in an enzyme complementation assay of the cell lysate. In this approach, a small 4 kDa complementing fragment of beta-galactosidase, called ProLabel, is expressed as an N-terminal fusion with human Smurf-1. This tag is the enzyme donor (ED) and enables detection of target protein levels after complementation with the larger portion of beta-galactosidase, termed EA for enzyme acceptor, to form functional beta-galactosidase enzyme. EA is exogenously added to the cell lysates. The enzyme activity is measured using a chemiluminescent substrate and is proportional to the amount of reconstituted enzyme and hence Smurf-1 levels.

Test and reference compounds are prepared at 180× [final] in 90% DMSO, and diluted 1:3 in 90% DMSO.

For the biochemical assay panel, 50 nl of the test compounds, reference compounds and buffer/DMSO control are transferred to the respective wells of a 384-well white GREINER “SMALL VOLUME” PS plate. The assay panel is run at room temperature on a Biomek FX liquid handling workstation. To the assay plates containing 50 nl compound or control solutions in 90% DMSO, 4.5 ul of E3 ligase solution were added per well, followed by 4.5 ul of the pre-incubated E1/E2/Ub mix or the pre-diluted ubiquitin (LOW control). Plates are shaken vigorously after each addition. In this assay the compound concentrations range from 3 nM to 10 uM in an 8-point dose-response curve.

After 45 min of incubation the ubiquitinylation reactions were stopped by adding 4.5 ul 2 mM NEM, immediately followed by 4.5 ul of a detection solution including the XL665-labeled antibody and the streptavidin-coupled europium to give a total volume of 18 ul. After an incubation time of 45 min in the dark, the plates are transferred into the Pherastar fluorescence reader to measure the TR-FRET signal.

For the cellular assay 250 nl of the test compounds, reference compounds and buffer/DMSO control are then transferred to the respective wells of a sterile 120 ul 384-well white GREINER PS, CELLSTAR, uClear tissue culture plate. To distribute the compound solution evenly in the medium before adding the cells, 10 ul of cell culture medium are added to each well of the compound containing plate using the MULTIDROP 384 dispenser and shaken vigorously. Cells are detached from the flask after a short incubation with trypsin-EDTA, counted and diluted to a concentration of 1.5×10⁶ cells/ml in culture medium. The expression of Smurf-1 is induced by adding doxycyline to a final concentration of 0.2 ug/ml. 10 ul of the cell suspension are added to each well of the compound-containing plates by using the MULTIDROP 384 dispenser. The plates are incubated over night at 37° C., 5% CO₂. In this assay the compound concentrations range from 6.75 nM to 22.5 uM in an 8-point dose-response curve.

After overnight incubation with the compounds the levels of Smurf-1 are determined using the PathHunter Prolabel detection kit from DiscoverX. First 10 ul of a lysis/CL detection working solution are added manually using a multi-channel step-pipettor, followed by the addition of 5 ul enzyme acceptor EA. The plates are mixed on a plate shaker and incubated for 2-3 hours at room-temperature before measuring the chemiluminescent signal in the PherStar plate reader. Compounds of the Examples, herein below, have Smurf-1 and Smurf-2 IC₅₀ values in the data measurements described above as shown in Table A.

TABLE A

| Example | Smurf-1/IC50 μM | Smurf-2/IC50 μM |
|---|---|---|
| 001 | 0.0295 | >10 |
| 002 | 0.0134 | 3.658 |
| 003 | 0.0543 | 16.75 |
| 006 | 0.0235 | 10.8 |
| 008 | 0.8825 | >10 |
| 010 | 0.0227 | 2.745 |
| 011 | 0.7775 | >10 |
| 012 | 1.0475 | >10 |
| 013 | 0.1025 | >20 |
| 019 | 0.0035 | 3.1 |
| 021 | 0.81 | >10 |
| 022 | 1.1625 | >10 |
| 023 | 0.0024 | 4.25 |
| 030 | 0.0133 | 5.4 |
| 033 | 0.22 | >20 |
| 034 | 0.0037 | 5.2333 |
| 037 | 0.036 | 17.75 |
| 039 | 0.0083 | 7.5 |
| 042 | 0.45 | 19.2 |
| 043 | 0 0033 | 5 4 |
| 046 | 4.15 | >10 |
| 47 | 0.0415 | >20.2 |
| 048 | 0.0077 | 2.4 |
| 049 | 0.0235 | 6.75 |
| 051 | 0.56 | >20.1 |
| 054 | 0.0108 | 5 |
| 055 | 0.109 | >20.2 |
| 062 | 0.965 | >20.2 |
| 057 | 0.024 | 7.5 |
| 069 | 0.0050 | 3.2 |
| 070 | 0.033 | 17.75 |
| 072 | 0.098 | >20.2 |
| 075 | 0.895 | >20.2 |
| 083 | 2.3 | >20.2 |
| 084 | 7.15 | >20.2 |
| 085 | 0.0505 | >20.2 |
| 086 | 0.26 | >20.2 |

The invention is illustrated by the following Examples.

### EXAMPLES

### General Conditions

Mass spectra were acquired on LC-MS, SFC-MS, or GC-MS systems using electrospray, chemical and electron impact ionization methods from a range of instruments of the following configurations: Agilent 1100 HPLC systems with an Agilent 6110 Mass Spectrometer, or Micromass Platform Mass Spectrometer or Thermo LTQ Mass Spectrometer; a Waters Acquity UPLC system with SQD Mass Spectrometer, a Waters FractionLynx HPLC system with 3100 Mass Spectrometer, a Waters UPC2 system with TQD Mass Spectrometer or a Waters Prep100 SFC-MS system with SQD2 Mass Spectrometer. [M+H]+ refers to protonated molecular ion of the chemical species.

NMR spectra were run on Bruker AVANCE 400 MHz, 500 MHz or Varian NMR spectrometers using ICON-NMR, under TopSpin program control. Spectra were measured at 298K, unless indicated otherwise, and were referenced relative to the solvent resonance.

The following examples are intended to illustrate the invention and are not to be construed as being limitations thereon.

It should be understood that the organic compounds according to the preferred embodiments may exhibit the phenomenon of tautomerism. As the chemical structures within this specification can only represent one of the possible tautomeric forms, it should be understood that the preferred embodiments encompasses any tautomeric form of the drawn structure.

Temperatures are given in degrees centigrade. If not mentioned otherwise, all evaporations are performed under reduced pressure, preferably between about 20-400 mbar. The structure of final products, intermediates and starting materials is confirmed by standard analytical Methods, e.g., microanalysis and spectroscopic characteristics, e.g., MS, IR, NMR. Abbreviations used are those conventional in the art. If not defined, the terms have their generally accepted meanings.

### Abbreviations:

ACN acetonitrile
BOP benzotriazolyloxytris[dimethylamino]-phosphoniumhexafluorophosphate
DCM dichloromethane
DIPEA diisopropylethylamine
DMF N,N-dimethylformamide
DMSO dimethylsulfoxide
EDC 1-ethyl-3-(3-dimethylaminopropyl)carbodiimide
EtOAc ethyl acetate
HATU O-(7-azabenzotriazol-1-yl)-N,N,N′,N′-tetramethyluronium-hexafluorophosphate
HMPA hexamethylphorphoramide
H or hr hour(s)
HPLC high pressure liquid chromatography
LC-MS liquid chromatography and mass spectrometry
LiHMDS lithium hexamethyl disilazane
M molar concentration
MeOH methanol
MS mass spectrometry
min minute(s)
ml milliliter(s)
m/z mass to charge ratio
PS polymer supported
RT room temperature
Rt retention time
T3P Propylphosphonic Anhydride TBTU benzotriazol-1-yl-N-tetramethyl-uronium tetrafluoroborate
TEA triethylamine
   TFA trifluoroacetic acid
   THF tetrahydrofuran

Referring to the examples that follow, compounds of the preferred embodiments were synthesized using the methods described herein, or other methods, which are known in the art.

The various starting materials, intermediates, and compounds of the preferred embodiments may be isolated and purified, where appropriate, using conventional techniques such as precipitation, filtration, crystallization, evaporation, distillation, and chromatography. Unless otherwise stated, all starting materials are obtained from commercial suppliers and used without further purification. Salts may be prepared from compounds by known salt-forming procedures.

It should be understood that the organic compounds according to the preferred embodiments may exhibit the phenomenon of tautomerism. As the chemical structures within this specification can only represent one of the possible tautomeric forms, it should be understood that the preferred embodiments encompasses any tautomeric form of the drawn structure.

If not indicated otherwise, the analytical HPLC conditions are as follows:

### Method A

System Agilent 1100 Series including Agilent MS1946D with chemical ionization
Column: Waters Symmetry C8 3.5 μm 2×50 mm,
Column Temperature: 50° C.
Eluents A: H₂O, containing 0.1% TFA
   B: acetonitrile, containing 0.1% TFA
Flow Rate: 1.0 ml/min
Gradient 10% to 95% B in 2 min

### Method B

System: Waters Acquity UPLC including Acquity SQD with electrospray ionization
Column: Waters Acquity HSS T3 1.8 μm 2.1×50 mm
Column Temperature: 50° C.
Eluents A: H₂O, containing 0.1% formic acid
   B: acetonitrile containing 0.1% formic acid
Flow Rate: 1.2 ml/min
Gradient 10% to 95% B in 2.5 min
   Method 2minHighpH
Column: Waters Acquity CSH 1.7 μm, 2.1×50 mm
Temperature: 50° C.
Mobile Phase: A: Water+0.1% Ammonia B: Acetonitrile+0.1% Ammonia
Flow rate: 1.0 mL/min
Gradient: 0.0 min 5% B, 0.2-1.3 min 5-98% B, 1.3-1.55 min 98% B, 1.55-1.6 min 98-5% B
   Method 2minLowpH
Column: Waters Acquity CSH 1.7 μm, 2.1×50 mm
Temperature: 50° C.
Mobile Phase: A: Water+0.1% Formic Acid B: Acetonitrile+0.1% Formic Acid
Flow rate: 1.0 mL/min
Gradient: 0.0 min 5% B, 0.2-1.3 min 5-98% B, 1.3-1.55 min 98% B, 1.55-1.6 min 98-5% B
   Method 2minlowpHv02
Column: Waters Acquity CSH 1.7 μm, 2.1×50 mm
Temperature: 50° C.
Mobile Phase: A: Water+0.1% TFA B: Acetonitrile+0.1% TFA
Flow rate: 1.0 mL/min
Gradient: 0.0 min 5% B, 0.2-1.55 min 5-98% B, 1.55-1.75 min 98% B, 1.75-1.8 min 98-5% B
   Method 2minLowpH_TFA
Column: Waters Acquity CSH 1.7 μm, 2.1×50 mm
Temperature: 50° C.
Mobile Phase: A: Water+0.1% Formic Acid B: Acetonitrile+0.1% TFA
Flow rate: 1.0 mL/min
Gradient: 0.0 min 5% B, 0.2-1.3 min 5-98% B, 1.3-1.55 min 98% B, 1.55-1.6 min 98-5% B
   Method 10minLowpH
Column: Waters Acquity CSH 1.7 μm, 2.1×100 mm
Temperature: 50° C.
Mobile Phase: A: Water+0.1% Formic Acid B: Acetonitrile+0.1% Formic Acid
Flow rate: 0.7 mL/min
Gradient: 0.0 min 2% B, 0.5-8.0 min 2-98% B, 8.0-9.0 min 98% B, 9.0-9.1 min 98-2% B

### Preparation of Final Compounds

### Example 001

### 5-(2,4-Dichlorophenyl)-N-(2-(2-fluorophenyl)-1,5-dimethyl-3-oxo-2,3-dihydro-1H-pyrazol-4-yl)-4-methylisoxazole-3-carboxamide

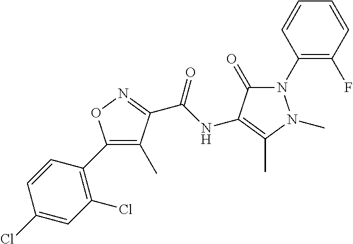 [SMILES: Cc1c(C(=O)Nc2c(C)n(C)n(-c3ccccc3F)c2=O)noc1-c1ccc(Cl)cc1Cl]

DMF (0.031 ml, 0.404 mmol) in DCM (2 ml) was treated with oxalyl chloride (0.019 ml, 0.222 mmol) at 0-5° C. To this solution, 5-(2,4-dichlorophenyl)-4-methylisoxazole-3-carboxylic acid (55 mg, 0.202 mmol) was added. After 10 min, 4-amino-2-(2-fluorophenyl)-1,5-dimethyl-1H-pyrazol-3(2H)one (commercially available)(44.7 mg, 0.202 mmol) and TEA (0.085 ml, 0.606 mmol) were added. After 5 min, the mixture was diluted with DCM and washed with water. The organic layer was dried over MgSO₄ and evaporated to dryness. The crude product was purified by preparative HPLC (Gilson). Column: Waters SunFire C18, 5 um, 30×100 mm. Solvent A: water+0.1% TFA, solvent B: acetonitrile. Gradient: 5-100% ACN in 20 min, 30 ml/min.

LC-MS: Rt 2.14 min; m/z 475 [M+H]⁺; Method A, ¹H-NMR (400 MHz, CDCl3) δ 8.24 (s, br, 1H) 7.57 (m, 1H) 7.36-7.49 (m, 4H) 7.24-7.31 (m, 2H) 3.19 (s, 3H) 2.35 (s, 3H) 2.22 (s, 3H).

### Example 002

### 5-(2-Chloro-4-cyclopropylphenyl)-N-(2-(2-fluorophenyl)-1,5-dimethyl-3-oxo-2,3-dihydro-1H-pyrazol-4-yl)-4-methylisoxazole-3-carboxamide

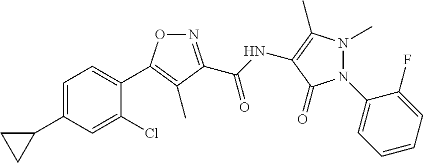 [SMILES: Cc1c(C(=O)Nc2c(C)n(C)n(-c3ccccc3F)c2=O)noc1-c1ccc(C2CC2)cc1Cl]

DMF (0.080 ml, 1.037 mmol) in DCM (4 ml) was treated with oxalyl chloride (0.061 ml, 0.691 mmol) at RT. To this solution, 5-(2-chloro-4-cyclopropylphenyl)-4-methylisoxazole-3-carboxylic acid (120 mg, 0.346 mmol) was added. After 10 min, 4-amino-2-(2-fluorophenyl)-1,5-dimethyl-1H-pyrazol-3(2H)one (commercially available) (76 mg, 0.346 mmol) and TEA (0.241 ml, 1.728 mmol) were added. After 5 min, the mixture evaporated to dryness. Purification by preparative HPLC (Gilson): Column: Waters SunFire C18, 5 um, 30×100 mm, solventA: Water+0.1% TFA, solventB: Acetonitrile Gradient: 5-100% ACN in 20 min, 30 ml/min. The product fraction was evaporated with diethyl ether to give a white foam.

LC-MS: Rt 2.12 min; m/z 481 [M+H]⁺; Method A, ¹H-NMR (400 MHz, CDCl₃) δ 8.21 (s, br, 1H) 7.38-7.47 (m, 2H) 7.25-7.31 (m, 3H) 7.20 (m, 1H) 7.07 (dd, 1H) 3.18 (s, 3H) 2.34 (s, 3H) 2.21 (s, 3H) 1.90-1.97 (m, 1H) 1.04-1.10 (m, 2H) 0.74-0.80 (m, 2H).

Alternatively, this compound may be prepared according to the following procedure:

To a stirred solution of 5-(2-chloro-4-cyclopropylphenyl)-4-methylisoxazole-3-carboxylic acid (22.0 g, 79.0 mmol) and 4-amino-2-(2-fluorophenyl)-1,5-dimethyl-1H-pyrazol-3(2H)one (commercially available) (17.53 g, 79.0 mmol) in EtOAc (250 ml) under nitrogen was added triethylamine (27.6 ml, 198 mmol) dropwise over 15 mins. The reaction mixture was cooled to 10° C. and T3P® (50% w/w solution in EtOAc, 70.7 ml, 119 mmol) was added via dropping funnel over 30 mins keeping the internal temperature below 10° C. The reaction mixture was allowed to warm to RT and stirred for 1 hr and a yellow precipitate formed. The slurry was cooled to 0° C. and water (300 ml) added over 30 mins. After warming to RT the resulting slurry was stirred for 1 hr then filtered washing with water (200 ml). The crude solid was dried in vacuo at 40° C. for 16 hrs and then slurried in MeOH (152 mL) and heated to 55° C. for 3 hrs. After cooling to RT the solid was collected by filtration washing with MeOH (15 ml) and dried in vacuo at 40° C. for 16 hrs to yield the title compound as a pale yellow solid;

1H NMR (400 MHz, d6-DMSO) δ 9.84 (1H, s), 7.57-7.20 (7H, m), 3.09 (3H, s), 2.19 (3H, s), 2.08 (3H, s), 2.05 (1H, m), 1.05 (2H, m), 0.82 (2H, m).

LC-MS: Rt 1.25 mins (100%); m/z 481.1 [M+H]⁺; Method 2minLowpH.

### Example 003

### 4-Chloro-5-(2-chloro-4-(difluoromethoxy)phenyl)-N-(2-(2-fluorophenyl)-1,5-dimethyl-3-oxo-2,3-dihydro-1H-pyrazol-4-yl)isoxazole-3-carboxamide

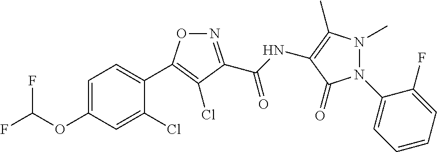 [SMILES: Cc1c(NC(=O)c2noc(-c3ccc(OC(F)F)cc3Cl)c2Cl)c(=O)n(-c2ccccc2F)n1C]

DMF (0.036 ml, 0.463 mmol) in DCM (4 ml) was treated with oxalyl chloride (0.024 ml, 0.278 mmol) at 0° C. To this solution, 4-chloro-5-(2-chloro-4-(difluoromethoxy)phenyl)isoxazole-3-carboxylic acid (60 mg, 0.185 mmol) was added. After 10 min, 4-amino-2-(2-fluorophenyl)-1,5-dimethyl-1H-pyrazol-3(2H)one (commercially available) (41 mg, 0.185 mmol) and TEA (0.103 ml, 0.741 mmol) were added. After 5 min at 0-5° C., the mixture evaporated to dryness. Purification by preparative HPLC (Gilson): Column: Waters SunFire C18, 5 um, 30×100 mm, solventA: Water+0.1% TFA, solventB: acetonitrile Gradient: 5-100% ACN in 20 min, 30 ml/min. The product fraction was evaporated with diethyl ether to give a white foam.

LC-MS: Rt 2.11 min; m/z 526[M+H]⁺; Method A, ¹H-NMR (400 MHz, CDCl₃) δ 8.39 (s, br, 1H) 7.57 (d, 1H) 7.26-7.52 (m, 5H) 7.20 (m, 1H) 7.20 (dd, 1H) 6.61 (t, 1H, J=72.24 Hz) 3.23 (s, 3H) 2.37 (s, 3H).

The compounds of the following tabulated examples (Table 1) were prepared by a similar method to that of Example 003 from the appropriate isoxazole-3-carboxylic acid and amino-pyrazolone (preparation described hereinafter).

TABLE 1

| Ex. | Structure | Name | Analytical data |
|---|---|---|---|
| 004 | N-(2-(2- Fluorophenyl)-1,5- dimethyl-3-oxo-2,3- dihydro-1H- pyrazol-4-yl)-5-(4- methoxyphenyl)-4- methylisoxazole-3- carboxamide | 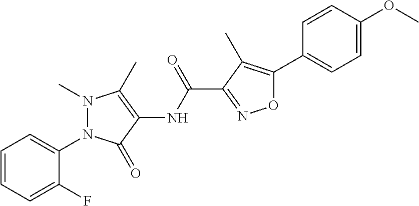 [SMILES: COc1ccc(-c2onc(C(=O)Nc3c(C)n(C)n(-c4ccccc4F)c3=O)c2C)cc1] | LC-MS: Rt 1.93 min; m/z 437 [M + H]⁺; Method A, ¹H- NMR (400 MHz, CDCl3) δ 8.14 (s,br, 1 H) 7.66-7.69 (m, 2 H) 7.37-7.45 (m, 2 H) 7.23-7.29 (m, 2H) 7.01- 7.04(m, 2H) 3.88(s, 3H) 3.14(s, 3H) 2.45(s, 3H) 2.34(s, 3H) |
| 005 | 5-(2-Fluoro-4- methoxyphenyl)-N- (2-(2-fluorophenyl)- 1,5-dimethyl-3-oxo- 2,3-dihydro-1H- pyrazol-4-yl)-4- methylisoxazole-3- carboxamide | 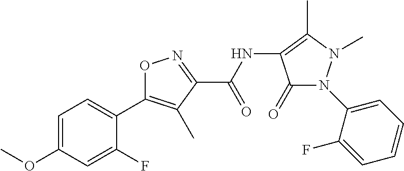 [SMILES: COc1ccc(-c2onc(C(=O)Nc3c(C)n(C)n(-c4ccccc4F)c3=O)c2C)c(F)c1] | LC-MS: Rt 1.97 min; m/z 455 [M + H]⁺; Method A, ¹H- NMR (400 MHz, CDCl3) δ 8.31 (s, 1H)7.38-7.51 (m, 3H) 7.26-7.32 (m, 2H) 6.84(dd, 1H) 6.75 (dd, 1H) 3.87 (s 3H ) 3.22 (s 3H) 2.35 (s, 3H) 2.27 (d, 3H) |
| 006 | 5-(2-Chloro-4- methoxyphenyl)-N- (2-(2-fluorophenyl)- 1,5-dimethyl-3-oxo- 2,3-dihydro-1H- pyrazol-4-yl)-4- methylisoxazole-3- carboxamide | 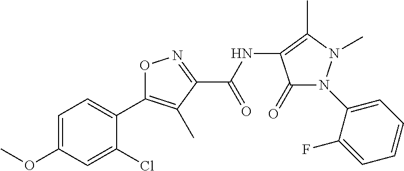 [SMILES: COc1ccc(-c2onc(C(=O)Nc3c(C)n(C)n(-c4ccccc4F)c3=O)c2C)c(Cl)c1] | LC-MS: Rt 2.03 min; m/z 471 [M + H]⁺; Method A, ¹H- NMR (400 MHz, CDCl3) δ 8.19 (s, 1H) 7.32-7.47 (m, 3H) 7.23-7.29 (m, 2H) 7.06 (d, 1H) 6.2 (dd, 1H ) 3.87(s 3H) 3.16 (s 3H) 2.34 (s, 3H) 2.21 (d, 3H) |
| 007 | N-(2-(2- Fluorophenyl)-1,5- dimethyl-3-oxo-2,3- dihydro-1H- pyrazol-4-yl)-4- methyl-5-(4- (trifluoromethoxy) phenyl)isoxazole-3- carboxamide | 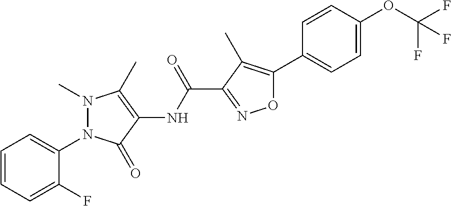 [SMILES: Cc1c(C(=O)Nc2c(C)n(C)n(-c3ccccc3F)c2=O)noc1-c1ccc(OC(F)(F)F)cc1] | LC-MS: Rt 2.16 min; m/z 491 [M + H]⁺; Method A, ¹H- NMR (400 MHz, CDCl3) δ 8.25 (s, 1H) 7.76(d, 2H) 7.25-7.72 (m, 6H) 3.17 (s, 3H) 2.47 (s, 3H) 2.34 (s, 3H) |
| 008 | N-(2-(2- Fluorophenyl)-1,5- dimethyl-3-oxo-2,3- dihydro-1H- pyrazol-4-yl)-4- methyl-5-(4- (trifluoromethyl)phenyl) isoxazole-3- carboxamide | 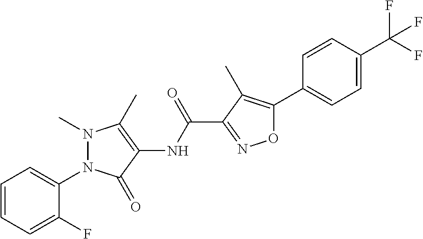 [SMILES: Cc1c(C(=O)Nc2c(C)n(C)n(-c3ccccc3F)c2=O)noc1-c1ccc(C(F)(F)F)cc1] | LC-MS: Rt 2.13 min; m/z 475 [M + H]⁺; Method A, ¹H- NMR (400 MHz , CDCl3) δ 8.75 (s, 1H) 7.77-7.88 (m, 4H) 7.23-7.53 (m, 4H) 3.26 (s, 3H) 2.50 (s, 3H) 2.38 (s, 3H) |
| 009 | 5-(2-Chloro-4- (trifluoromethoxy) phenyl)-N-(2-(2- fluorophenyl)-1,5- dimethyl-3-oxo-2,3- dihydro-1H- pyrazol-4-yl)-4- methylisoxazole-3- carboxamide | 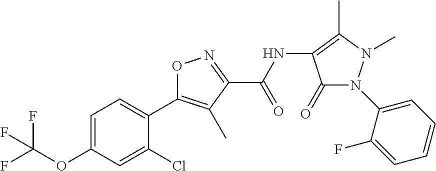 [SMILES: Cc1c(C(=O)Nc2c(C)n(C)n(-c3ccccc3F)c2=O)noc1-c1ccc(OC(F)(F)F)cc1Cl] | LC-MS: Rt 2.22 min; m/z 525 [M + H]⁺; Method A, ¹H- NMR (400 MHz, CDCl3) δ 8.52 (s, 1H) 7.39-7.52 (m, 4H) 7.25-7.33 (m, 3H) 3.24 (s, 3H) 2.37 (s, 3H) 2.24(s, 3H) |
| 010 | 5-(2-Chloro-4- (trifluoromethoxy) phenyl)-N-(2- cyclohexyl-1,5- dimethyl-3-oxo-2,3- dihydro-1H- pyrazol-4-yl)-4- methylisoxazole-3- carboxamide | 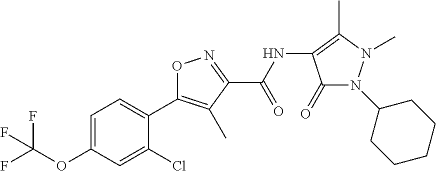 [SMILES: Cc1c(C(=O)Nc2c(C)n(C)n(C3CCCCC3)c2=O)noc1-c1ccc(OC(F)(F)F)cc1Cl] | LC-MS: Rt 2.26 min; m/z 513 [M + H]⁺; Method A, ¹H- NMR (400 MHz, CDCl3) δ 8.72 (s, br,1H) 7.47(d, 1H) 7.41 (s, 1H) 7.27 (d, 1H) 4.14-4.21 (m, 1H) 3.54(s, 3H) 2.26 (s 3H) 2.09-2.17 (m, 1H) 1.87-1.93 (m, 4H) 1.70-1.73(m, 1H) 1.10- 1.42(m, 3H) |
| 011 | N-(2-(2- Fluorophenyl)-1,5- dimethyl-3-oxo-2,3- dihydro-1H- pyrazol-4-yl)-4- methyl-5-(4- (methylthio)phenyl) isoxazole-3- carboxamide | 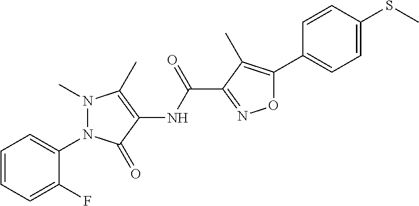 [SMILES: CSc1ccc(-c2onc(C(=O)Nc3c(C)n(C)n(-c4ccccc4F)c3=O)c2C)cc1] | LC-MS: Rt 2.05 min; m/z 453 [M + H]⁺; Method A, ¹H- NMR (400 MHz, CDCl3) δ 8.36(s, br,1H) 7.63 (d, 2H) 7.39-7.58 (m, 2H) 7.25 7.35 (M, 4H) 3.20(s, 3H) 2.53 (s, 3H) 2.45(s, 3H) 2.35(s, 3H) |
| 012 | 5-(4- (Dimethylamino) phenyl)-N-(2-(2- fluorophenyl)-1,5- dimethyl-3-oxo-2,3- dihydro-1H- pyrazol-4-yl)-4- methylisoxazole-3- carboxamide | 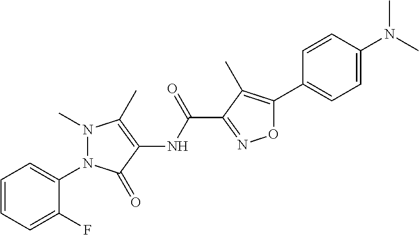 [SMILES: Cc1c(C(=O)Nc2c(C)n(C)n(-c3ccccc3F)c2=O)noc1-c1ccc(N(C)C)cc1] | LC-MS: Rt 1.75 min; m/z 450 [M + H]⁺; Method A, ¹H- NMR (400 MHz , CDCl3) δ 8.44(s, br, 1H) 7.69(d, 2H) 7.41-7.55(m, 2H) 7.29- 7.34(m, 2H) 7.08(d, 2H) 3.30(s, 3H) 3.10(s, 3H) 2.44(s, 3H) 2.36(s, 3H) |
| 013 | N-(2-(2- Fluorophenyl)-1,5- dimethyl-3-oxo-2,3- dihydro-1H- pyrazol-4-yl)-5-(4- methoxy-2-(trifluoro methyl)phenyl)-4- methylisoxazole-3- carboxamide | 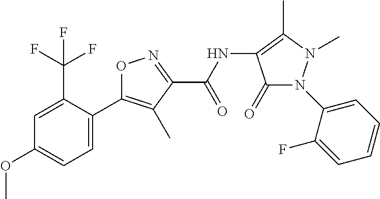 [SMILES: COc1ccc(-c2onc(C(=O)Nc3c(C)n(C)n(-c4ccccc4F)c3=O)c2C)c(C(F)(F)F)c1] | LC-MS: Rt 2.11 min; m/z 505 [M + H]⁺; Method A, ¹H- NMR (400 MHz , CDCl3) δ 8.54(d 1H) 8.31(s, br, 1H) 7.39(dd, 1H) 7.39-7.50(m, 2H) 7.26-7.32(m, 2H) 6.89(d, 1H) 4.00(s, 3H) 3.22(s, 3H) 2.44(s, 3H) 2.35(s, 3H) |
| 014 | N-(2-Cyclohexyl- 1,5-dimethyl-3-oxo- 2,3-dihydro-1H- pyrazol-4-yl)-5- (2,4-dichloro phenyl)-4-methyl isoxazole-3- carboxamide | 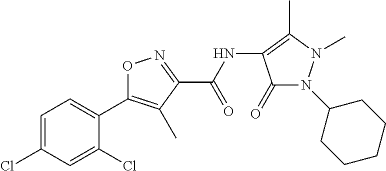 [SMILES: Cc1c(C(=O)Nc2c(C)n(C)n(C3CCCCC3)c2=O)noc1-c1ccc(Cl)cc1Cl] | LC-MS: Rt 2.17 min; m/z 463 [M + H]⁺; Method A, ¹H- NMR (400 MHz , CDCl3) δ 8.53(s, br, 1H) 7.56(m,1H) 7.38(d, 2H) 4.10- 4.17(m,1H) 3.46(s, 3H) 2.25(s, 3H) 2.19(s, 3H) 2.05-2.15(m, 2H) 1.85- 1.93(m, 4H) 1.71(d, 1H) 1.21-1.40(m, 3H) |
| 015 | 5-(2-Chloro-4- methoxyphenyl)-N- (2-cyclohexyl-1,5- dimethyl-3-oxo-2,3- dihydro-1H- pyrazol-4-yl)-4- methylisoxazole-3- carboxamide | 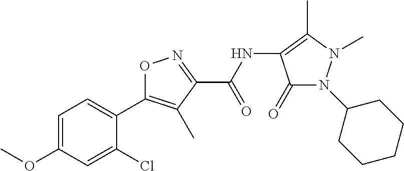 [SMILES: COc1ccc(-c2onc(C(=O)Nc3c(C)n(C)n(C4CCCCC4)c3=O)c2C)c(Cl)c1] | LC-MS: Rt 2.08 min; m/z 459 [M + H]⁺; Method A, ¹H- NMR (400 MHz , CDCl3) δ 8.37(s, br, 1H) 7.33(d, 1H) 7.05(d, 1H) 6.92(dd, 1H) 4.07-4.17(m, 1H) 3.86(s, 3H) 3.38(s, 3H) 2.23(s, 3H) 2.18(s, 3H) 2.01- 2.10(m, 2H) 1.84-1.91(m, 4H) 1.67-1.72(m, 1H)1.20- 1.41(m, 3H) |
| 016 | N-(2-Cyclohexyl- 1,5-dimethyl-3-oxo- 2,3-dihydro-1H- pyrazol-4-yl)-5-(4- methoxy-2- (trifluoromethyl)phenyl methylisoxazole-3- carboxamide | 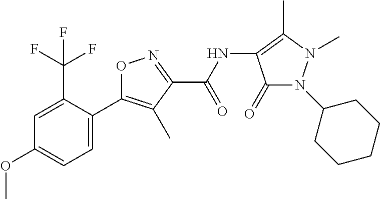 [SMILES: COc1ccc(-c2onc(C(=O)Nc3c(C)n(C)n(C4CCCCC4)c3=O)c2C)c(C(F)(F)F)c1] | LC-MS: Rt 2.14 min; m/z 492 [M + H]⁺; Method A, ¹H- NMR (400 MHz , CDCl3) δ 8.64(s, br,1H9 7.32- 7.35(m, 2H) 7.15(dd, 1H) 4.09-4.17(m, 1H) 3.92(s, 3H) 3.45(s, 3H) 2.26(s, 3H) 2.05-2.12(m, 2H) 1.84-1.93(m, 4H) 1.07(d, 1H) 1.23-1.41(m, 3H) |
| 017 | N-(2-Cyclohexyl- 1,5-dimethyl-3-oxo- 2,3-dihydro-1H- pyrazol-4-yl)-5-(4- cyclopropyl-2- fluorophenyl)-4- methylisoxazole-3- carboxamide | 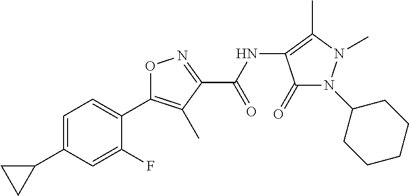 [SMILES: Cc1c(C(=O)Nc2c(C)n(C)n(C3CCCCC3)c2=O)noc1-c1ccc(C2CC2)cc1F] | LC-MS: Rt 2.16 min; m/z 453 [M + H]⁺; Method A, ¹H- NMR (400 MHz, CDCl3) δ 8.41(s, br, 1H) 7.41(t, 1H) 6.98(d, 1H) 6.86(d, 1H) 4.07-4.15(m, 1H) 3.39(s, 3H) 2.24(s, 3H) 2.03- 2.11(m, 2H) 1.83-1.96(m, 5H) 1.70(d, 1H) 1.22- 1.41(m, 3H) 1.07(m, 2H) 0.77(m, 2H) |
| 018 | 5-(4-Cyclopropyl-2- fluorophenyl)-N-(2- (2-fluorophenyl)- 1,5-dimethyl-3-oxo- 2,3-dihydro-1H- pyrazol-4-yl)-4- methylisoxazole-3- carboxamide | 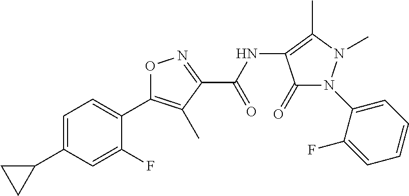 [SMILES: Cc1c(C(=O)Nc2c(C)n(C)n(-c3ccccc3F)c2=O)noc1-c1ccc(C2CC2)cc1F] | LC-MS: Rt 2.13 min; m/z 465 [M + H]⁺; Method A, ¹H- NMR (400 MHz, CDCl3) δ 8.40(s, br, 1H) 7.37- 7.49(m, 3H)7.24- 7.31(m,2H) 6.94-6.98(m, 1H9 6.83-6.88(m, 1H) 3.22(s, 3H) 2.34(s, 3H) 2.28(s, 3H) 1.91-1.97(m, 1H) 1.05-1.09(m, 2H) 0.75-0.79(m, 2H) |
| 019 | 5-(2-Chloro-4- cyclopropylphenyl)- N-(2-cyclohexyl- 1,5-dimethyl-3-oxo- 2,3-dihydro-1H- pyrazol-4-yl)-4- methylisoxazole-3- carboxamide | 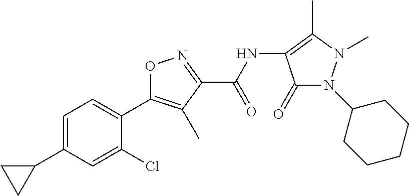 [SMILES: Cc1c(C(=O)Nc2c(C)n(C)n(C3CCCCC3)c2=O)noc1-c1ccc(C2CC2)cc1Cl] | LC-MS: Rt 2.21 min; m/z 469 [M + H]⁺; Method A, ¹H- NMR (400 MHz, CDCl3) δ 8.57(s, br, 1H) 7.29(s, 1H) 7.20(d, 1H) 7.06(dd, 1H) 4.09-4.16(m, 1H) 3.43(s, 3H) 2.25(s, 3H) 2.18(s, 3H) 2.04-2.11(m, 2H) 1.85-1.94(m, 5H) 1.71(d, 1H) 1.21-1.40(M, 3H) 1.05-1.09(m, 2H) 0.76- 0.79(m, 2H) |
| 020 | 4-Chloro-5-(2,4- dichlorophenyl)-N- (2-(2-fluorophenyl)- 1,5-dimethyl-3-oxo- 2,3-dihydro-1H- pyrazol-4- yl)isoxazole-3- carboxamide | 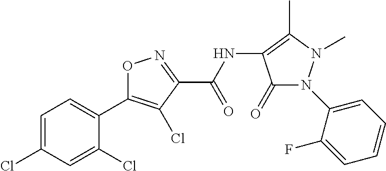 [SMILES: Cc1c(NC(=O)c2noc(-c3ccc(Cl)cc3Cl)c2Cl)c(=O)n(-c2ccccc2F)n1C] | LC-MS: Rt 2.15 min; m/z 496 [M + H]⁺; Method A, ¹H- NMR (400 MHz, CDCl3) δ 8.75(s, br, 1H) 7.59(d, 1H) 7.48-7.53(m, 2H) 7.40- 7.44(M, 2H) 7.27-7.34(m, 2H) 3.25(s, 3H) 2.40(s, 3H) |
| 021 | 4-Chloro-5-(3- chloro-2-fluoro-4- methoxy phenyl)- N-(2-(2- fluorophenyl)-1,5- dimethyl-3-oxo-2,3- dihydro-1H- pyrazol-4- yl)isoxazole-3- carboxamide | 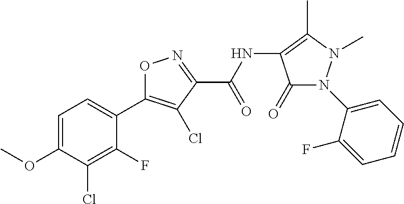 [SMILES: COc1ccc(-c2onc(C(=O)Nc3c(C)n(C)n(-c4ccccc4F)c3=O)c2Cl)c(F)c1Cl] | LC-MS: Rt 2.06 min; m/z 509 [M + H]⁺; Method A, ¹H- NMR (400 MHz, CDCl3) δ 8.12(s, br, 1H) 7.76(d, 1H) 7.36-7.44(m, 2H) 7.23- 7.30(m, 2H) 6.83(d, 1H) 3.98(s, 3H) 3.15(s, 3H) 2.35(s, 3H) |
| 022 | 4-Chloro-5-(2- fluoro-4- methoxyphenyl)-N- (2-(2-fluorophenyl)- 1,5-dimethyl-3-oxo- 2,3-dihydro-1H- pyrazol-4-yl) isoxazole-3- carboxamide | 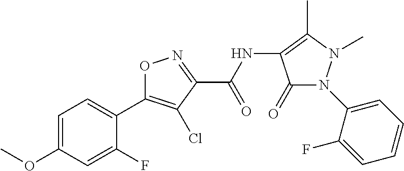 [SMILES: COc1ccc(-c2onc(C(=O)Nc3c(C)n(C)n(-c4ccccc4F)c3=O)c2Cl)c(F)c1] | LC-MS: Rt 1.85 min; m/z 475.1 [M + H]⁺; Method A, ¹H-NMR (400 MHz, CDCl3) δ 7.67 (triplet, 1H), 7.49-7.35 (m, 2H), 7.31-7.30 (m, 2H), 7.21 (s, 1H), 6.86-6.76 (m, 2H), 3.83 (s, 3H), 3.20 (s, 3H), 2.38 (s, 3H) |
| 023 | 4-Chloro-N-(2- cyclohexyl-1,5- dimethyl-3-oxo-2,3- dihydro-1H- pyrazol-4-yl)-5- (2,4- dichlorophenyl) isoxazole-3- carboxamide | 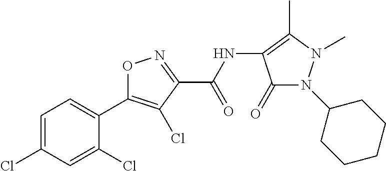 [SMILES: Cc1c(NC(=O)c2noc(-c3ccc(Cl)cc3Cl)c2Cl)c(=O)n(C2CCCCC2)n1C] | LC-MS: Rt 2.18 min; m/z 485 [M + H]⁺; Method A, ¹H- NMR (400 MHz, CDCl3) δ 8.40(s, br, 1H) 7.58(d, 1H) 7.48(d, 1H) 7.41(dd, 1H) 4.04-4.11(m, 1H) 3.34(s, 3H) 2.25(s, 3H) 1.99- 2.08(m, 2H) 1.84-1.91(m, 4H) 1.70, (d, 1H) 1.18- 1.42(m, 3H) |
| 026 | 4-chloro-5-(2- chloro-4- (trifluoromethoxy) phenyl)-N-(2- cyclohexyl-1,5- dimethyl-3-oxo-2,3- dihydro-1H- pyrazol-4- yl)isoxazole-3- carboxamide | 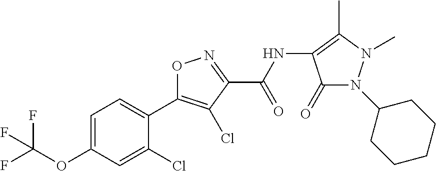 [SMILES: Cc1c(NC(=O)c2noc(-c3ccc(OC(F)(F)F)cc3Cl)c2Cl)c(=O)n(C2CCCCC2)n1C] | LC-MS: Rt 2.27 min; m/z 532 [M + H]⁺; Method A, ¹H- NMR (400 MHz, CDCl3) δ 8.46(s, br 1H) 7.60(d, 1H) 7.44(s, 1H) 7.29(d 1H) 4.06-4.12 (m, 1H) 3.37(s, 3H) 2.26(s, 3H) 2.02- 2.10(m, 2H) 1.84-1.92(m, 4H) 1.71(d, 1H) 1.21- 1.41(m, 3H) |
| 027 | 4-Chloro-5-(2- chloro-4- methoxyphenyl)-N- (2-(2-fluorophenyl)- 1,5-dimethyl-3-oxo- 2,3-dihydro-1H- pyrazol-4- yl)isoxazole-3- carboxamide | 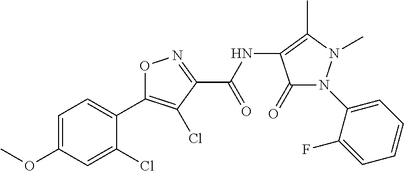 [SMILES: COc1ccc(-c2onc(C(=O)Nc3c(C)n(C)n(-c4ccccc4F)c3=O)c2Cl)c(Cl)c1] | LC-MS: Rt 2.02 min; m/z 491 [M + H]⁺; Method A, ¹H- NMR (400 MHz, DMSO- d₆) δ 10.13 (s, 1 H) 7.65 (d, 1 H) 7.56-7.50 (m, 1 H) 7.46-7.32 (m, 4 H) 7.16- 7.13 (m, 1 H) 3.87 (s, 3 H) 3.10 (s, 3 H) 2.19 (s, 3 H) |
| 028 | 4-Chloro-5-(2- chloro-4- methoxyphenyl)-N- (2-cyclohexyl-1,5- dimethyl-3-oxo-2,3- dihydro-1H- pyrazol-4- yl)isoxazole-3- carboxamide | 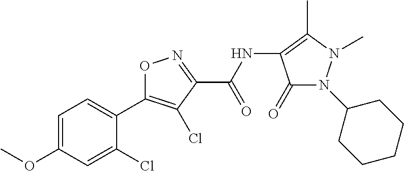 [SMILES: COc1ccc(-c2onc(C(=O)Nc3c(C)n(C)n(C4CCCCC4)c3=O)c2Cl)c(Cl)c1] | LC-MS: Rt 2.09 min; m/z 479 [M + H]⁺; Method A, ¹H- NMR (400 MHz, DMSO- d₆) δ 9.94 (broad s, 1H) 7.63 (d, 1H) 7.31 (d, 1H) 7.15-7.13 (m, 1H) 3.97- 3.89 (broad m, 1H) 3.87 (s, 3H) 3.24 (s, 3H) 2.07- 1.95 (broad m, 5H) 1.79- 1.60 (broad m, 5H) 1.36- 1.13 (broad m, 3H) |
| 029 | 4-Chloro-5-(2- chloro-4- (difluoromethoxy) phenyl)-N-(2- cyclohexyl-1,5- dimethyl-3-oxo-2,3- dihydro-1H- pyrazol-4- yl)isoxazole-3- carboxamide | 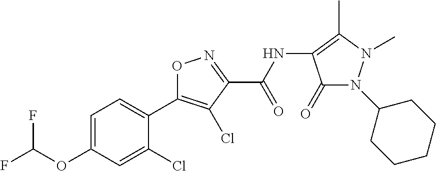 [SMILES: Cc1c(NC(=O)c2noc(-c3ccc(OC(F)F)cc3Cl)c2Cl)c(=O)n(C2CCCCC2)n1C] | LC-MS: Rt 2.14 min; m/z 515 [M + H]⁺; Method A, ¹H- NMR (400 MHz, CDCl3) δ 7.56(d1H) 7.34(d, 1H) 7.19(dd, 1H) 6.60(t, 1H, J = 72.63 Hz) 4.11-4,17(m, 1H) 3.50(s, 3H) 2.29(s, 3H) 2.09-2.17(m, 2H) 1.86- 1.94(m, 4H) 1.72(d, 1H) 1.24-1.40(m, 3H) |
| 030 | 4-Chloro-5-(2- chloro-4- cyclopropylphenyl)- N-(2-(2- fluorophenyl)-1,5- dimethyl-3-oxo-2,3- dihydro-1H- pyrazol-4- yl)isoxazole-3- carboxamide | 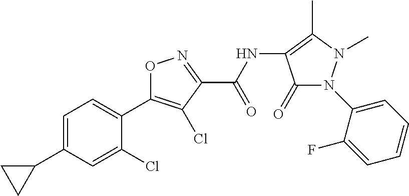 [SMILES: Cc1c(NC(=O)c2noc(-c3ccc(C4CC4)cc3Cl)c2Cl)c(=O)n(-c2ccccc2F)n1C] | LC-MS: Rt 2.18 min; m/z 501 [M + H]⁺; Method A, ¹H- NMR (400 MHz, CDCl3) δ 8.31(s, br, 1H) 7.38- 7.49(m, 3H) 7.25-7.31(m, 2H) 7.22(m, 1H) 7.09(dd, 1H) 3.20(s, 3H) 2.37(s, 3H) 1.91-1.98(m, 1H) 1.07-1.12(m,2H) 0.77-0.82 (m, 2H) |
| 031 | 4-Chloro-5-(2- chloro-4- cyclopropylphenyl)- N-(2-cyclohexyl- 1,5-dimethyl-3-oxo- 2,3-dihydro-1H- pyrazol-4- yl)isoxazole-3- carboxamide | 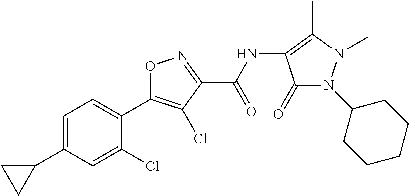 [SMILES: Cc1c(NC(=O)c2noc(-c3ccc(C4CC4)cc3Cl)c2Cl)c(=O)n(C2CCCCC2)n1C] | LC-MS: Rt 2.21 min; m/z 489 [M + H]⁺; Method A, ¹H- NMR (400 MHz, CDCl3) δ 8.49(s, br, 1H) 7.40(d, 1H) 7.21(d, 1H) 7.08(dd, 1H) 4.09-4.13(M, 1H) 3.37(s, 3H) 2.22(s, 3H) 2.01- 2.11(m, 2H) 1.83-1.95(m, 5H) 1.69(d, 1H) 1.21- 1.41(m, 3H) 1.06-1.11(m, 2H) 0.77-0.80(m, 2H) |
| 032 | 5-(2-Chloro-4- (trifluoromethoxy) phenyl)-N-(2- cyclohexyl-1,5- dimethyl-3-oxo-2,3- dihydro-1H- pyrazol-4- yl)isoxazole-3- carboxamide | 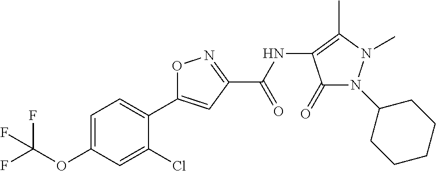 [SMILES: Cc1c(NC(=O)c2cc(-c3ccc(OC(F)(F)F)cc3Cl)on2)c(=O)n(C2CCCCC2)n1C] | LC-MS: Rt 2.18 min; m/z 499 [M + H]⁺; Method A, ¹H- NMR (400 MHz , CDCl3) δ 8.87(s, br, 1H) 8.00(d, 1H) 7.38-7.42(m, 2H) 7.28(d, 1H) 4.10-4.18(M, 1H) 3.49(s, 3H) 2.25(s, 3H) 2.07-2.17(m, 2H) 1.85- 1.93(m, 4H) 1.71(d, 1H) 1.21-1.41(m, 3H) |
| 033 | 5-(2-Chloro-4- methoxyphenyl)-4- fluoro-N-(2-(2- fluorophenyl)-1,5- dimethyl-3-oxo-2,3- dihydro-1H- pyrazol-4- yl)isoxazole-3- carboxamide | 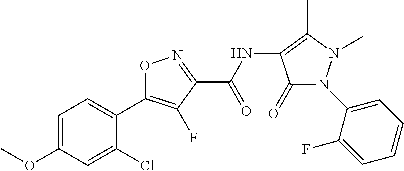 [SMILES: COc1ccc(-c2onc(C(=O)Nc3c(C)n(C)n(-c4ccccc4F)c3=O)c2F)c(Cl)c1] | LC-MS: Rt 1.97 min; m/z 475.0 [M + H]⁺; Method A, ¹H-NMR (400 MHz, DMSO-d₆) δ 9.93 (s, 1 H) 7.88 (d, 1 H) 7.53-7.34 (m, 6 H) 3.95 (s, 3 H) 3.09 (s, 3 H) 2.18 (s, 3 H) |
| 034 | 4-Bromo-5-(2- chloro-4- methoxyphenyl)-N- (2-cyclohexyl-1,5- dimethyl-3-oxo-2,3- dihydro-1H- pyrazol-4- yl)isoxazole-3- carboxamide | 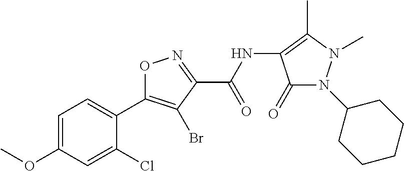 | LC-MS: Rt 2.09 min; m/z 479 [M + H]⁺; Method A, ¹H- NMR (400 MHz, DMSO- d₆) δ 9.92 (s, 1H) 7.61- 7.59 (m, 1H) 7.31-7.30 (m, 1H) 7.14-7.11 (m, 1H) 3.98-3.90 (broad m, 1H) 3.86 (s, 3H) 3.22 (s, 3H) 2.05-1.95 (broad m, 5H) 1.79-1.59 (broad m, 5H) 1.36-1.13 (broad m, 3H) |
| 035 | 4-Bromo-5-(2- chloro-4- methoxyphenyl)-N- (2-(2-fluorophenyl)- 1,5-dimethyl-3-oxo- 2,3-dihyd 2,3-1H- pyrazol-4- yl)isoxazole-3- carboxamide | 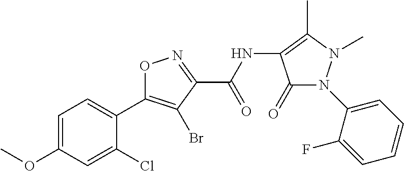 [SMILES: COc1ccc(-c2onc(C(=O)Nc3c(C)n(C)n(-c4ccccc4F)c3=O)c2Br)c(Cl)c1] | LC-MS: Rt 2.05 min; m/z 536.9 [M + H]⁺; Method A, ¹H-NMR (400 MHz, DMSO-d₆) δ 10.10 (s, 1 H) 7.61 (d, 1 H) 7.55-7.50 (m, 1 H) 7.46-7.30 (m, 4 H) 7.15-7.12 (m, 1 H) 3.86 (s, 3 H) 3.09 (s, 3 H) 2.19 (s, 3 H) |
| 036 | 4-Bromo-N-(2- cyclohexyl-1,5- dimethyl-3-oxo-2,3- dihydro-1H- pyrazol-4-yl)-5- (2,4- dichlorophenyl) isoxazole-3- carboxamide | 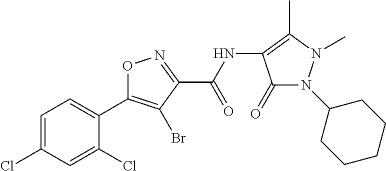 [SMILES: Cc1c(NC(=O)c2noc(-c3ccc(Cl)cc3Cl)c2Br)c(=O)n(C2CCCCC2)n1C] | LC-MS: Rt 2.20 min; m/z 528.9 [M + H]⁺; Method A, ¹H-NMR (400 MHz, DMSO-d₆) δ 9.99 (s, 1H) 7.98 (m, 1H) 7.77 (d, 1H) 7.72-7.69 (m, 1H) 4.01- 3.93 (broad m, 1H) 3.17 (s, 3H) 2.08-1.97 (broad m, 5H) 1.81-1.61 (broad m, 5H) 1.38-1.11 (broad m, 3H) |
| 037 | 4-Bromo-5-(2,4- dichlorophenyl)-N- (2-(2-fluorophenyl)- 1,5-dimethyl-3-oxo- 2,3-dihydro-1H- pyrazol-4- yl)isoxazole-3- carboxamide | 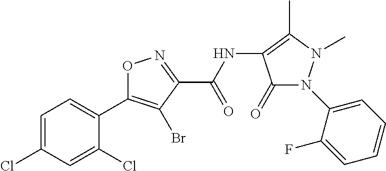 [SMILES: Cc1c(NC(=O)c2noc(-c3ccc(Cl)cc3Cl)c2Br)c(=O)n(-c2ccccc2F)n1C] | LC-MS: Rt 2.16 min; m/z 540.9 [M + H]⁺; Method A, ¹H-NMR (400 MHz, DMSO-d₆) δ 10.15 (s, 1 H) 7.97 (m, 1 H) 7.76 (d, 1 H) 7.70-7.63 (m, 1 H) 7.55-7.50 (m, 1 H) 7.46- 7.34 (m, 3H) 3.09 (s, 3 H) 2.19 (s, 3 H) |
| 038 | 5-(2-Chloro-4- methoxyphenyl)-N- (2-cyclohexyl-1,5- dimethyl-3-oxo-2,3- dihydro-1H- pyrazol-4-yl)-4- cyclopropy lisoxazole-3- carboxamide | 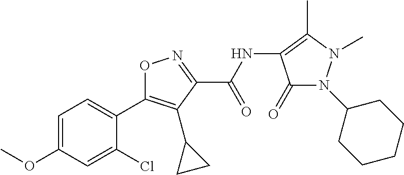 [SMILES: COc1ccc(-c2onc(C(=O)Nc3c(C)n(C)n(C4CCCCC4)c3=O)c2C2CC2)c(Cl)c1] | LC-MS: Rt 2.13 min; m/z 485.1 [M + H]⁺; Method A, ¹H-NMR (400 MHz, DMSO-d₆) δ 9.66 (s, 1H) 7.51 (d, 1H) 7.26 (d, 1H) 7.09-7.07 (m, 1H) 3.96- 3.89 (broad m, 1H) 3.85 (s, 3H) 3.23 (s, 3H) 2.05- 1.95 (broad m, 5H) 1.81- 1.73 (broad m, 3H) 1.68- 1.59 (broad m, 3H) 1.35- |
| | | | 1.13 (broad m, 3H) 0.70- |
| | | | 0.65 (m, 2H) 0.37-0.33 (m, |
| | | | 2H) |
| 039 | N-(2-Cyclohexyl- 1,5-dimethyl-3-oxo- 2,3-dihydro-1H- pyrazol-4-yl)-4- cyclopropyl-5-(2,4- dichlorophenyl) isoxazole-3- carboxamide | 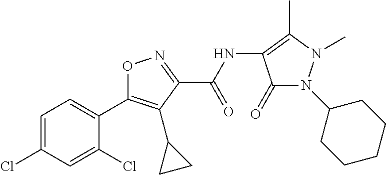 [SMILES: Cc1c(NC(=O)c2noc(-c3ccc(Cl)cc3Cl)c2C2CC2)c(=O)n(C2CCCCC2)n1C] | LC-MS: Rt 2.24 min; m/z 489.1 [M + H]⁺; Method A, ¹H-NMR (400 MHz, DMSO-d₆) δ 9.74 (s, 1H) 7.91 (m, 1H) 7.67-7.61 (m, 2H) 3.98-3.91 (broad m, 1H) 3.25 (s, 3H) 2.06-1.94 (broad m, 5H) 1.83-1.73 (broad m, 3H) 1.68-1.59 (broad m, 3H) 1.36-1.10 (broad m, 3H) 0.72-0.67 |
| | | | (m, 2H) 0.36-0.32 (m, 2H) |
| 040 | 4-Cyclopropyl-5- (2,4- dichlorophenyl)-N- (2-(2-fluorophenyl)- 1,5-dimethyl-3-oxo- 2,3-dihydro-1H- pyrazol-4- yl)isoxazole-3- carboxamide | 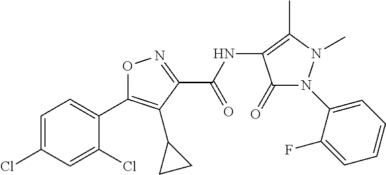 [SMILES: Cc1c(NC(=O)c2noc(-c3ccc(Cl)cc3Cl)c2C2CC2)c(=O)n(-c2ccccc2F)n1C] | LC-MS: Rt 2.21 min; m/z 501 [M + H]⁺; Method A, ¹H- NMR (400 MHz, DMSO- d₆) δ 9.92 (s, 1 H) 7.91 (d, 1 H) 7.68-7.62 (m, 2 H) 7.55-7.34 (m, 4 H) 3.07 (s, 3 H) 2.19 (s, 3 H) 1.85- 1.78 (m, 1H) 0.74-0.69 (m, 2H) 0.37-0.33 (m, 2H) |
| 041 | 4-Cyano-N-(2- cyclohexyl-1,5- dimethyl-3-oxo-2,3- dihydro-1H- pyrazol-4-yl)-5- (2,4- dichlorophenyl) isoxazole-3- carboxamide | 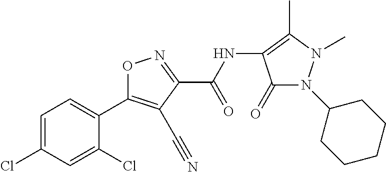 [SMILES: Cc1c(NC(=O)c2noc(-c3ccc(Cl)cc3Cl)c2C#N)c(=O)n(C2CCCCC2)n1C] | LC-MS: Rt 2.12 min; m/z 474.0 [M + H]⁺; Method A, ¹H-NMR (400 MHz, DMSO-d₆) δ 10.11 (s, 1H) 8.04 (m, 1H) 7.92 (d, 1H) 7.79-7.76 (m, 1H) 3.26 (s, 3H) 2.06 (s, 3H) 2.04-1.95 (broad m, 2H) 1.79-1.59 (broad m, 5H) 1.36-1.13 (broad m, 3H) |
| 042 | 4-Cyano-5-(2,4- dichlorophenyl)-N- (2-(2-fluorophenyl)- 1,5-dimethyl-3-oxo- 2,3-dihydro-1H- pyrazol-4- yl)isoxazole-3- carboxamide | 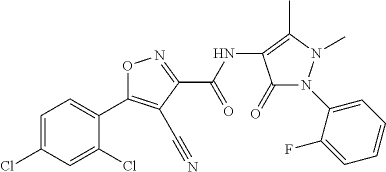 [SMILES: Cc1c(NC(=O)c2noc(-c3ccc(Cl)cc3Cl)c2C#N)c(=O)n(-c2ccccc2F)n1C] | LC-MS: Rt 2.08 min; m/z 485.9 [M + H]⁺; Method A, ¹H-NMR (400 MHz, DMSO-d₆) δ 10.30 (s, 1 H) 8.04 (m, 1 H) 7.92 (d, 1 H) 7.79-7.76 (m, 1 H) 7.55-7.50 (m, 1 H) 7.46- 7.34 (m, 3H) 3.10 (s, 3 H) 2.20 (s, 3 H) |
| 043 | N-(2-Cyclohexyl- 1,5-dimethyl-3-oxo- 2,3-dihydro-1H- pyrazol-4-yl)-5- (2,4-dichloro phenyl)-4-(trifluoro methyl)isoxazole-3- carboxamide | 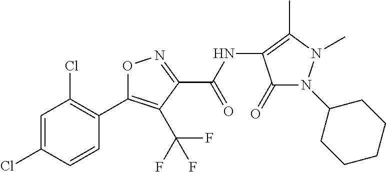 | LC-MS: Rt 2.29 min; m/z 4517.0 [M + H]⁺; Method A, ¹H-NMR (400 MHz, DMSO-d₆) δ 10.16 (s, 1H) 7.98 (d, 1H) 7.80 (d, 1H) 7.71-7.69 (m, 1H) 4.00- 3.93 (broad m, 1H) 3.28 (s, 3H) 2.04-1.95 (broad m, 5H) 1.79-1.59 (broad m, 5H) 1.36-1.12 (broad m, 3H) |
| 044 | 5-(2,4- Dichlorophenyl )-N- (2-(2-fluorophenyl)- 1,5-dimethyl-3- oxo-2,3-dihydro- 1H-pyrazol-4-yl)-4- (trifluoromethyl) isoxazole-3- carboxamide | 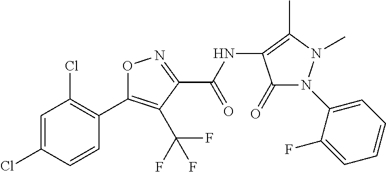 [SMILES: Cc1c(NC(=O)c2noc(-c3ccc(Cl)cc3Cl)c2C(F)(F)F)c(=O)n(-c2ccccc2F)n1C] | LC-MS: Rt 2.25 min; m/z 529.0 [M + H]⁺; Method A, ¹H-NMR (400 MHz, DMSO-d₆) δ 10.34 (s, 1 H) 7.99 (d, 1 H) 7.81 (s, 1H) 7.72-7.69 (m, 1 H) 7.56-7.50 (m, 1 H) 7.46- 7.34 (m, 3 H) 3.10 (s, 3 H) 2.17 (s, 3 H) |
| 045 | 5-(2-chloro-4- cyclopropylphenyl)- N-(2-(2,5- dimethylphenyl)- 1,5-dimethyl-3-oxo- 2,3-dihydro-1H- pyrazol-4-yl)-4- methylisoxazole-3- carboxamide | 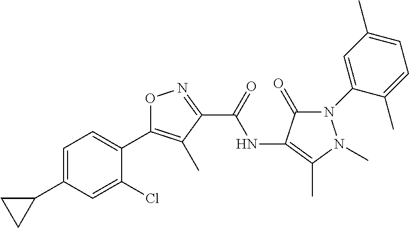 [SMILES: Cc1ccc(C)c(-n2c(=O)c(NC(=O)c3noc(-c4ccc(C5CC5)cc4Cl)c3C)c(C)n2C)c1] | (Method 2minLowpHv01); LC-MS: Rt 1.29 min m/z 491.1 [M + H]⁺ |
| 046 | 5-(2,6-difluoro-4- methoxyphenyl)-N- (2-(2-fluorophenyl)- 1,5-dimethyl-3-oxo- 2,3-dihydro-1H- pyrazol-4-yl)-4- methylisoxazole-3- carboxamide | 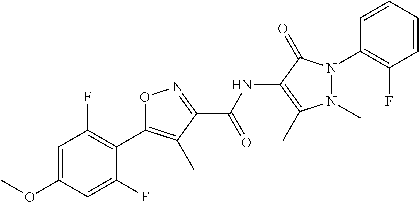 [SMILES: COc1cc(F)c(-c2onc(C(=O)Nc3c(C)n(C)n(-c4ccccc4F)c3=O)c2C)c(F)c1] | LC-MS: Rt 1.98 min; m/z 473.1 [M + H]⁺; Method A, |

### Example 47

### 5-(2-Chloro-4-(methoxymethyl)phenyl)-N-(2-cyclohexyl-1,5-dimethyl-3-oxo-2,3-dihydro-1H-pyrazol-4-yl)-4-methylisoxazole-3-carboxamide

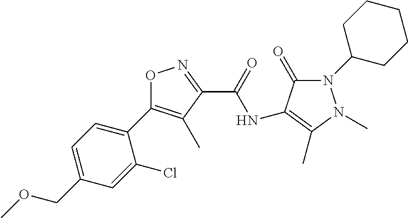 [SMILES: COCc1ccc(-c2onc(C(=O)Nc3c(C)n(C)n(C4CCCCC4)c3=O)c2C)c(Cl)c1]

To a microwave vial was added 5-bromo-N-(2-cyclohexyl-1,5-dimethyl-3-oxo-2,3-dihydro-1H-pyrazol-4-yl)-4-methylisoxazole-3-carboxamide (Intermediate L27) (149 mg, 0.375 mmol), 2-(2-chloro-4-(methoxymethyl)phenyl)-4,4,5,5-tetramethyl-1,3,2-dioxaborolane (Intermediate L29)(106 mg, 0.375 mmol), PdCl₂(dppf)-CH₂Cl₂-adduct (30.6 mg, 0.038 mmol), K₂CO₃ (156 mg, 1.125 mmol), MeCN (1.2 ml) and water (0.400 ml). The resulting mixture was heated using microwave radiation at 100° C. for 2 hr. The mixture was diluted with EtOAc and washed twice with water. The organic portion was dried over MgSO₄, filtered and concentrated under reduced pressure. Purification by preparative HPLC afforded the title compound;

LC-MS: Rt 4.88 mins; MS m/z 473.5 [M+H]⁺; Method 10minLowpH 1H NMR (400 MHz, CDCl3) δ 8.07 (1H, s), 7.54 (1H, s), 7.42 (1H, d), 7.36 (1H, d), 4.52 (2H, s), 4.14-4.04 (1H, m), 3.46 (3H, s), 3.26 (3H, s), 2.24 (3H, s), 2.24 (3H, s), 2.06-1.95 (2H, m), 1.87 (4H, br d), 1.71 (1H, br d), 1.43-1.33 (2H, m), 1.27-1.18 (1H, m).

The compounds of the following tabulated examples (Table 2) were prepared analogously to Example 047 from 5-bromo-N-(2-cyclohexyl-1,5-dimethyl-3-oxo-2,3-dihydro-1H-pyrazol-4-yl)-4-methylisoxazole-3-carboxamide (Intermediate L27) or 5-bromo-N-(2-(2-fluorophenyl)-1,5-dimethyl-3-oxo-2,3-dihydro-1H-pyrazol-4-yl)-4-methylisoxazole-3-carboxamide (Intermediate L28) and the appropriate boronic acid.

| Ex. | Structure | Name | Analytical data |
|---|---|---|---|
| 048 | 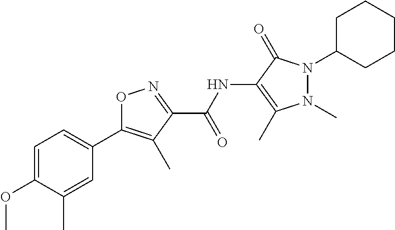 [SMILES: COc1ccc(-c2onc(C(=O)Nc3c(C)n(C)n(C4CCCCC4)c3=O)c2C)cc1C] | N-(2-Cyclohexyl-1,5- dimethyl-3-oxo-2,3- dihydro-1H-pyrazol-4- yl)-5-(4-methoxy-3- methylphenyl)-4- methylisoxazole-3- carboxamide | LC-MS: Rt 1.16 min; m/z 439.5 [MH]⁺; Method 2minHighpH |
| 049 | 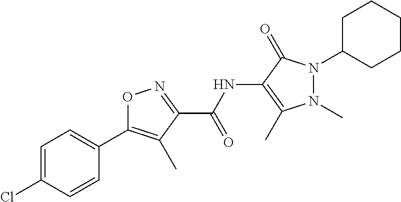 [SMILES: Cc1c(C(=O)Nc2c(C)n(C)n(C3CCCCC3)c2=O)noc1-c1ccc(Cl)cc1] | 5-(4-Chlorophenyl)-N- (2-cyclohexyl-1,5- dimethyl-3-oxo-2,3- dihydro-1H-pyrazol-4- yl)-4-methylisoxazole- 3-carboxamide | LC-MS: Rt 1.16 min; m/z 429.4 [MH]⁺; Method 2minHighpH. |
| 050 | 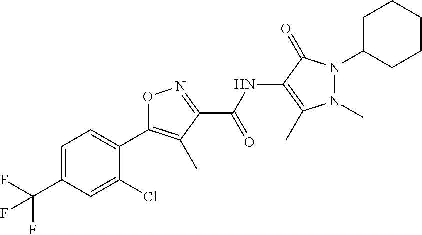 [SMILES: Cc1c(C(=O)Nc2c(C)n(C)n(C3CCCCC3)c2=O)noc1-c1ccc(C(F)(F)F)cc1Cl] | 5-(2-Chloro-4- (trifluoromethyl)phenyl)- N-(2-cyclohexyl-1,5- dimethyl-3-oxo-2,3- dihydro-1H-pyrazol-4- yl)-4-methylisoxazole- 3-carboxamide | LC-MS: Rt 1.15 min; m/z 497.5 [MH]⁺; Method 2minLow pH. |
| 051 | 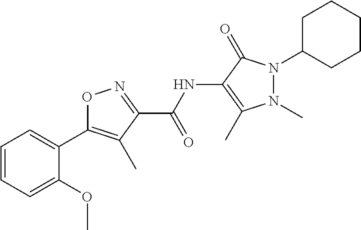 [SMILES: COc1ccccc1-c1onc(C(=O)Nc2c(C)n(C)n(C3CCCCC3)c2=O)c1C] | N-(2-Cyclohexyl-1,5- dimethyl-3-oxo-2,3- dihydro-1H-pyrazol-4- yl)-5-(2- methoxyphenyl)-4- methylisoxazole-3- carboxamide | LC-MS: Rt 1.03 min; m/z 425.3 [MH]⁺; Method 2minHighpH. |
| 052 | 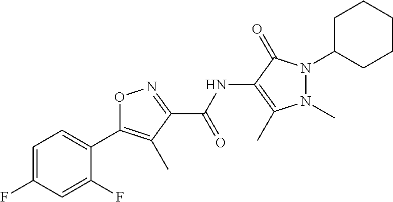 [SMILES: Cc1c(C(=O)Nc2c(C)n(C)n(C3CCCCC3)c2=O)noc1-c1ccc(F)cc1F] | N-(2-Cyclohexyl-1,5- dimethyl-3-oxo-2,3- dihydro-1H-pyrazol-4- difluorophenyl)-4- methylisoxazole-3- carboxamide | LC-MS: Rt 1.11 min; m/z 431.5[MH]⁺; Method 2minHighpH. |
| 053 | 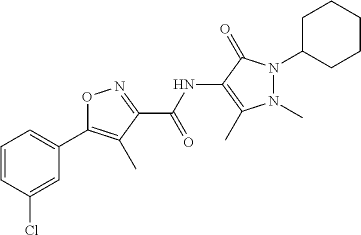 [SMILES: Cc1c(C(=O)Nc2c(C)n(C)n(C3CCCCC3)c2=O)noc1-c1cccc(Cl)c1] | 5-(3-Chlorophenyl)-N- (2-cyclohexyl-1,5- dimethyl-3-oxo-2,3- dihydro-1H-pyrazol-4- yl)-4-methylisoxazole- 3-carboxamide | LC-MS: Rt 1.17 min; m/z 429.4[MH]⁺; Method 2minHighpHv01 |
| 054 | 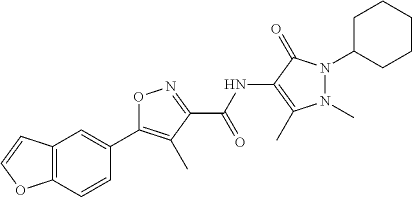 [SMILES: Cc1c(C(=O)Nc2c(C)n(C)n(C3CCCCC3)c2=O)noc1-c1ccc2occc2c1] | 5-(Benzofuran-5-yl)-N- (2-cyclohexyl-1,5- dimethyl-3-oxo-2,3- dihydro-1H-pyrazol-4- yl)-4-methylisoxazole- 3-carboxamide | LC-MS: Rt 1.13 min; m/z 434.5 [MH]⁺; Method 2minHighpH_plates |
| 055 | 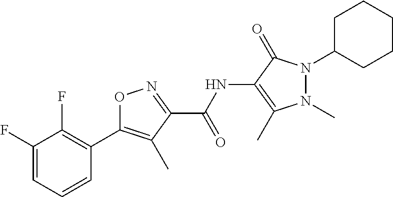 [SMILES: Cc1c(C(=O)Nc2c(C)n(C)n(C3CCCCC3)c2=O)noc1-c1cccc(F)c1F] | N-(2-Cyclohexyl-1,5- dimethyl-3-oxo-2,3- dihydro-1H-pyrazol-4- yl)-5-(2,3- difluorophenyl)-4- methylisoxazole-3- carboxamide | LC-MS: Rt 1.11 min; m/z 431.5 [MH]⁺; Method 2minHighpH. |
| 056 | 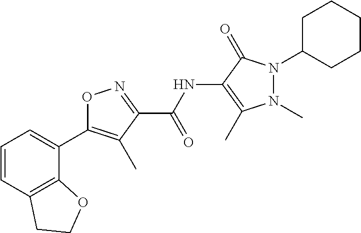 [SMILES: Cc1c(C(=O)Nc2c(C)n(C)n(C3CCCCC3)c2=O)noc1-c1cccc2c1OCC2] | N-(2-Cyclohexyl-1,5- dimethyl-3-oxo-2,3- dihydro-1H-pyrazol-4- yl)-5-(2,3- dihydrobenzofuran-7- yl)-4-methylisoxazole- 3-carboxamide | LC-MS: Rt 1.09 min; m/z 437.4 [MH]⁺; Method 2minHighpH_plates |
| 057 | 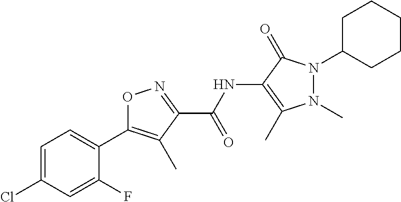 [SMILES: Cc1c(C(=O)Nc2c(C)n(C)n(C3CCCCC3)c2=O)noc1-c1ccc(Cl)cc1F] | 5-(4-Chloro-2- fluorophenyl)-N-(2- cyclohexyl-1,5- dimethyl-3-oxo-2,3- dihydro-1H-pyrazol-4- yl)-4-methylisoxazole- 3-carboxamide | LC-MS: Rt 1.17 min; m/z 447.5 [MH]⁺; Method 2minHighpH. |
| 058 | 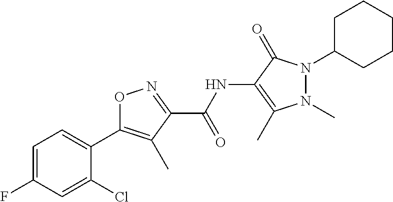 [SMILES: Cc1c(C(=O)Nc2c(C)n(C)n(C3CCCCC3)c2=O)noc1-c1ccc(F)cc1Cl] | 5-(2-Chloro-4- fluorophenyl)-N-(2- cyclohexyl-1,5- dimethyl-3-oxo-2,3- dihydro-1H-pyrazol-4- yl)-4-methylisoxazole- 3-carboxamide | LC-MS: Rt 1.14 min; m/z 447.9 [MH]⁺; Method 2minHighpH |
| 059 | 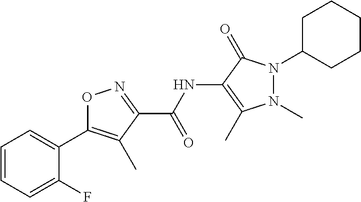 [SMILES: Cc1c(C(=O)Nc2c(C)n(C)n(C3CCCCC3)c2=O)noc1-c1ccccc1F] | N-(2-Cyclohexyl-1,5- dimethyl-3-oxo-2,3- dihydro-1H-pyrazol-4- yl)-5-(2-fluorophenyl)-4- methylisoxazole-3- carboxamide | LC-MS: Rt 1.03 min; m/z 413.5 [MH]⁺; Method 2minLowpH |
| 060 | 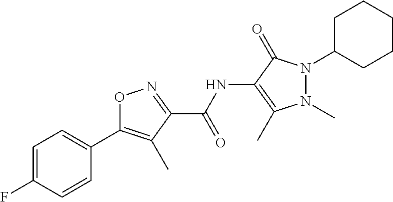 [SMILES: Cc1c(C(=O)Nc2c(C)n(C)n(C3CCCCC3)c2=O)noc1-c1ccc(F)cc1] | N-(2-Cyclohexyl-1,5- dimethyl-3-oxo-2,3- dihydro-1H-pyrazol-4- yl)-5-(4-fluorophenyl)-4- methylisoxazole-3- carboxamide | LC-MS: Rt 1.1 min; m/z 413.5 [MH]⁺; Method 2minHighpH. |
| 061 | 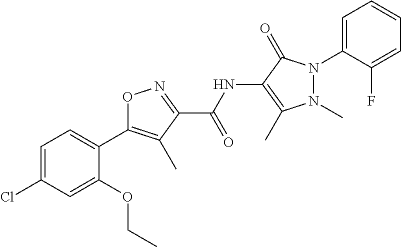 [SMILES: CCOc1cc(Cl)ccc1-c1onc(C(=O)Nc2c(C)n(C)n(-c3ccccc3F)c2=O)c1C] | 5-(4-Chloro-2- ethoxyphenyl)-N-(2-(2- fluorophenyl)-1,5- dimethyl-3-oxo-2,3- dihydro-1H-pyrazol-4- yl)-4-methylisoxazole- 3-carboxamide | LC-MS: Rt 1.24 min; m/z 485.2 [MH]⁺; Method 2minlowpHv02. |
| 062 | 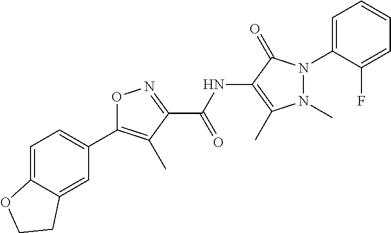 [SMILES: Cc1c(C(=O)Nc2c(C)n(C)n(-c3ccccc3F)c2=O)noc1-c1ccc2c(c1)CCO2] | 5-(2,3-Dihydrobenzo furan-5-yl)-N-(2-(2- fluorophenyl)-1,5- dimethyl-3-oxo-2,3- dihydro-1H-pyrazol-4- yl)-4-methylisoxazole- 3-carboxamide | LC-MS: Rt 1.09 min; m/z 449.2 [MH]⁺; Method 2minlowpHv02. |
| 063 | 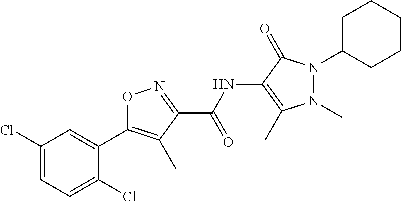 [SMILES: Cc1c(C(=O)Nc2c(C)n(C)n(C3CCCCC3)c2=O)noc1-c1cc(Cl)ccc1Cl] | N-(2-Cyclohexyl-1,5- dimethyl-3-oxo-2,3- dihydro-1H-pyrazol-4- yl)-5-(2,5- dichlorophenyl)-4- methylisoxazole-3- carboxamide | LC-MS: Rt 1.19 min; m/z 463.4 [MH]⁺; Method 2minHighpH. |
| 064 | 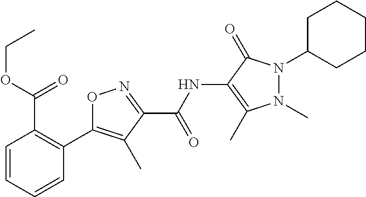 [SMILES: CCOC(=O)c1ccccc1-c1onc(C(=O)Nc2c(C)n(C)n(C3CCCCC3)c2=O)c1C] | Ethyl 2-(3-((2- cyclohexyl-1,5- dimethyl-3-oxo-2,3- dihydro-1H-pyrazol-4- yl)carbamoyl)-4- methylisoxazol-5- yl)benzoate | LC-MS: Rt 1.10 min; m/z 467.5 [MH]⁺; Method 2minHighpH. |
| 065 | 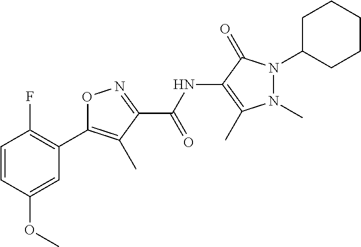 [SMILES: COc1ccc(F)c(-c2onc(C(=O)Nc3c(C)n(C)n(C4CCCCC4)c3=O)c2C)c1] | N-(2-Cyclohexyl-1,5- dimethyl-3-oxo-2,3- dihydro-1H-pyrazol-4- yl)-5-(2-fluoro-5- methoxyphenyl)-4- methylisoxazole-3- carboxamide | LC-MS: Rt 1.11 min; m/z 443.5 [MH]⁺; Method 2minHighpH. |
| 066 | 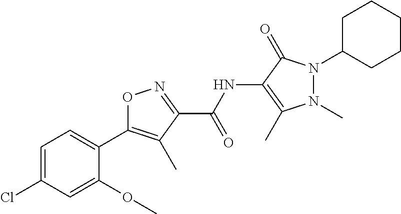 [SMILES: COc1cc(Cl)ccc1-c1onc(C(=O)Nc2c(C)n(C)n(C3CCCCC3)c2=O)c1C] | 5-(4-Chloro-2- methoxyphenyl)-N-(2- cyclohexyl-1,5- dimethyl-3-oxo-2,3- dihydro-1H-pyrazol-4- yl)-4-methylisoxazole- 3-carboxamide | LC-MS: Rt 1.10 mins; MS m/z 459.3 [M + H]+; Method 2minLowpH av67710: 1H NMR (400 MHz, DMSO-d6) d 9.60 (1H, s), 7.50 (1H, d), 7.33 (1H, d), 7.20 (1H, dd), 3.90 (1H, m), 3.88 (3H, s), 3.22 (3H, s), 2.05 (6H, s), 2.00 (2H, m), 1.79 (2H, m), 1.68 (3H, m), |
| | | | 1.32 (2H, m), 1.18 |
| | | | (1H, m). |
| 067 | 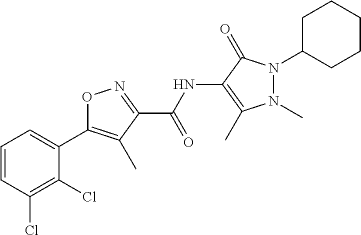 [SMILES: Cc1c(C(=O)Nc2c(C)n(C)n(C3CCCCC3)c2=O)noc1-c1cccc(Cl)c1Cl] | N-(2-Cyclohexyl-1,5- dimethyl-3-oxo-2,3- dihydro-1H-pyrazol-4- yl)-5-(2,3- dichlorophenyl)-4- methylisoxazole-3- carboxamide | LC-MS: Rt 1.18 min; m/z 465.4 [MH]⁺; Method 2minHighpH |
| 068 | 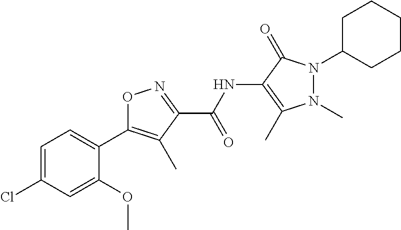 | 5-(4-Chloro-2- ethoxyphenyl)-N-(2- cyclohexyl-1,5- dimethyl-3-oxo-2,3- dihydro-1H-pyrazol-4- yl)-4-methylisoxazole- 3-carboxamide | LC-MS: Rt 1.16 mins; MS m/z 473.3 [M + H]+; Method 2minLowpH 1H NMR (400 MHz, DMSO-d6) d 9.60 (1H, s), 7.50 (1H, d), 7.32 (1H, s), 7.19 (1H, d), 4.18 (2H, q), 3.91 (1H, m), 3.22 (3H, s), 2.09 (3H, s), 2.05 (3H, s), 2.00 (2H, m), 1.80 (2H, m), 1.65 (3H, m), 1.32 (2H, m), 1.30 |
| | | | (3H, t), 1.18 (1H, m). |
| 069 | 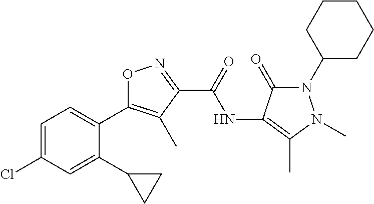 [SMILES: Cc1c(C(=O)Nc2c(C)n(C)n(C3CCCCC3)c2=O)noc1-c1ccc(Cl)cc1C1CC1] | 5-(4-Chloro-2- cyclopropylphenyl)-N- (2-cyclohexyl-1,5- dimethyl-3-oxo-2,3- dihydro-1H-pyrazol-4- yl)-4-methylisoxazole- 3-carboxamide | LC-MS: Rt 1.18 mins; MS m/z 469.3 [M + H]+; Method 2minLowpH 1H NMR (400 MHz, CDCl3) d 8.09 (1H, s), 7.26-7.25 (2H, m), 6.99 (1H, d), 4.13- 4.03 (1H, m), 3.28 (3H, s), 2.25 (3H, s), 2.21 (3H, s), 2.06- 1.97 (2H, m), 1.93- 1.82 (5H, m), 1.75- |
| | | | 1.69 (1H, m), 1.44- |
| | | | 1.34 (2H, m), 1.0-1.20 |
| | | | (1H, m), 0.98 (2H, q), |
| | | | 0.71 (2H, q). |
| 070 | 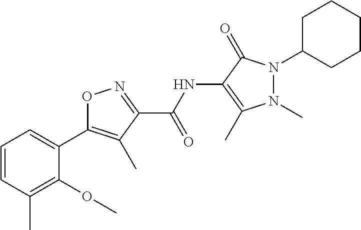 [SMILES: COc1c(C)cccc1-c1onc(C(=O)Nc2c(C)n(C)n(C3CCCCC3)c2=O)c1C] | N-(2-Cyclohexyl-1 ,5- dimethyl-3-oxo-2,3- dihydro-1H-pyrazol-4- yl)-5-(2-methoxy-3- methylphenyl)-4- methylisoxazole-3- carboxamide | LC-MS: Rt 1.05 mins; MS m/z 439.6 [M + H]+; Method 2minLowpH_TFA 1H NMR (400 MHz, DMSO-d6) δ 9.60 (1H, s), 7.45 (1H, d), 7.35 (1H, d), 7.22 (1H, dd), 3.94 (1H, m), 3.52 (3H, s), 3.24 (3H, s), 2.33 (3H, s), 2.11 (3H, s), 2.08 (3H, s), 2.02 (2H, m), 1.80 (2H, m), 1.65 (3H, m), |
| | | | 1.32 (2H, m), 1.18 |
| | | | (1H, m). |
| 071 | 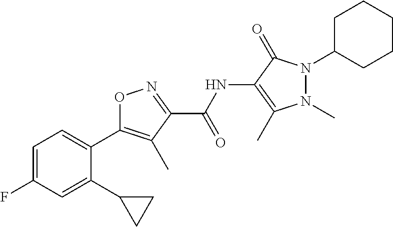 [SMILES: Cc1c(C(=O)Nc2c(C)n(C)n(C3CCCCC3)c2=O)noc1-c1ccc(F)cc1C1CC1] | N-(2-Cyclohexyl-1,5- dimethyl-3-oxo-2,3- dihydro-1H-pyrazol-4- yl)-5-(2-cyclopropyl-4- fluorophenyl)-4- methylisoxazole-3- carboxamide | LC-MSRt 1.14 mins; MS m/z 453.3 [M + H]+; Method 2minLowpH 1H NMR (400 MHz, DMSO-d6) δ 9.60 (1H, s), 7.45 (1H, dd), 7.18 (1H, dt), 6.91 (1H, dd), 3.91 (1H, m), 3.22 (3H, s), 2.09 (3H, s), 2.06 (3H, s), 2.00 (2H, m), 1.79 (3H, m), 1.68 (3H, m), 1.32 (2H, m), 1.18 |
| | | | (1H, m), 0.97 (2H, m), |
| | | | 0.79 (2H, m). |
| 072 | 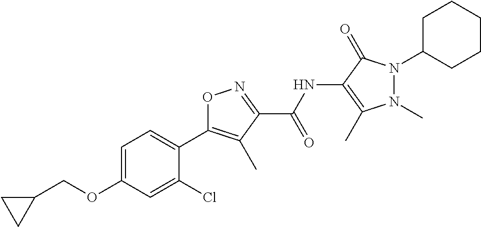 [SMILES: Cc1c(C(=O)Nc2c(C)n(C)n(C3CCCCC3)c2=O)noc1-c1ccc(OCC2CC2)cc1Cl] | 5-(2-Chloro-4-(cyclo propylmethoxy) phenyl)-N-(2- cyclohexyl-1,5- dimethyl-3-oxo-2,3- dihydro-1H-pyrazol-4- yl)-4-methylisoxazole- 3-carboxamide | LC-MS: Rt 1.20 mins; MS m/z 499.4 [M + H]+; Method 2minLowpH 1H NMR (400 MHz, DMSO-d6) d 9.60 (1H, s), 7.51 (1H, d), 7.27 (1H, d), 7.10 (1H, dd), 3.95 (2H, d), 3.92 (1H, m), 3.22 (3H, s), 2.08 (6H, s), 2.02 (2H, m), 1.80 (2H, m), 1.65 (3H, m), 1.40-1.15 (4H, m), 0.60 (2H , |
| | | | m), 0.37 (2H, m). |
| 073 | 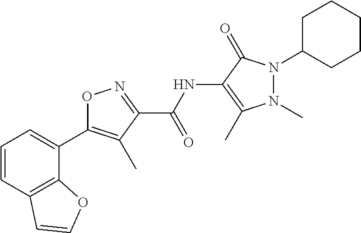 [SMILES: Cc1c(C(=O)Nc2c(C)n(C)n(C3CCCCC3)c2=O)noc1-c1cccc2ccoc12] | 5-(Benzofuran-7-yl)-N- (2-cyclohexyl-1,5- dimethyl-3-oxo-2,3- dihydro-1H-pyrazol-4- yl)-4-methylisoxazole- 3-carboxamide | LC-MS: Rt 1.05 mins; MS m/z 435.3 [M + H]+; Method 2minLowpH 1H NMR (400 MHz, DMSO-d6) d 9.65 (1H, s), 8.18 (1H, d), 7.91 (1H, d), 7.64 (1H, d), 7.48 (1H, dd), 7.14 (1H, d), 3.92 (1H, m), 3.24 (3H, s), 2.29 (3H, s), 2.08 (3H, s), 2.00 (2H, m), 1.80 (2H, m), 1.65 (3H, m), 1.32 |
| | | | (2H, m), 1.18 (1H, m). |
| 074 | 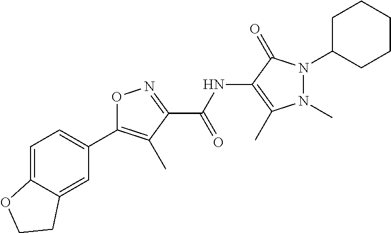 [SMILES: Cc1c(C(=O)Nc2c(C)n(C)n(C3CCCCC3)c2=O)noc1-c1ccc2c(c1)CCO2] | N-(2-Cyclohexyl-1,5- dimethyl-3-oxo-2,3- dihydro-1H-pyrazol-4- yl)-5-(2,3-dihydrobenzo furan-5-yl)-4-methyl isoxazole-3- carboxamide | LC-MS: Rt 1.04 mins; MS m/z 438.3 [M + H]+; Method 2minLowpH 1H NMR (400 MHz, DMSO-d6) d 9.55 (1H, s), 7.65 (1H, s), 7.52 (1H, d), 6.97 (1H, d), 4.63 (2H, t), 4.02 (1H, m), 3.30 (2H, t), 3.21 (3H, s), 2.30 (3H, s), 2.08 (3H, s), 2.00 (2H, m), 1.80 (2H, m), 1.65 (3H, m), 1.30 |
| | | | (2H, m), 1.16 (1H, m). |
| 075 | 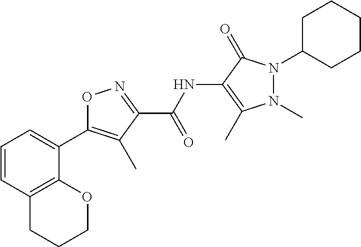 [SMILES: Cc1c(C(=O)Nc2c(C)n(C)n(C3CCCCC3)c2=O)noc1-c1cccc2c1OCCC2] | 5-(Chroman-8-yl)-N-(2- cyclohexyl-1,5- dimethyl-3-oxo-2,3- dihydro-1H-pyrazol-4- yl)-4-methyl isoxazole- 3-carboxamide | LC-MS: Rt 1.06 mins; MS m/z 451.6 [M + H]+; Method 2minLowpH_TFA. 1H NMR (400 MHz, DMSO-d6) d 9.60 (1H, s), 7.28 (2H, m), 6.98 (1H, t), 4.21 (2H, m), 3.91 (1H, m), 3.22 (3H, s), 2.82 (2H, m), 2.07 (6H, s), 2.00 (4H, m), 1.80 (2H, m), 1.65 (3H, m), 1.32 (2H, m), 1.18 (1H, m). |
| 076 | 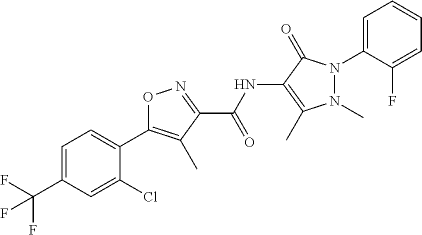 [SMILES: Cc1c(C(=O)Nc2c(C)n(C)n(-c3ccccc3F)c2=O)noc1-c1ccc(C(F)(F)F)cc1Cl] | 5-(2-chloro-4- (trifluoromethyl)phenyl)- N-(2-(2-fluorophenyl)- 1,5-dimethyl-3-oxo-2,3- dihydro-1H-pyrazol-4- yl)-4-methylisoxazole- 3-carboxamide | Rt 1.24 mins; MS m/z 509.1 [M + H]+; Method 2minLowpH |
| 077 | 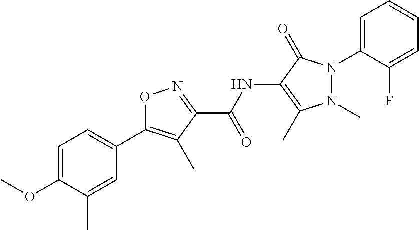 [SMILES: COc1ccc(-c2onc(C(=O)Nc3c(C)n(C)n(-c4ccccc4F)c3=O)c2C)cc1C] | N-(2-(2-fluorophenyl)- 1,5-dimethyl-3-oxo-2,3- dihydro-1H-pyrazol-4- yl)-5-(4-methoxy-3- methylphenyl)-4- methylisoxazole-3- carboxamide | Rt 1.17 mins; MS m/z 451.2 [M + H]+; Method 2minLowpH |
| 078 | 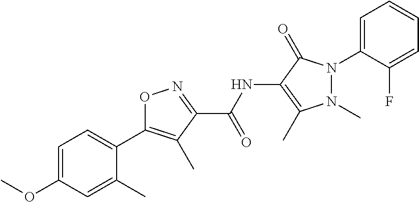 [SMILES: COc1ccc(-c2onc(C(=O)Nc3c(C)n(C)n(-c4ccccc4F)c3=O)c2C)c(C)c1] | N-(2-(2-fluorophenyl)- 1,5-dimethyl-3-oxo-2,3- dihydro-1H-pyrazol-4- yl)-5-(4-methoxy-2- methylphenyl)-4- methylisoxazole-3- carboxamide | Rt 1.13 mins; MS m/z 451.2 [M + H]+; Method 2minLowpH |
| 079 | 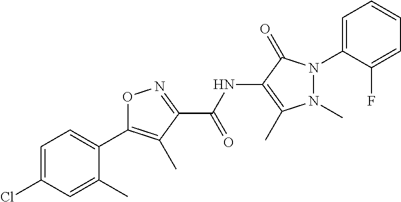 [SMILES: Cc1cc(Cl)ccc1-c1onc(C(=O)Nc2c(C)n(C)n(-c3ccccc3F)c2=O)c1C] | 5-(4-chloro-2- methylphenyl)-N-(2-(2- fluorophenyl)-1,5- dimethyl-3-oxo-2,3- dihydro-1H-pyrazol-4- yl)-4-methylisoxazole- 3-carboxamide | Rt 1.22 mins; MS m/z 455.2 [M + H]+; Method 2minLowpH |
| 080 | 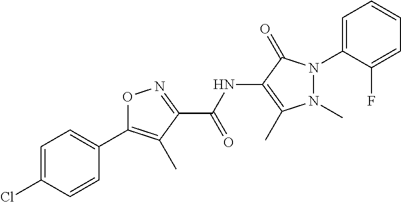 [SMILES: Cc1c(C(=O)Nc2c(C)n(C)n(-c3ccccc3F)c2=O)noc1-c1ccc(Cl)cc1] | 5-(4-chlorophenyl)-N- (2-(2-fluorophenyl)-1,5- dimethyl-3-oxo-2,3- dihydro-1H-pyrazol-4- yl)-4-methylisoxazole- 3-carboxamide | Rt 1.17 mins; MS m/z 441.1 [M + H]+; Method 2minLowpH |
| 081 | 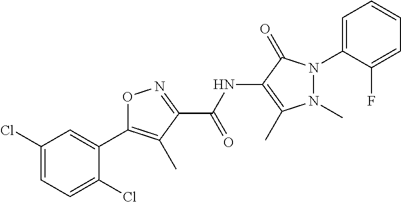 [SMILES: Cc1c(C(=O)Nc2c(C)n(C)n(-c3ccccc3F)c2=O)noc1-c1cc(Cl)ccc1Cl] | 5-(2,5-dichlorophenyl)- N-(2-(2-fluorophenyl)- 1,5-dimethyl-3-oxo-2,3- dihydro-1H-pyrazol-4- yl)-4-methylisoxazole- 3-carboxamide | Rt 1.2 mins; MS m/z 475.1 [M + H]+; Method 2minLowpH |
| 082 | 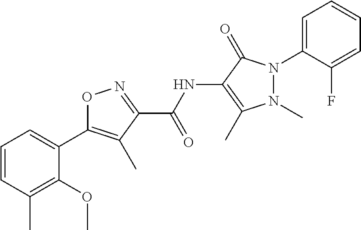 [SMILES: COc1c(C)cccc1-c1onc(C(=O)Nc2c(C)n(C)n(-c3ccccc3F)c2=O)c1C] | N-(2-(2-fluorophenyl)- 1,5-dimethyl-3-oxo-2,3- dihydro-1H-pyrazol-4- yl)-5-(2-methoxy-3- methylphenyl)-4- methylisoxazole-3- carboxamide | Rt 1.14 mins; MS m/z 451.2 [M + H]+; Method 2minLowpH |
| 083 | 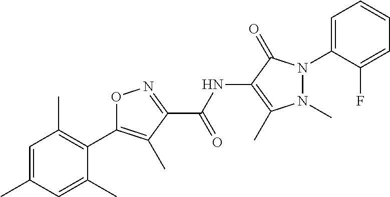 [SMILES: Cc1cc(C)c(-c2onc(C(=O)Nc3c(C)n(C)n(-c4ccccc4F)c3=O)c2C)c(C)c1] | N-(2-(2-fluorophenyl)- 1,5-dimethyl-3-oxo-2,3- dihydro-1H-pyrazol-4- yl)-5-mesityl-4- methylisoxazole-3- carboxamide | 449.2 [M + H]+; Method Rt 1.24 mins; MS m/z 2minLowpH |
| 084 | 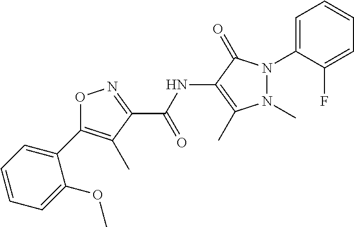 [SMILES: COc1ccccc1-c1onc(C(=O)Nc2c(C)n(C)n(-c3ccccc3F)c2=O)c1C] | N-(2-(2-fluorophenyl)- 1,5-dimethyl-3-oxo-2,3- dihydro-1H-pyrazol-4- yl)-5-(2- methoxyphenyl)-4- methylisoxazole-3- carboxamide | Rt 1.08 mins; MS m/z 437.2[M + H]+; Method 2minLowpH |

### Example 085

### N-(2-Cyclohexyl-1,5-dimethyl-3-oxo-2,3-dihydro-1H-pyrazol-4-yl)-4-cyclopropyl-5-(4-methoxy-2-(trifluoro methyl)phenyl)isoxazole-3-carboxamide

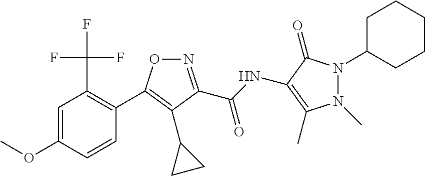 [SMILES: COc1ccc(-c2onc(C(=O)Nc3c(C)n(C)n(C4CCCCC4)c3=O)c2C2CC2)c(C(F)(F)F)c1]

To phosgene (0.033 g, 0.26 mmol) in DMF (0.035 g) and DCM (5 ml) was added 4-cyclopropyl-5-(4-methoxy-2-(trifluoromethyl)phenyl)isoxazole-3-carboxylic acid (Intermediate L75b) (0.08 g, 0.24 mmol) and the mixture was stirred for 5 mins. 4-Amino-2-cyclohexyl-1,5-dimethyl-1H-pyrazol-3(2H)-one (commercially available) (0.05 g, 0.24 mmol) and triethylamine (0.072 g, 0.71 mmol) were added and the resulting mixture was stirred for 30 mins. The resulting mixture was partitioned between NaHCO₃ and DCM and extracted with DCM. The combined organic extracts were dried and concentrated in vacuo. Purification by chromatography on silica eluting with 4:6 EtOAc/hexane afforded the title compound;

LC-MS: m/z 519.2 [M+H]⁺.

### Example 086

### N-(2-Cyclohexyl-1,5-dimethyl-3-oxo-2,3-dihydro-1H-pyrazol-4-yl)-4-cyclopropyl-5-(2-fluoro-4-methoxyphenyl) isoxazole-3-carboxamide

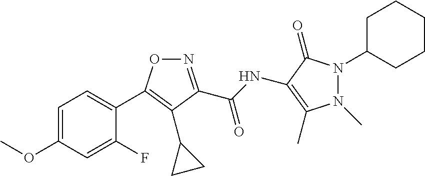 [SMILES: COc1ccc(-c2onc(C(=O)Nc3c(C)n(C)n(C4CCCCC4)c3=O)c2C2CC2)c(F)c1]

The title compound was prepared from 4-cyclopropyl-5-(2-fluoro-4-methoxyphenyl)isoxazole-3-carboxylic acid (Intermediate L76b) and 4-amino-2-cyclohexyl-1,5-dimethyl-1H-pyrazol-3(2H)-one analogously (Commercial) to Example 85; LC-MS: m/z 469.2 [M+H]⁺.

### Example 087

### 4-Cyclopropyl-N-(2-(2-fluorophenyl)-1,5-dimethyl-3-oxo-2,3-dihydro-1H-pyrazol-4-yl)-5-(4-methoxy-2-(trifluoromethyl)phenyl)isoxazole-3-carboxamide

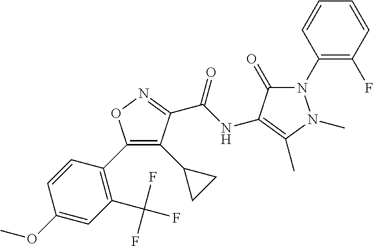 [SMILES: COc1ccc(-c2onc(C(=O)Nc3c(C)n(C)n(-c4ccccc4F)c3=O)c2C2CC2)c(C(F)(F)F)c1]

The title compound was prepared from 4-cyclopropyl-5-(4-methoxy-2-(trifluoromethyl)phenyl)isoxazole-3-carboxylic acid and 4-amino-2-(2-fluorophenyl)-1,5-dimethyl-1H-pyrazol-3(2H)-one (commercial) analogously to Example 003

LC-MS: m/z 531.2 [M+H]⁺.

Method 10minLowpHv01

### Preparation of Intermediates

### Intermediate L01

### 5-(2,4-Dichloro-phenyl)-4-methyl-isoxazole-3-carboxylic acid

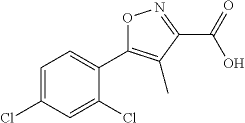 [SMILES: Cc1c(C(=O)O)noc1-c1ccc(Cl)cc1Cl]

### Intermediate L01 Step 1

(Z)-4-(2,4-Dichloro-phenyl)-4-hydroxy-3-methyl-2-oxo-but-3-enoic acid ethyl ester LiHMDS (27.1 ml, 27.1 mmol) and 120 ml diethyl ether wee stirred at −78° C. Then 2, 4-dichloropropiophenone (5 g, 24.62 mmol) dissolved in 24 ml diethyl ether was added dropwise. After 45 min, diethyl oxalate (4.37 ml, 32.0 mmol) was added. The mixture was stirred for 1 h at RT, then diluted with DCM and washed with water. The combined organic layers were dried over MgSO₄ and evaporated to dryness. LC-MS: Rt 1.16 min; m/z 302.9[MH]; Method B.

### Intermediate L01 Step 2

### 5-(2,4-Dichloro-phenyl)-4-methyl-isoxazole-3-carboxylic acid ethyl ester

(Z)-4-(2, 4-Dichloro-phenyl)-4-hydroxy-3-methyl-2-oxo-but-3-enoic acid ethyl ester (5.6 g, 18.47 mmol) and hydroxylamine hydrochloride (1.54 g, 22.17 mmol) were stirred with 100 ml EtOH at 80° C. After 16 h, the mixture was allowed to reach RT. Purification by chromatography on silica gel (hexane/ethyl acetate) afforded 5-(2,4-dichloro-phenyl)-4-methyl-isoxazole-3-carboxylic acid ethyl ester with 20% purity.

### Intermediate L01 Step 3

### 5-(2,4-Dichloro-phenyl)-4-methyl-isoxazole-3-carboxylic acid

5-(2,4-Dichloro-phenyl)-4-methyl-isoxazole-3-carboxylic acid ethyl ester (1.12 g, 0.746 mmol, 20%) and NaOH 2 mol/l (0.373 ml, 0.746 mmol) was stirred in 20 ml MeOH over night at RT. The reaction mixture was extracted with DCM and NaOH. The inorganic phase was acidified with HCl (37%) and extracted two times with dichloromethane, dried over MgSO₄ and evaporated to dryness. LC-MS: Rt 0.97 min; no mass detected; Method B.

### Intermediate L02

### 5-(2-Chloro-4-cyclopropyl-phenyl)-4-methyl-isoxazole-3-carboxylic acid

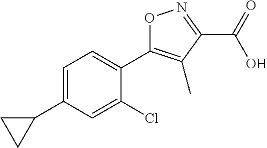 [SMILES: Cc1c(C(=O)O)noc1-c1ccc(C2CC2)cc1Cl]

LC-MS: Rt 2.16 min; no mass detected: Method A.

### Intermediate L02 Step 1

### 2-Chloro-4-cyclopropyl-benzoic acid ethyl ester

4-Bromo-2-chloro-benzoic acid ethyl ester (17.7 g, 67.2 mmol), cyclopropyl boronic acid monohydrate (8.65 g, 101 mmol), Pd(OAC)₂ (0.754 g, 3.36 mmol), potassium phosphate (49.9 g, 235 mmol) and triphenylphosphine (1.762 g, 6.72 mmol) were stirred in 100 ml toluene, then 15 ml H₂O was added. The mixture was stirred at 100° C. for 16 h, then cooled to RT, diluted with ethyl acetate and washed with water (3 times). The organic layer was dried over MgSO₄ and evaporated to dryness. Purification by chromatography on silica gel (hexane/ethyl acetate) afforded 15.17 g (54 mmol, 80%) of 2-chloro-4-cyclopropyl-benzoic acid ethyl ester. LC-MS: Rt 2.35 min; no mass detected; Method A.

### Intermediate L02 Step 2

### 2-Chloro-4-cyclopropyl-benzoic acid

2-Chloro-4-cyclopropyl-benzoic acid ethyl ester (15.17 g, 54 mmol) and NaOH 32% (12.51 ml 135 mmol) were stirred in MeOH (150 ml) at RT for 1.5 h. The mixture was distributed between 1 M NaOH and DCM, the phases separated and the inorganic layer acidified with HCl (37%). Extraction with DCM (3×), drying with MgSO₄ and evaporation afforded the crude product that was used without further purification.

Rt 1.94 min; no mass detected; Method A.

### Intermediate L02 Step 3

### 2-Chloro-4-cyclopropyl-N-methoxy-N-methyl-benzamide

DMF (13.07 ml, 169 mmol) was added to DCM (300 ml) and cooled to 0° C. Oxalyl chloride (8.13 ml, 93 mmol) and 2-chloro-4-cyclopropyl-benzoic acid (8.3 g, 42.2 mmol) dissolved in DCM (100 ml) were added. After stirring for 15 min, N, O-dimethyl hydroxylamine hydrochloride (8.14 g, 84 mmol) and triethylamine (35.3 ml, 253 mmol) were added. The mixture was stirred at 0° and then allowed to reach RT within 45 min. Evaporation and purification by chromatography on silica gel (hexane/ethyl acetate) afforded 2-chloro-4-cyclopropyl-N-methoxy-N-methyl-benzamide as a light yellow oil.

LC-MS: Rt 1.98 min; m/z 240[MH]⁺; Method A.

### Intermediate L02 Step 4

### 1-(2-Chloro-4-cyclopropyl-phenyl)-propan-1-one

2-Chloro-4-cyclopropyl-N-methoxy-N-methyl-benzamide (6.5 g, 27.1 mmol) was dissolved in 200 ml THF. EtMgBr (1 M, 59.7 ml, 59.7 mmol) was added slowly at RT. After 7 h the mixture was diluted with DCM and washed with water. The organic layer was dried over MgSO₄ and evaporated to dryness. Purification by chromatography on silica gel (hexane/ethyl acetate) afforded 1-(2-chloro-4-cyclopropyl-phenyl)-propan-1-one.

Rt 2.32 min; no mass detected; Method A.

The remaining steps towards intermediate L02 were performed by similar methods to intermediate L01 steps 1 to 3.

The following intermediates (Table 2) were prepared in a similar fashion to intermediate L02:

TABLE 2

| Int | Structure | Name | Analytical data |
|---|---|---|---|
| L03 | 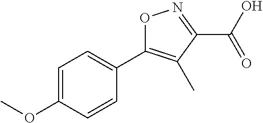 [SMILES: COc1ccc(-c2onc(C(=O)O)c2C)cc1] | 5-(4-Methoxy-phenyl)-4- methyl-isoxazole-3-carboxylic acid | LC-MS: Rt 1.87 min; m/z 234[M + H]⁺; Method A |
| L04 | 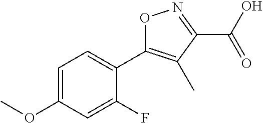 [SMILES: COc1ccc(-c2onc(C(=O)O)c2C)c(F)c1] | 5-(2-Fluoro-4-methoxy- phenyl)-4-methyl-isoxazole-3- carboxylic acid | LC-MS: Rt 1.91 min; no mass detected; Method A |
| L05 | 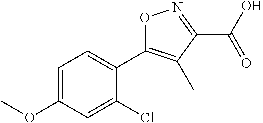 [SMILES: COc1ccc(-c2onc(C(=O)O)c2C)c(Cl)c1] | 5-(2-Chloro-4-methoxy- phenyl)-4-methyl-isoxazole-3- carboxylic acid | LC-MS: Rt 1.97 min; no mass detected; Method A |
| L06 | 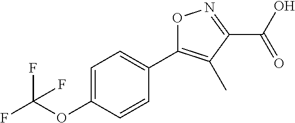 [SMILES: Cc1c(C(=O)O)noc1-c1ccc(OC(F)(F)F)cc1] | 4-Methyl-5-(4- trifluoromethoxy-phenyl)- isoxazole-3-carboxylic acid | LC-MS: Rt 2.13 min; no mass detected; Method A |
| L07 | 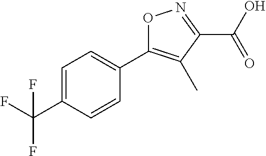 [SMILES: Cc1c(C(=O)O)noc1-c1ccc(C(F)(F)F)cc1] | 4-Methyl-5-(4-trifluoromethyl- phenyl)-isoxazole-3- carboxylic acid | LC-MS: Rt 1.95 min; no mass detected; Method A |
| L08 | 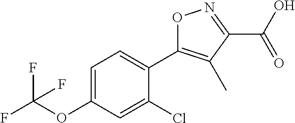 [SMILES: Cc1c(C(=O)O)noc1-c1ccc(OC(F)(F)F)cc1Cl] | 5-(2-Chloro-4- trifluoromethoxy-phenyl)-4- methyl-isoxazole-3-carboxylic acid | LC-MS: Rt 1.04 min; no mass detected; Method B |
| L09 | 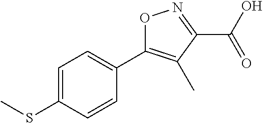 [SMILES: CSc1ccc(-c2onc(C(=O)O)c2C)cc1] | 4-Methyl-5-(4-methylsulfanyl- phenyl)-isoxazole-3- carboxylic acid | LC-MS: Rt 2.02 min; no mass detected; Method A |
| L10 | 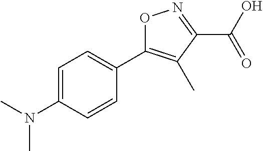 [SMILES: Cc1c(C(=O)O)noc1-c1ccc(N(C)C)cc1] | 5-(4-Dimethyl amino-phenyl)- 4-methyl-isoxazole-3- carboxylic acid | LC-MS: Rt 1.61 min; no mass detected; Method A |
| L11 | 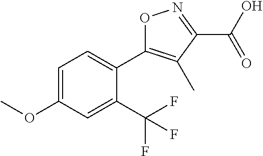 [SMILES: COc1ccc(-c2onc(C(=O)O)c2C)c(C(F)(F)F)c1] | 5-(4-Methoxy-2- trifluoromethyl-phenyl)-4- methyl-isoxazole-3-carboxylic acid | LC-MS: Rt 2.06 min; no mass detected; Method A |
| L12 | 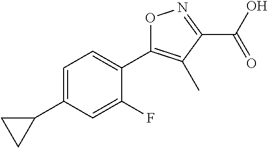 [SMILES: Cc1c(C(=O)O)noc1-c1ccc(C2CC2)cc1F] | 5-(4-Cyclopropyl-2-fluoro- phenyl)-4-methyl-isoxazole-3- carboxylic acid | LC-MS: Rt 2.11 min; no mass detected; Method A |

### Intermediate L13

### 4-Chloro-5-(2-chloro-4-difluoromethoxy-phenyl)-isoxazole-3-carboxylic acid

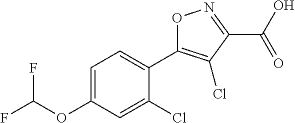 [SMILES: O=C(O)c1noc(-c2ccc(OC(F)F)cc2Cl)c1Cl]

### Intermediate L13 Step 1

### 1-Bromo-2-chloro-4-difluoromethoxy-benzene

4-Bromo-3-chlorophenole (5 g, 24.10 mmol), sodium chlorodifluoroacetate (7.35 g, 48.2 mmol) and K₂CO₃ were dissolved in DMF (55 ml) and stirred at 80° C. After 2 h, the mixture was diluted with water and extracted with DCM, dried with MgSO₄ and evaporated to dryness. Purification by chromatography on silica gel (hexane/ethyl acetate) afforded the product as a colorless oil. NMR (400 MHz CDCl3) δ 7.6 (d, 1H) 7.27 (d, 1H) 6.93 (dd, 1H) 6.49 (t, 1H, 72.63 Hz).

### Intermediate L13 Step 2

### 1-(2-Chloro-4-difluoromethoxy-phenyl)-ethanone

1-Bromo-2-chloro-4-difluoromethoxy-benzene (2 g, 7.77 mmol) was dissolved in dioxane. Tributyl(1-ethoxyvinyl) tin (2.88 ml, 8.55 mmol) and Pd(Ph₃P)₄ (0.748 g, 0.647 mmol) were added. The mixture was stirred at 110° C. for 2 h. The reaction was cooled to room temperature over night. HCl 2 M (12.82 ml, 25.6 mmol) was added. After 1 h, the mixture was added to an aqueous solution of KF. Insoluble solids were filtered off, the filtrate was extracted with DCM, the DCM extracts dried over MgSO₄ and evaporated to dryness. Purification by chromatography on silica gel (hexane/ethyl acetate) afforded 1-(2-chloro-4-difluoromethoxy-phenyl)-ethanone.

NMR (400 MHz CDCl3) δ 7.65 (d, 1H) 7.20 (d, 1H) 7.08 (dd, 1H) 6.56 (t, 1H, 72.63 Hz) 2.63 (s, 3H).

### Intermediate L13 Step 3

### 4-(2-Chloro-4-difluoromethoxy-phenyl)-2,4-dioxo-butyric acid ethyl ester

Sodium ethanolate (2.57 ml, 6.98 mmol) was dissolved in EtOH (10 ml) and 1-(2-chloro-4-difluoromethoxy-phenyl)-ethanone (0.77 g, 3.49 mmol) was added. The mixture stirred at RT for 10 min. Diethyl oxalate (0.763 ml, 5.58 mmol) was added and the mixture stirred at 80° C. for 30 min. The mixture was diluted with water and 1M HCl, then extracted with DCM, dried with MgSO₄ and evaporated to dryness to give 4-(2-chloro-4-difluoromethoxy-phenyl)-2,4-dioxo-butyric acid ethyl ester.

LC-MS: Rt 2.31 min; no mass detected; Method A.

### Intermediate L13 Step 4

### 5-(2-Chloro-4-difluoromethoxy-phenyl)-isoxazole-3-carboxylic acid ethyl ester

4-(2-Chloro-4-difluoromethoxy-phenyl)-2,4-dioxo-butyric acid ethyl ester (1.0 g, 3.12 mmol) was dissolved in EtOH (15 ml), then hydroxylamine hydrochloride (0.26 g, 3.74 mmol) was added. The mixture was stirred at 80° C. for 1 h. The resulting white suspension was filtered off and washed with water to give 5-(2-chloro-4-difluoromethoxy-phenyl)-isoxazole-3-carboxylic acid ethyl ester as a white solid.

LC-MS: Rt 2.34 min; no mass detected; Method A.

### Intermediate L13 Step 5

### 4-Chloro-5-(2-chloro-4-difluoromethoxy-phenyl)-isoxazole-3-carboxylic acid ethyl ester

5-(2-Chloro-4-difluoromethoxy-phenyl)-isoxazole-3-carboxylic acid ethyl ester (634 mg, 1.996 mmol) and NCS (533 mg, 3.99 mmol) were dissolved in glacial acetic acid and stirred at 100° C. for 72 h. The reaction mixture was diluted with DCM and washed with water. The organic layer was dried over MgSO₄ and evaporated to dryness. Purification by reversed phase chromatography on a sunfire column afforded 4-chloro-5-(2-chloro-4-difluoromethoxy-phenyl)-isoxazole-3-carboxylic acid ethyl ester.

Rt 2.41 min; no mass detected; Method A.

### Intermediate L13 Step 6

### 4-Chloro-5-(2-chloro-4-difluoromethoxy-phenyl)-isoxazole-3-carboxylic acid

4-Chloro-5-(2-chloro-4-difluoromethoxy-phenyl)-isoxazole-3-carboxylic acid ethyl ester (130 mg, 0.332 mmol) was dissolved in MeOH and 2 M NaOH (0.166 ml, 0.332 mmol) was added. The mixture was stirred for 1 h at RT. The solution was acidified with 2 M HCl and extracted with DCM. The organic layer was dried over MgSO₄ and evaporated to dryness.

Rt 2.07 min; no mass detected; Method A.

The following intermediates (Table 3) were prepared analogously to intermediate L13 utilising the appropriate halogenating agent

TABLE 3

| Int | Structure | Name | Analytical data |
|---|---|---|---|
| L14 | 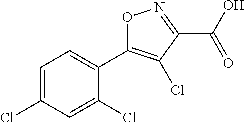 [SMILES: O=C(O)c1noc(-c2ccc(Cl)cc2Cl)c1Cl] | 4-Chloro-5-(2,4-dichloro- phenyl)-isoxazole-3- carboxylic acid | LC-MS: Rt 2.16 min; no mass detected; Method A |
| L15 | 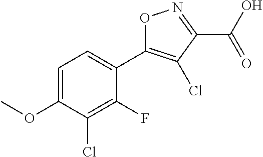 [SMILES: COc1ccc(-c2onc(C(=O)O)c2Cl)c(F)c1Cl] | 4-Chloro-5-(3-chloro-2- fluoro-4-methoxy-phenyl)- isoxazole-3-carboxylic acid | LC-MS: Rt 2.04 min; no mass detected; Method A |
| L16 | 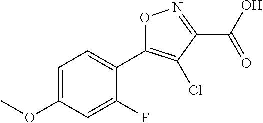 [SMILES: COc1ccc(-c2onc(C(=O)O)c2Cl)c(F)c1] | 4-Chloro-5-(2-fluoro-4- methoxy-phenyl)- isoxazole-3-carboxylic acid | LC-MS: Rt 2.31 min; no mass detected; Method A |
| L17 | 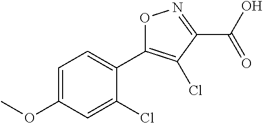 [SMILES: COc1ccc(-c2onc(C(=O)O)c2Cl)c(Cl)c1] | 4-Chloro-5-(2-chloro-4- methoxy-phenyl)- isoxazole-3-carboxylic acid | LC-MS: Rt 2.03 min; no mass detected [M + H]; Method A |
| L18 | 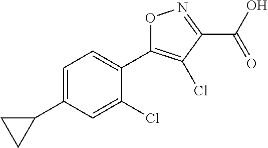 [SMILES: O=C(O)c1noc(-c2ccc(C3CC3)cc2Cl)c1Cl] | 4-Chloro-5-(2-chloro-4- cyclopropyl-phenyl)- isoxazole-3-carboxylic acid | LC-MS: Rt 2.19 min; no mass detected; Method A |
| L75a | 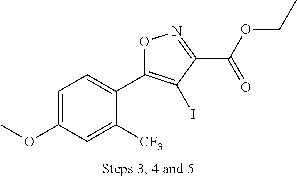 [SMILES: CCOC(=O)c1noc(-c2ccc(OC)cc2C(F)(F)F)c1I] | Ethyl 4-iodo-5-(4-methoxy- 2- (trifluoromethyl)phenyl) isoxazole-3-carboxylate | MS m/z 442 [M + H]+ |
| L76a | 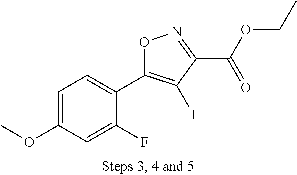 [SMILES: CCOC(=O)c1noc(-c2ccc(OC)cc2F)c1I] | Ethyl 5-(2-fluoro-4- methoxyphenyl)-4- iodoisoxazole-3-carboxylate | MS m/z 391.9 [M + H]+ |

### Intermediate L19

### 5-(2-Chloro-4-trifluoromethoxy-phenyl)-isoxazole-3-carboxylic acid

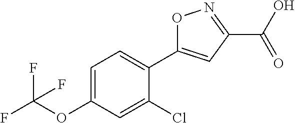 [SMILES: O=C(O)c1cc(-c2ccc(OC(F)(F)F)cc2Cl)on1]

Intermediate L19 was prepared from 1-(2 Chloro-4-trifluoromethoxy)phenyl)ethanone by a method similar to intermediate L13 steps 3,4 and 6 LC-MS: Rt 2.17 min, no mass detected; Method A.

### Intermediate L20

### 5-(2-Chloro-4-methoxy-phenyl)-4-fluoro-isoxazole-3-carboxylic acid

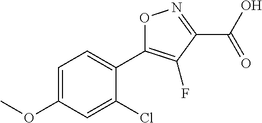 [SMILES: COc1ccc(-c2onc(C(=O)O)c2F)c(Cl)c1]

LC-MS: Rt 1.95 min, m/z no mass detected [M+H]⁺; Method A

### Intermediate L20 Step 2

### Ethyl 5-(2-chloro-4-methoxyphenyl)isoxazole-3-carboxylate

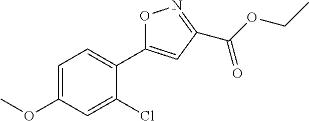 [SMILES: CCOC(=O)c1cc(-c2ccc(OC)cc2Cl)on1]

Intermediate L20 was prepared from 1-(2-chloro-4-methoxyphenyl) ethanone by a similar method to that of intermediate L13 step 3 and 4.

### Intermediate L20 Step 3

### 5-(2-Chloro-4-methoxy-phenyl)-4-fluoro-isoxazole-3-carboxylic acid ethyl ester

Ethyl-5-(2-chloro-4-methoxyphenyl)isoxazole-3-carboxylate (600 mg, 2.213 mmol) was dissolved in ACN (10 mL) and Selectfluor® (792 mg, 2.236 mmol) was added. The reaction was stirred at reflux for 7 days. The reaction was evaporated to dryness. The residue was purified by reversed phase chromatography on a sunfire column to give 5-(2-chloro-4-methoxy-phenyl)-4-fluoro-isoxazole-3-carboxylic acid ethyl ester as a pale off-white solid. LC-MS: Rt 2.29 min; m/z no mass detected [MH]⁺; Method A.

The remaining step towards intermediate L20 was performed by a similar Method to intermediate L13 step 6.

### Intermediate L21

### 4-Bromo-5-(2-chloro-4-methoxy-phenyl)-isoxazole-3-carboxylic acid

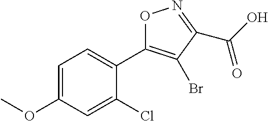 [SMILES: COc1ccc(-c2onc(C(=O)O)c2Br)c(Cl)c1]

LC-MS: Rt 2.02 min, m/z no mass detected [M+H]⁺; Method A.

### Intermediate L21 Step 1

### 4-Bromo-5-(2-chloro-4-methoxy-phenyl)-isoxazole-3-carboxylic acid ethyl ester

Ethyl-5-(2-chloro-4-methoxyphenyl)isoxazole-3-carboxylate (1.0 g, 3.12 mmol) (intermediate L20 step 1) was dissolved in acetic acid (10 ml), NBS (556 mg, 3.16 mmol) was added and the reaction stirred under microwave irradiation for 1 h. The resulting mixture was purified by chromatography on silica gel (hexane/ethyl acetat) followed by prep HPLC on a sunfire column to give 4-bromo-5-(2-chloro-4-methoxy-phenyl)-isoxazole-3-carboxylic acid ethyl ester as a white solid. LC-MS: Rt 2.36 min; m/z no mass detected [MH]⁺; Method A.

The remaining step towards intermediate L21 was performed by a similar Method to intermediate L13 step 6.

### Intermediate L22

### 4-Bromo-5-(2,4-dichloro-phenyl)-isoxazole-3-carboxylic acid

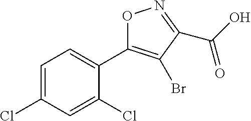 [SMILES: O=C(O)c1noc(-c2ccc(Cl)cc2Cl)c1Br]

Intermediate L22 was prepared by a similar Method to that of intermediate L21, starting from 1-(2,4-dichlorophenyl)ethanone LC-MS: Rt 2.17 min, m/z no mass detected [M+H]⁺; Method A.

### Intermediate L23

### 5-(2-Chloro-4-methoxy-phenyl)-4-cyclopropyl-isoxazole-3-carboxylic acid

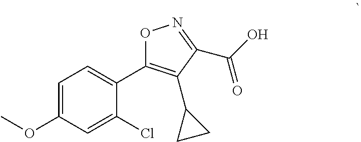 [SMILES: COc1ccc(-c2onc(C(=O)O)c2C2CC2)c(Cl)c1]

LC-MS: Rt 2.07 min, m/z no mass detected [M+H]⁺; Method A.

### Intermediate L23 Step 1

### 5-(2-Chloro-4-methoxy-phenyl)-4-cyclopropyl-isoxazole-3-carboxylic acid ethyl ester

4-Bromo-5-(2-chloro-4-methoxy-phenyl)-isoxazole-3-carboxylic acid ethyl ester (Intermediate L21 step 1, 200 mg, 0.555 mmol) was dissolved in toluene (1 ml), and cyclopropyl boronic acid (119 mg, 1.387 mmol) and triphenylphospine (29 mg, 0.111 mmol) were added. Potassium phosphate (2M, 1.6 ml, 3.33 mmol) was added followed by palladium (II) acetate (13 mg, 0.055 mmol). The reaction mixture was stirred at 100° C. for 1.5 h. The reaction was cooled to room temperature, diluted with water and extracted with dichloromethane. The combined organic layers were dried and evaporated to dryness. The crude material was purified by prep HPLC on a sunfire column to give 5-(2-chloro-4-methoxy-phenyl)-4-cyclopropyl-isoxazole-3-carboxylic acid ethyl ester. LC-MS: Rt 2.42 min; m/z 322.0 [MH]⁺; Method A.

The remaining step towards intermediate L23 was performed by a similar method to intermediate L13 step 6.

### Intermediate L24

### 4-Cyclopropyl-5-(2,4-dichloro-phenyl)-isoxazole-3-carboxylic acid

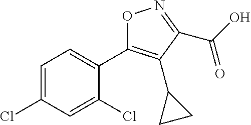 [SMILES: O=C(O)c1noc(-c2ccc(Cl)cc2Cl)c1C1CC1]

Intermediate L24 was prepared by a similar method to that of Intermediate L23, starting from 4-Bromo-5-(2,4-dichloro-phenyl)-isoxazole-3-carboxylic acid

LC-MS: Rt 2.54 min, m/z no mass detected [M+H]⁺; Method A.

### Intermediate L25

### 4-Cyano-5-(2,4-dichloro-phenyl)-isoxazole-3-carboxylic acid

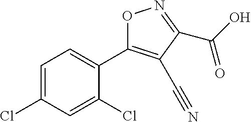 [SMILES: N#Cc1c(C(=O)O)noc1-c1ccc(Cl)cc1Cl]

LC-MS: Rt 2.06 min, m/z no mass detected [M+H]⁺; Method A.

### Intermediate L25 Step 1

### 3-(2,4-Dichloro-phenyl)-3-oxo-propionitrile

Acetonitrile (0.637 ml, 12.19 mmol) was added to toluene (12 ml) and cooled to −78° C. n-BuLi (1.6M, 5.33 ml, 8.53 mmol) was added and after 20 min, methyl 2,4-dichloro benzoate (500 mg, 2.439 mmol) dissolved in toluene (3 ml) was added. The reaction was allowed to warm to 0° C. The reaction was acidified with dilute HCl, the organic layer separated, dried and evaporated to dryness to give 3-(2,4-dichloro-phenyl)-3-oxo-propionitrile as a brown solid. The product was a mixture of ketone and cis and trans enoles. LC-MS: Rt 2.00 min; m/z no mass detected [MH]⁺; Method A.

### Intermediate L25 Step 2

### 4-Cyano-5-(2,4-dichloro-phenyl)-isoxazole-3-carboxylic acid ethyl ester

3-(2,4-Dichloro-phenyl)-3-oxo-propionitrile (535 mg, 2.499 mmol) was dissolved in EtOH (5 ml) and triethylamine (0.348 ml, 2.499 mmol) was added. After 20 min, ethyl chlorooximidoacetate (379 mg, 2.499 mmol) dissolved in EtOH (2 ml) was added and the reaction was stirred at room temperature over night. The reaction mixture was evaporated twice with toluene, the residue dissolved in dichloromethan and washed with HCl, NaOH and brine. The organic layers were evaporated to dryness and the crude material purified by chromatography on silica gel (hexane/ethyl acetate) to give 4-cyano-5-(2,4-dichloro-phenyl)-isoxazole-3-carboxylic acid ethyl ester as a yellow oil. LC-MS: Rt 2.37 min; m/z no mass detected [MH]⁺; Method A.

The remaining step towards intermediate L25 was performed analogously to intermediate L13 step 6.

### Intermediate L26

### 5-(2,4-Dichloro-phenyl)-4-trifluoromethyl-isoxazole-3-carboxylic acid

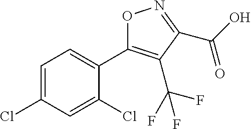 [SMILES: O=C(O)c1noc(-c2ccc(Cl)cc2Cl)c1C(F)(F)F]

LC-MS: Rt 2.25 min, m/z no mass detected [M+H]⁺; Method A.

### Intermediate L26 Step 1

### 5-(2,4-Dichloro-phenyl)-4-iodo-isoxazole-3-carboxylic acid ethyl ester

Ethyl 5-(2,4-dichlorophenyl)isoxazole-3-carboxylate (500 mg, 1.748 mmol) was dissolved in TFA (10 ml) and NIS (590 mg, 2.62 mmol) was added. The reaction was stirred at room temperature for 6 days. The reaction mixture was directly purified by chromatography on silica gel (hexane/ethyl acetate) to give 5-(2,4-dichloro-phenyl)-4-iodo-isoxazole-3-carboxylic acid ethyl ester as a pale red oil which solidified. LC-MS: Rt 2.49 min; m/z 288.0 [MH]⁺; Method A.

### Intermediate L26 Step 2

### 5-(2,4-Dichloro-phenyl)-4-trifluoromethyl-isoxazole-3-carboxylic acid ethyl ester

5-(2,4-Dichloro-phenyl)-4-iodo-isoxazole-3-carboxylic acid ethyl ester (600 mg, 1.45 mmol) and CuI (55.5 mg, 0.29 mmol) were dissolved in DMF (7 ml) and HMPA (1 ml) at room temperature. 2,2-Difluoro-2-(fluorosulfonyl) acetate (741 μL, 5.83 mmol) was added and the mixture stirred at 85° for 20 h. After cooling to room temperature, diethyl ether (100 ml) and saturated ammonium chloride solution (100 ml) were added, and the mixture stirred well. The phases were separated, the organic phase washed with brine, dried with MgSO₄ and evaporated. Chromatography on silica gel afforded 5-(2,4-dichloro-phenyl)-4-trifluoromethyl-isoxazole-3-carboxylic acid ethyl ester as a colorless oil.

LC-MS: Rt 2.59 min; m/z no mass detected [MH]⁺; Method A.

The remaining step towards intermediate L26 was performed by a similar Method to intermediate L13 step 6.

### Intermediate L27

### 5-Bromo-N-(2-cyclohexyl-1,5-dimethyl-3-oxo-2,3-dihydro-1H-pyrazol-4-yl)-4-methylisoxazole-3-carboxamide

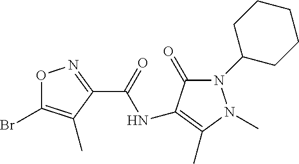 [SMILES: Cc1c(C(=O)Nc2c(C)n(C)n(C3CCCCC3)c2=O)noc1Br]

### Intermediate L27 Step 1

### Ethyl 4-methyl-5-oxo-2,5-dihydroisoxazole-3-carboxylate

Diethyl oxalpropionate (20 g, 99 mmol, 18.30 ml) and hydroxylamine hydrochloride (8.25 g, 119 mmol) were dissolved in EtOH (200 ml) and the reaction mixture was heated to 80° C. for 3 hours. The resulting mixture was partitioned between water and dichloromethane. The organic portion was separated and concentrated in vacuo to afford the title compound;

¹H NMR (400 MHz, CDCl3) δ 1.18-1.48 (m, 3H), 2.12 (s, 2H), 4.42 (q, J=7.2 Hz, 2H).

### Intermediate L27 Step 2

### Ethyl 5-bromo-4-methylisoxazole-3-carboxylate

Ethyl 4-methyl-5-oxo-2,5-dihydroisoxazole-3-carboxylate (step 1) and phosphoroxybromide were heated to 80° C. Triethylamine was added and the reaction mixture was heated at 80° C. for 3 hours. After cooling, water was added slowly and the mixture was made basic using NaOH. The mixture was extracted with dichloromethane and the combined organic extracts were dried over magnesium sulfate and concentrated under reduced pressure. Purification by flash chromatography yielded the product.

¹H NMR (400 MHz, CDCl3) δ 1.41 (t, J=7.2 Hz, 3H), 2.18 (s, 3H), 3.96-4.47 (m, 2H).

### Intermediate L27 Step 3

### 5-Bromo-4-methylisoxazole-3-carboxylic acid

To a solution of ethyl 5-bromo-4-methylisoxazole-3-carboxylate (step 2) (4 g, 17.09 mmol) in MeOH (100 ml) was added sodium hydroxide (8.55 ml, 17.09 mmol) and the resulting mixture was stirred at RT for 30 mins. The product mixture was acidified using 1M HCl (aq) and concentrated under vacuum. The resulting mixture was partitioned between water and EtOAc. The aqueous was extracted with EtOAc and combined organic extracts were dried over MgSO₄ and concentrated under vacuum to afford the title compound as a white solid;

LC-MS: Rt 0.86 mins; MS m/z 205.9 [M+H]⁺; Method 2minLowpH.

### Intermediate L27 Step 4

### 5-Bromo-N-(2-cyclohexyl-1,5-dimethyl-3-oxo-2,3-dihydro-1H-pyrazol-4-yl)-4-methylisoxazole-3-carboxamide

To a solution of 5-bromo-4-methylisoxazole-3-carboxylic acid (step 3)(3.16 g, 15.34 mmol) in EtOAc (150 ml) was added T3P® (propylphosphonic anhydride, 50% in EtOAc) (13.56 ml, 23.01 mmol) followed by triethylamine (5.35 ml, 38.4 mmol) and 2-cyclohexyl-1,5-dimethyl-3-oxo-2,3-dihydro-1H-pyrazol-4-aminium (commercial available) (3.21 g, 15.34 mmol) and the mixture was stirred at RT for 30 mins. The mixture was diluted with EtOAc and washed three times with water. The organic layer was dried over MgSO₄, filtered and concentrated under vacuum. Purification by flash column chromatography eluting with 100% TBME yielded the product as a yellow glassy solid.

LC-MS: Rt 0.92 mins; MS m/z 399.1 [M+H]+; Method 2minLowpH.

### Intermediate L28

### 5-Bromo-N-(2-(2-fluorophenyl)-1,5-dimethyl-3-oxo-2,3-dihydro-1H-pyrazol-4-yl)-4-methylisoxazole-3-carboxamide

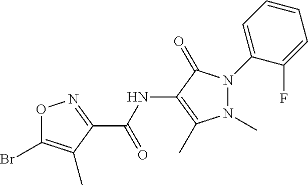 [SMILES: Cc1c(C(=O)Nc2c(C)n(C)n(-c3ccccc3F)c2=O)noc1Br]

The title compound was prepared analogously to Intermediate L27 by replacing 2-cyclohexyl-1,5-dimethyl-3-oxo-2,3-dihydro-1H-pyrazol-4-aminium (step 4) with 4-amino-2-(2-fluorophenyl)-1,5-dimethyl-1H-pyrazol-3(2H)-one (commercially available);

LCMS: Rt 0.94 mins; MS m/z 409.1 and 411.1 [M+H]+ Method 2minLowpHv01.

### Intermediate L29

### 2-(2-Chloro-4-(methoxymethyl)phenyl)-4,4,5,5-tetramethyl-1,3,2-dioxaborolane

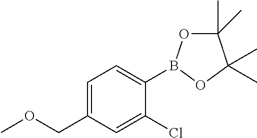

### Intermediate L29 Step 1

### 1-Bromo-2-chloro-4-(methoxymethyl)benzene

To an ice-cooled suspension of NaH (27.1 mg, 1.129 mmol) in DMF (2 ml) was added (4-bromo-3-chlorophenyl)methanol (100 mg, 0.452 mmol). The mixture was stirred for 15 mins in the ice-bath and then allowed to stir at RT for 15 mins. After this time the mixture was cooled in the ice-bath and methyliodide (0.042 ml, 0.677 mmol) was added. The mixture was allowed to warm to RT and stirred for 1.5 h. The reaction was quenched with ice water and extracted with EtOAc. The organics were washed with brine, dried over MgSO₄, filtered and concentrated under vacuum to give the title compound as a pale yellow oil;

LC-MS: Rt 1.14 mins; MS m/z no mass ion [M+H]+; Method 2minLowpH.

### Intermediate L29 Step 2

### 2-(2-Chloro-4-(methoxymethyl)phenyl)-4,4,5,5-tetramethyl-1,3,2-dioxaborolane

To a microwave vial was added 1-bromo-2-chloro-4-(methoxymethyl)benzene (88 mg, 0.374 mmol), bispinacolatodiboron (104 mg, 0.411 mmol), potassium acetate (73.3 mg, 0.747 mmol), PdCl₂(dppf)-CH₂Cl₂ adduct (15.26 mg, 0.019 mmol) and dioxane. The vial was purged with nitrogen and heated using microwave radiation at 100° C. for 1.5 hr. The resulting mixture was diluted with EtOAc and water and the layers were separated. The organics were dried over MgSO₄, filtered and concentrated under vacuum to give the title compound as a brown oil/solid. The crude product was used without further purification.

### Intermediate L75b

### 4-Cyclopropyl-5-(4-methoxy-2-(trifluoromethyl)phenyl)isoxazole-3-carboxylic acid

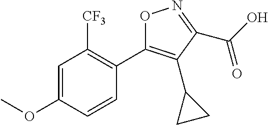 [SMILES: COc1ccc(-c2onc(C(=O)O)c2C2CC2)c(C(F)(F)F)c1]

Intermediate L75b was prepared by a similar method to that of Intermediate L23, starting from ethyl 4-iodo-5-(4-methoxy-2-(trifluoromethyl)phenyl)isoxazole-3-carboxylate (Intermediate L75a);

LC-MS: m/z 328.1 [M+H]⁺.

### Intermediate L76b

### 4-Cyclopropyl-5-(2-fluoro-4-methoxyphenyl)isoxazole-3-carboxylic acid

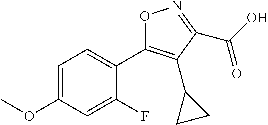 [SMILES: COc1ccc(-c2onc(C(=O)O)c2C2CC2)c(F)c1]

Intermediate L76b was prepared by a similar method to that of Intermediate L23, starting from ethyl 5-(2-fluoro-4-methoxyphenyl)-4-iodoisoxazole-3-carboxylate (Intermediate L76a);

LC-MS: m/z 278.1 [M+H]⁺.

### Intermediate L77

### 4-cyclopropyl-5-(4-methoxy-2-(trifluoromethyl)phenyl)isoxazole-3-carboxylic acid

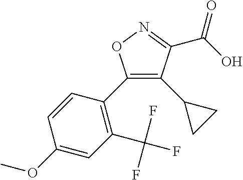 [SMILES: COc1ccc(-c2onc(C(=O)O)c2C2CC2)c(C(F)(F)F)c1]

Intermediate L77 was prepared from ethyl 4-bromo-5-(4-methoxy-2-(trifluoromethyl)phenyl)isoxazole-3-carboxylate by a similar method to that of intermediate L23 LC-MS: m/z 328.1 [M+H]⁺

We claim:

1. A compound of formula I, 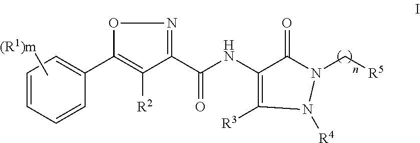
or a pharmaceutically acceptable salt thereof,
wherein:
each R¹ independently represents H, halo, C₁-C₆ alkyl, C₁-C₆ haloalkyl, C₁-C₆ alkoxy, (C₁-C₆ alkoxy)C₁-C₆ alkyl-, C₁-C₆ alkylthio, C₁-C₆ haloalkoxy, di(C₁-C₆ alkyl)amino-, C₁-C₆ alkylamino-, amino, C₃-C₇ cycloalkyl, (C₃-C₇ cycloalkyl)C₁-C₄ alkyl- or (C₃-C₇ cycloalkyl)C₁-C₄ alkoxy; or
two adjacent R¹ substituents may be taken together with the carbon atoms to which they are attached to form C₃-C₇ cycloalkyl, Het¹, or Het²;
R² represents H, halo, cyano, C₁-C₆ alkyl, C₁-C₆ haloalkyl, C₁-C₆ alkoxy, (C₁-C₆ alkoxy)C₁-C₆ alkyl-, C₁-C₆ haloalkoxy, C₃-C₇ cycloalkyl, (C₃-C₇ cycloalkyl)C₁-C₆ alkyl- or (C₃-C₇ cycloalkyl)C₁-C₆ alkoxy-;
R³ represents H, C₁-C₆ alkyl, C₁-C₆ haloalkyl, C₁-C₆ alkoxy, C₁-C₆ haloalkoxy, (C₁-C₆ alkoxy)C₁-C₆ alkyl-, (C₁-C₆ haloalkoxy)C₁-C₆ alkyl-;
R⁴ represents C₁-C₆ alkyl, which C₁-C₆ alkyl is unsubstituted or substituted by one or two halo, C₁-C₆ alkoxy or C₁-C₆ haloalkoxy groups;
R⁵ represents C₃-C₇ cycloalkyl, or phenyl, which C₃-C₇ cycloalkyl or phenyl is unsubstituted or substituted by one or two halo, C₁-C₆ alkyl, C₁-C₆ haloalkyl, C₁-C₆ alkoxy or C₁-C₆ haloalkoxy groups;
n represents an integer selected from 0 and 1;
m represents an integer selected from 0, 1 and 2;
Het¹ represents a 3 to 7 membered heterocyclic ring containing 1 or 2 heteroatoms selected from N, O and S; and
Het² represents a 5 or 6 membered heteroaryl ring containing a) 0 to 2 nitrogen atoms and 1 oxygen or sulphur atom, or b) 1 to 4 nitrogen atoms;
with the proviso that the compound of formula (I) is not one of the following compounds:
N-(2,3-dihydro-1,5-dimethyl-3-oxo-2-phenyl-2,3-dihydro-1H-pyrazol-4-yl)-5-(2-fluorophenyl)isoxazole-3-carboxamide;
N-(2,3-dihydro-1,5-dimethyl-3-oxo-2-phenyl-2,3-dihydro-1H-pyrazol-4-yl)-5-(4-fluorophenyl)isoxazole-3-carboxamide;
N-(2,3-dihydro-1,5-dimethyl-3-oxo-2-phenyl-2,3-dihydro-1H-pyrazol-4-yl)-5-(4-ethoxyphenyl)isoxazole-3-carboxamide;
N-(2,3-dihydro-1,5-dimethyl-3-oxo-2-phenyl-2,3-dihydro-1H-pyrazol-4-yl)-5-(3-chlorophenyl)isoxazole-3-carboxamide;
N-(2,3-dihydro-1,5-dimethyl-3-oxo-2-phenyl-2,3-dihydro-1H-pyrazol-4-yl)-5-(4-bromophenyl)isoxazole-3-carboxamide;
N-(2,3-dihydro-1,5-dimethyl-3-oxo-2-phenyl-2,3-dihydro-1H-pyrazol-4-yl)-5-(3,4-dimethoxyphenyl)isoxazole-3-carboxamide;
N-(2,3-dihydro-1,5-dimethyl-3-oxo-2-phenyl-2,3-dihydro-1H-pyrazol-4-yl)-5-(3,4-dimethylphenyl)isoxazole-3-carboxamide;
N-(2,3-dihydro-1,5-dimethyl-3-oxo-2-phenyl-2,3-dihydro-1H-pyrazol-4-yl)-5-(4-methylphenyl)isoxazole-3-carboxamide;
N-(2,3-dihydro-1,5-dimethyl-3-oxo-2-phenyl-2,3-dihydro-1H-pyrazol-4-yl)-5-(3,4-dichlorophenyl)isoxazole-3-carboxamide;
N-(2,3-dihydro-1,5-dimethyl-3-oxo-2-phenyl-2,3-dihydro-1H-pyrazol-4-yl)-5-(4-chlorophenyl)isoxazole-3-carboxamide;
N-(2,3-dihydro-1,5-dimethyl-3-oxo-2-phenyl-2,3-dihydro-1H-pyrazol-4-yl)-5-(4-methoxyphenyl)isoxazole-3-carboxamide;
N-(2,3-dihydro-1,5-dimethyl-3-oxo-2-phenyl-2,3-dihydro-1H-pyrazol-4-yl)-5-(3,4-difluorophenyl)isoxazole-3-carboxamide;
N-(2,3-dihydro-1,5-dimethyl-3-oxo-2-phenyl-1H-pyrazol-4-yl)-5-phenyl-isoxazole-3-carboxamide;
N-(2,3-dihydro-1,5-dimethyl-3-oxo-2-phenyl-1H-pyrazol-4-yl)-5-(2,5-dimethoxyphenyl)-isoxazole-3-carboxamide;
N-(2,3-dihydro-1,5-dimethyl-3-oxo-2-phenyl-1H-pyrazol-4-yl)-5-(3-methoxyphenyl)-isoxazole-3-carboxamide; and
N-(2,3-dihydro-1,5-dimethyl-3-oxo-2-phenyl-1H-pyrazol-4-yl)-5-(2-methoxyphenyl)-isoxazole-3-carboxamide.

2. A compound according to claim 1, wherein each R¹ independently represents halo, C₁-C₄ alkyl, C₁-C₄ haloalkyl, C₁-C₄ alkoxy, (C₁-C₄ alkoxy)C₁-C₄ alkyl-, C₁-C₄ alkylthio, C₁-C₄ haloalkoxy, di(C₁-C₄ alkyl)amino-, C₁-C₄ alkylamino-, amino, C₃-C₇ cycloalkyl, (C₃-C₇ cycloalkyl)C₁-C₄ alkyl- or (C₃-C₇ cycloalkyl)C₁-C₄ alkoxy-; or two adjacent R¹ substituents may be taken together with the carbon atoms to which they are attached to form Het¹, or Het2.

3. A compound according to claim 1 or claim 2, wherein, R² represents Br, Cl, F cyano, methyl, ethyl, trifluoromethyl, methoxy, trifluoromethoxy or cyclopropyl.

4. A compound according to any preceding claim, wherein R³ represents C₁-C₄ alkyl.

5. A compound according to any preceding claim, wherein R⁴ represents C₁-C₄ alkyl.

6. A compound according to any preceding claim, wherein R⁵ represents cyclohexyl, or phenyl, which cyclohexyl or phenyl is unsubstituted or substituted by one or two halo, C₁-C₆ alkyl, C₁-C₆ haloalkyl, C₁-C₆ alkoxy or C₁-C₆ haloalkoxy groups.

7. A compound according to any preceding claim, wherein n represents 0.

8. A pharmaceutical composition, comprising:
a therapeutically effective amount of the compound according to any one of claims 1 to 7, or a pharmaceutically acceptable salt thereof, and
one or more pharmaceutically acceptable carriers.

9. A pharmaceutical combination, comprising:
a therapeutically effective amount of the compound according to any one of claims 1 to 7, or a pharmaceutically acceptable salt thereof, and
a second active agent.

10. A method of treating pulmonary hypertension fibrosis; rheumatoid arthritis; or fracture healing; in a patient in need thereof, comprising:
administering to the subject in need thereof a therapeutically effective amount of the compound according to any one of claims 1 to 7, or a pharmaceutically acceptable salt thereof.

11. A compound according to any one of claims 1 to 7, or a pharmaceutically acceptable salt thereof, for use as a medicament.

12. Use of a compound according to any one of claims 1 to 7, or a pharmaceutically acceptable salt thereof in the manufacture of a medicament for the treatment of a disorder or disease in a subject where Smurf-1 inhibitors have a beneficial effect.

13. A compound according to any one of claims 1 to 7, or a pharmaceutically acceptable salt thereof, for use in the treatment of a disorder or disease in a subject where Smurf-1 inhibitors have a beneficial effect.

14. A compound according to any one of claims 1 to 7, or a pharmaceutically acceptable salt thereof, for use in the treatment of pulmonary hypertension; fibrosis; rheumatoid arthritis; or fracture healing.

15. A method for treating a disease where Smurf-1 inhibitors have a beneficial effect, comprising:
administering an effective amount to of at least one compound according to any of claims 1 to 7, or a pharmaceutically acceptable salt thereof, to a subject in need of such treatment.

16. The method of claim 10 wherein the pulmonary hypertension is pulmonary arterial hypertension.

17. The compound of claim 14 wherein the wherein the pulmonary hypertension is pulmonary arterial hypertension.
